(19) United States
(12) Patent Application Publication
(10) Pub. No.: US 2024/0277867 A1
(43) Pub. Date: Aug. 22, 2024

(54) A PHOTORESPONSIVE DELIVERY SYSTEM BASED ON A MODIFIED PAMAM AND METHODS THEREOF

(71) Applicant: THE UNIVERSITY OF HONG KONG, Hong Kong (CN)

(72) Inventors: Weiping WANG, Hong Kong (CN); Yang ZHOU, Hong Kong (CN); Yafei LI, Hong Kong (CN)

(21) Appl. No.: 18/566,778

(22) PCT Filed: Jun. 9, 2022
(86) PCT No.: PCT/CN2022/097804

§ 371 Date: Dec. 4, 2023

Related U.S. Application Data
(60) Provisional application No. 63/209,078, filed on Jun. 10, 2021.

(51) Int. Cl. A61K 47/69; A61K 41/00; A61K 47/22

(52) U.S. Cl. CPC A61K 47/6935; A61K 41/0042; A61K 47/22

(57) ABSTRACT

Provided is a system for drug delivery comprising a modified poly (amidoamine) (“PAMAM”) comprising a photo-cleavable compound bound to one or more active agents to form a nanocomplex. Also provided is a method of treating a subject comprising administering the PAMAM nanocomplex and irradiating the nanocomplex at the target site with a light source. In particular, the modified PAMAM is a DEACM-modified PAMAM or a BODIPY-modified PAMAM. Also provided is a method for screening for PAMAM-coumarin-active agent nanocomplexes.

### CROSS REFERENCE TO RELATED PATENT APPLICATIONS

This application claims priority to U.S. Provisional Application No. 63/209,078 filed on Jun. 10, 2021, which is incorporated by reference in its entirety.

### 1. FIELD

Provided is a system for drug delivery comprising a modified poly(amidoamine)(“PAMAM”) comprising a photocleavable compound bound to one or more active agents to form a nanocomplex. Also provided is a method of treating a subject comprising administering the PAMAM nanocomplex and irradiating the nanocomplex at the target site with a light source. In particular, the modified PAMAM is a DEACM-modified PAMAM or a BODIPY-modified PAMAM. Also provided is a method for screening for PAMAM-coumarin-active agent nanocomplexes.

### 2. BACKGROUND

Protein, an important class of biomacromolecules, performs complex and indispensable functions for organism growth and maintenance [1]. Over the past decades, there are emerging interests in using active proteins for various biological applications, such as cancer therapy [2], gene therapy [3], and vaccination [4]. Extracellular delivery of protein therapeutics, such as insulin and nimotuzumab, has made great progress in clinic [5]. However, development on cytosolic protein delivery focusing on intracellular targets is still challenging, mainly due to large molecular weights, low cellular affinity, and poor endosome-escape ability of proteins [6]. Moreover, potential risks of off-targeting delivery still exist in many current protein delivery systems [7]. Therefore, developing strategies for precise and efficient intracellular protein delivery is highly desired in the pharmaceutical industry [8].

Nano delivery systems are a commonly used platform for protein delivery. They could load proteins through either chemical conjugation or physical encapsulation [9]. Chemical conjugation, such as covalent conjugation with polyethylene glycol [10] and polyethyleneimine (PEI) [11], could achieve tight protein binding with carrying materials, but the synthesis might affect protein functionalities and increase production costs [12]. The encapsulation systems, such as micelles [13-16], lipid nanoparticles [17, 18], and silica nanoparticles [19, 20], could carry entrapped proteins to designated sites. However, the low encapsulation efficiency, especially for proteins with large molecular weights, has limited their overall performance [21]. To address these issues, co-assembled protein delivery systems through physical adsorption were developed [22, 23]. In these systems, polymers or ultra-small nanoparticles with protein binding moieties could co-assemble with proteins into nanoparticles with high loading content. The commonly used binding moieties usually have high affinity to proteins and include positively charged groups [24, 25], boronic acid derivates [26-28], guanidine derivates [29, 30], and fluoroalkanes [31]. However, these moieties usually have no stimuli-responsiveness to actively control protein release. For an efficient protein delivery system, the interactions between carrying materials and proteins should be tight and controllable.

To achieve controlled protein delivery, stimuli-responsive moieties are usually incorporated into the carriers. Smart materials responsive to external or internal triggers such as pH [17], sonication [32], and light irradiation [33], could provide precise control for targeted protein delivery and release, thus improving their therapeutic efficiency and reducing off-targeting side effects. For example, Nina et al. [34] reported a dendritic polymer composed of photocleavable nitrobenzyl-guanidine conjugates. Guanidine contributed to protein binding and nitrobenzyl moiety enabled photo-disassociation of this conjugate. Through this design, spatiotemporally controlled protein release and endosome escape had been achieved.

7-diethylamino-4-hydroxymethylcoumarin (DEACM) is a photocleavable coumarin derivative, widely applied in controlled drug release [35-38] and targeted drug delivery [39-41]. Its simple synthesis and high biosafety have been evaluated in many drug delivery systems [42]. More importantly, the protein interaction abilities of some coumarin derivates have been reported [43-46]. Besides protein binding, in this system, DEACM also contributed its hydrophobicity for self-assembly and photoresponsiveness for controlled protein release. Poly(amidoamine) dendrimer (PAMAM), a tree-like polymer with multiple amino groups, supported easy modification of DEACM on the surface. The PAMAM-DEACM conjugates (PD) were able to form nanocomplexes with several types of proteins. Moreover, after being coated with hyaluronic acid (HA) and bovine serum albumin (BSA) through physical adsorption, the protein nanoparticles (BH-PD/protein) exhibited high serum tolerance, providing great potential for clinical applications. Upon visible light irradiation, the protein nanoparticles were demonstrated to undergo fast disassociation and enhanced cellular uptake.

With the burgeoning development of biotechnology, protein therapeutics, which have highly specific and irreplaceable action [60, 61] are becoming revolutionary drugs for the treatments of cancer [62, 63] genetic disorder [64, 65] infection [66], etc. [67, 68]. However, most of current protein therapeutics can only reach extracellular targets due to the limited cell membrane penetration and endosome escape [69-72]. Meanwhile, susceptibility to enzymatic degradation and immunogenicity hinders their further clinical applications [73]. Hence, great efforts have been made in the development of efficient cytosolic protein delivery systems, including liposomes [74-76], inorganic nanoparticles [77, 78] and polymers [79, 80, 81]. One of the most widely used delivery strategies is to form carrier-protein complexes based on to electrostatic interaction, but the interactions are instable in plasma due to the strong ionic strength and serum protein binding competition [82]. Therefore, cytosolic protein delivery systems with high serum stability are of great necessity for the clinical translation of protein therapeutics [83].

Stimuli-responsive carriers are of great interest for drug delivery because of their precise targeting capacity [84]. Stimuli-responsive drug delivery and release can reduce non-specific drug distribution and off-target delivery [85]. Over the past decades, tremendous efforts have been made to develop stimuli-responsive cytosolic protein delivery carriers. Most of the strategies focus on internal stimuli-triggered drug release, such as pH-[82, 86] and redox-[87-88] responsive systems. Compared with internal stimuli, external stimuli (light, magnetic field, and ultrasound) exhibit better controllability in targeting delivery [89]. Among them, light is a promising stimulus that can be used for protein delivery as it has excellent spatiotemporal controllability [90, 91]. In recent years, different strategies have been explored for light-responsive cytosolic protein delivery, such as linking protein adhesion groups and polymers with photocleavable groups [92], directly conjugating proteins to nanoparticles with photocleavable linkers [93, 94], and using light-induced reactive oxygen species (ROS) production to release or uncage proteins [76, 95]. However, direct chemical modification and light-induced ROS may be deleterious to protein activities [96]. Additionally, several systems were designed based on UV-light which has limited tissue penetration and biosafety issues, impeding their in vivo application. Therefore, there is an urgent need to develop a long wavelength light-responsive cytosolic protein delivery platform to achieve targeting delivery without compromising the protein activity.

We successfully developed the 420 nm light-responsive polymer, PAMAM-DEACM conjugate, for photo-enhanced cytosolic protein delivery [97]. This system was proven to be applicable in intracellular delivery of negatively charged glucose oxidase (GO_{X}) and bovine serum albumin (BSA). To meet the growing needs, the light wavelength should be further elongated to extend the application of such system in delivery of a larger variety of cargo proteins for more diseases. Moreover, it is important to deeply explore and clearly elucidate the mechanism of photo-enhanced protein internalization to provide guidance for light-triggered protein delivery system development.

### 3. SUMMARY

Development of cytosolic protein delivery platforms brings new possibilities for various incurable diseases. Despite strategies based on polymer/protein self-assembly that have been recently reported, versatile self-assembly-based photo-controlled platforms for protein delivery has not yet been demonstrated. Numerous recently developed therapies have highlighted the advantages of using proteins as therapeutics. However, in many protein delivery systems, complicated carrier designs, low loading content, and off-targeting phenomenon have limited their clinical applications. Here we present a simple nano protein delivery system with high delivery efficiency and precisely photocontrolled protein release. The carrier was simply prepared by modifying the photocleavable molecule, DEACM, onto the surface of cationic dendrimer, PAMAM, in which DEACM simultaneously contributed to protein binding, self-assembly of the system, and photocontrolled protein release. This is the first time to report the protein binding ability of DEACM, and this simple system does not require complex organic synthesis nor protein modification. The high delivery efficiency and photoenhanced cellular uptake have been proved in functional proteins, making it widely applicable for various therapies.

This summary describes several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of features.

The presently disclosed subject matter provides a compound comprising a photocleavable compound bound to one or more active agents to form a nanocomplex. In one embodiment, the photocleavable compound is DEACM. In some embodiments, at least one bond between the photocleavable compound and the one or more active agents is broken and/or cleaved when the compound is exposed to light.

The following Abbreviations are used in the present disclosure:

PAMAM, poly(amidoamine); DEACM, 7-(diethylamino)-4-(hydroxymethyl)-coumarin; BFP, BODIPY-F2-modified PAMAM; HHcB protein NPs, Human serum albumin and HA coated BMP protein nanoparticles; BMP, BODIPY-Me2-modified PAMAM; BEP, BODIPY-Et2-modified PAMAM; BPP, BODIPY-Pr2-modified PAMAM; FRET, fluorescence resonance energy transfer; HA, hyaluronic acid; HSA, human serum albumin; Cas3, Caspase-3; HRP, horseradish peroxidase; GO_{X}, glucose oxidase; OVA, ovalbumin; TEM, transmission electron microscope; r(protein), rhodamine labeled-(protein).

Provided herein is a system for drug delivery comprising a modified poly(amidoamine)(“PAMAM”) comprising a photocleavable compound bound to one or more active agents to form a nanocomplex, wherein the photocleavable compound is 7-diethylamino-4-hydroxymethyl-coumarin (“DEACM”).

In certain embodiments, the photocleavable compound can be coumarin-based photocleavable group. The following are chemical formulas for some of the coumarin-based photocleavable groups that can be used in the compounds of the subject invention:

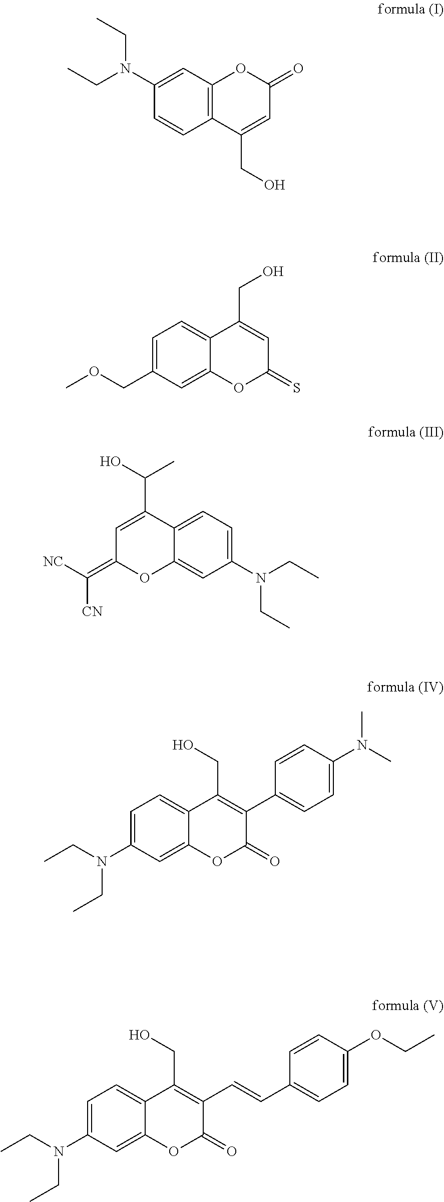

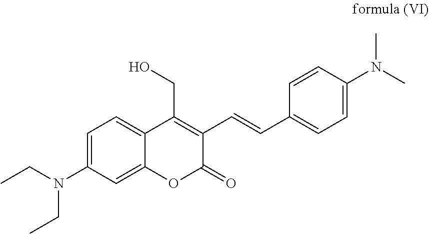 [SMILES: CCN(CC)c1ccc2c(CO)c(/C=C/c3ccc(N(C)C)cc3)c(=O)oc2c1]

In one embodiment, the system further comprising a biodegradable hyaluronic acid (“HA”) and bovine serum albumin (“BSA”) layer.

In one embodiment, the one or more active agents is an enzyme.

In one embodiment, the enzyme is glucose oxidase and caspase-3.

In one embodiment, the one or more active agents is insulin or nimotuzumab.

In one embodiment, the nanocomplex has a diameter of about 20-200 nm, or about 100-170 nm.

Provided herein is a self-assembly system for drug delivery comprising a modified PAMAM conjugated to one or more coumarin derivatives.

Provided herein is a method of delivering a drug to a subject at a target site comprising: (i) providing a PAMAM-DEACM compound that is bound to one or more active agents to form a PAMAM-DEACM active agent nanocomplex; (ii) administering the nanocomplex to the subject; and (iii) irradiating the nanocomplex at the target site with a light source to release the one or more active agents.

In one embodiment, the light source has a wavelength of 350-700 nm.

In one embodiment, the light source has a wavelength of 420-495 nm.

In one embodiment, the light source has a wavelength of 620-750 nm.

In one embodiment, the nanocomplex releases one or more active agents within 2 minutes of light irradiation at 50 mW/cm² or other time periods and irradiances.

In one embodiment, the target site is intracellular.

In one embodiment, the target site is the eye or skin.

Provided herein is a method of treating cancer in a subject in need thereof comprising: (i) administering a PAMAM-DEACM compound that is bound to glucose oxidase and/or caspase-3 to form a PAMAM-DEACM active agent nanocomplex; and (ii) irradiating the nanocomplex at the target site with a light source to release the glucose oxidase and/or caspase-3.

In one embodiment, the light source has a wavelength of 420-495 nm.

Provided herein is a method for screening coumarin derivatives for active agent delivery comprising: (i) providing coumarin derivatives conjugating with PAMAM to form PAMAM-coumarins conjugates; (ii) assembling PAMAM-coumarins and active agents to form a PAMAM-coumarin-active agent nanocomplex; (iii) measuring dispersion of the PAMAM-coumarin-active agent nanocomplex using dynamic light scattering (“DLS”); (iv) quantifying delivery efficiency of the PAMAM-coumarin-active agent nanocomplex; and (v) selecting the PAMAM-coumarin-active agent nanocomplex with the highest delivery efficiency.

In one embodiment, the coumarin derivatives have a high hydrophobicity and not photocleavable.

In one embodiment, the PAMAM-coumarin conjugates are PAMAM-coumarin-NEt2.

In one embodiment, the PAMAM-coumarin-active agent nanocomplex has more than 60 folds active agent delivery efficiency compared to a free active agent.

Provided herein is a BODIPY-modified PAMAM with excellent photo-controllability and efficiency for cytosolic active agent delivery.

In one embodiment, high serum stability was achieved by coating hyaluronic acid and human serum albumin on the surface of dendrimer/active agent nanoparticles.

In one embodiment, the modified dendrimer allowed efficient intracellular delivery of 8 cargo proteins with different charges and sizes and promoted endosome escape under green light irradiation.

In one embodiment, provided herein is a photo-triggered intracellular active agent delivery system.

In one embodiment, provided herein is a method of delivering one or more active agents comprising a BODIPY-modified PAMAM.

In one embodiment, provided herein is a method of delivering one or more active agents comprising a BODIPY derivative-based active agent delivery system.

In certain embodiments, the photocleavable compound can be BODIPY-based photocleavable group. The following are chemical formulas for certain embodiments of the BODIPY-based photocleavable groups that can be used in the compounds of the subject invention:

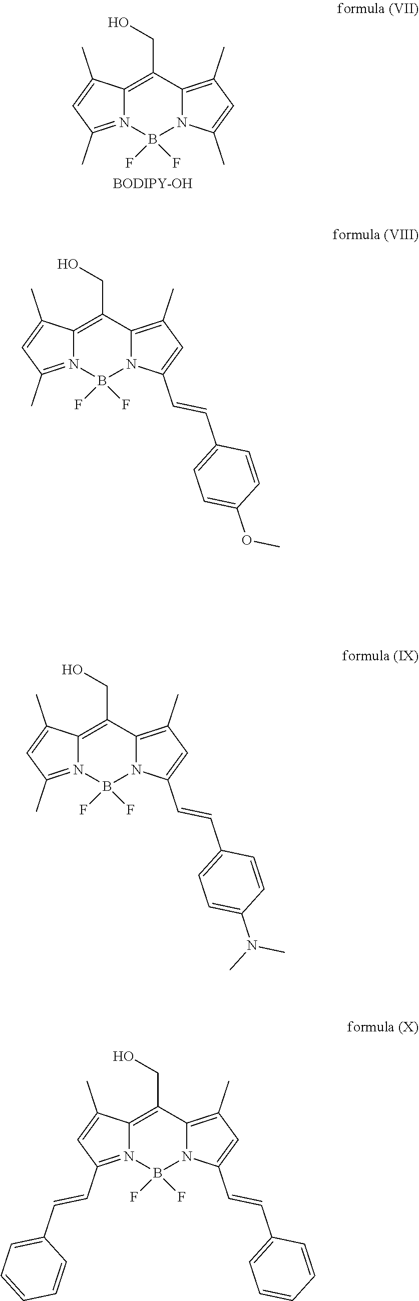

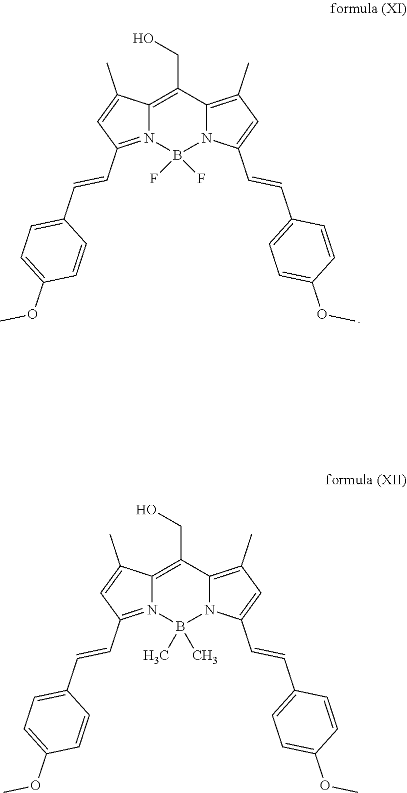

In some embodiments, the present disclosure provides that the one or more active agents is chosen from an enzyme, an organic catalyst, a ribozyme, an organometallic, a protein, a glycoprotein, a peptide, a polyamino acid, an antibody, a nucleic acid, a steroid, an antibiotic, an antiviral, an antimycotic, an anticancer agent, an anti-diabetic agent, an anti-analgesic agent, an antirejection agent, an immunosuppressant, a cytokine, a carbohydrate, an oleophobic, a lipid, an extracellular matrix, a demineralized bone matrix, a pharmaceutical, a chemotherapeutic, a virus, a virus vector, a prion and/or a combination thereof.

The presently disclosed subject matter further provides, in some embodiments, light comprising a wavelength of about 500 nm to about 1000 nm. In some embodiments, the light comprises a wavelength of about 1000 nm to about 1300 nm. In some embodiments, the light comprises a wavelength of about 500 to about 1300 nm.

In some embodiments, the compound of the present disclosure further includes a pharmaceutically acceptable carrier.

In some embodiments, provided is a method of treating a disease. The method includes the steps of administering an effective amount of a compound according to the present disclosure to a subject at an administration site, and then exposing the administration site to light.

In some embodiments, provided is a method that includes administering a compound that further includes a second active agent. In some embodiments, the second active agent is a bioactive agent. In some embodiments, the second active agent includes a second fluorophore. In some embodiments, the second active agent is chosen from an enzyme, an organic catalyst, a ribozyme, an organometallic, a protein, a glycoprotein, a peptide, a polyamino acid, an antibody, a nucleic acid, a steroid, an antibiotic, an antiviral, an antimycotic, an anticancer agent, an anti-diabetic agent, an anti-analgesic agent, an antirejection agent, an immunosuppressant, a cytokine, a carbohydrate, an oleophobic, a lipid, an extracellular matrix, a demineralized bone matrix, a pharmaceutical, a chemotherapeutic, a virus, a virus vector, a prion and/or a combination thereof.

In some embodiments, the light comprises a wavelength of about 350 to about 500 nm. In some embodiments, a wavelength of light is about 500 nm to about 1000 nm. In some embodiments, a wavelength of light is about 1000 nm to about 1300 nm. In some embodiments, a wavelength of light is about 600 nm to about 900 nm.

In some embodiments, the administration site is at, in or near a tumor. In some embodiments, non-limiting examples of the disease to be treated includes at least one of rheumatoid arthritis, cancer, and diabetes.

In some embodiments, the method includes administering a compound via at least one of oral administration, transdermal administration, inhalation, nasal administration, topical administration, intravaginal administration, ophthalmic administration, intramural administration, intracerebral administration, rectal administration, parenteral administration, intravenous administration, intra-arterial administration, intramuscular administration, subcutaneous administration, and any combination thereof.

In some embodiments of the present disclosure, a method of treating a disease is provided. The method comprises administering the drug delivery system described herein, to a subject at an administration site; and then exposing the subject and/or the administration site to light, wherein the light has a particular wavelength as described herein.

In some embodiments, the present disclosure provides a method of treating a disease, wherein the method comprises administering to a subject a compound comprising a first active agent that is appended to a photocleavable compound, wherein at least one bond between the first active agent and the photocleavable compound is broken when the compound is exposed to light having a first wavelength and further wherein at least one additional bond between the first active agent and the photocleavable compound is broken when the compound is exposed to light having a second wavelength. In some embodiments of the disclosed method(s), the compound also comprises a second active agent chosen from a fluorophore, an enzyme, an organic catalyst, a ribozyme, an organometallic, a protein, a glycoprotein, a peptide, a polyamino acid, an antibody, a nucleic acid, a steroid, an antibiotic, an antiviral, an antimycotic, an anticancer agent, an anti-diabetic agent, an anti-analgesic agent, an antirejection agent, an immunosuppressant, a cytokine, a carbohydrate, an oleophobic, a lipid, an extracellular matrix or a component thereof, a demineralized bone matrix, a pharmaceutical, a chemotherapeutic, a cell, a virus, a virus vector, a prion and/or a combination thereof. In certain embodiments, the second active agent may be appended to the photocleavable compound. Further, in some embodiments, at least one bond between the second active agent and the photocleavable compound is broken when the compound is exposed to light comprising a first wavelength and/or at least one bond between the second active agent and the photocleavable compound is broken when the compound is exposed to light comprising a second wavelength. In some embodiments of the disclosed method(s), the light comprises a first and/or second wavelength between about 350 to about 500 nm, between about 500 nm and about 1300 nm; between about 500 nm and about 1000 nm; and/or between about 1000 nm and about 1300 nm.

Provided herein is a self-assembly system for drug delivery comprising a modified PAMAM conjugated to one or more photocleavable compound, wherein the photocleavable compound is BODIPY.

Provided herein is a system for drug delivery comprising a BODIPY-modified PAMAM compound (“BMP”) comprising a photocleavable compound BODIPY bound to one or more active agents to form a BODIPY-modified PAMAM active agent nanocomplex.

Provided herein is a method of delivering a drug to a subject at a target site comprising: (i) providing a BODIPY-modified PAMAM compound (BMP) that is bound to one or more active agents to form a BODIPY modified PAMAM active agent nanocomplex; (ii) administering the nanocomplex to the subject; and (iii) irradiating the nanocomplex at the target site with a light source to release the one or more active agents.

Provided herein is a method of treating cancer in a subject in need thereof comprising: (i) administering a BODIPY-modified PAMAM compound that is bound to an active agent to form a BODIPY-modified PAMAM active agent nanocomplex; and (ii) irradiating the nanocomplex at the target site with a light source to release the active agent.

### 4. BRIEF DESCRIPTION OF DRAWINGS

The patent or application file contains at least one drawing executed in color. Copies of this patent or patent application publication with color drawings will be provided by the Office upon request and payment of the necessary fee.

FIG. 1 DEACM modified dendrimer binds to protein. (A) DLS results of free BSA, PAMAM/BSA and PD/BSA nanocomplexes. (B) Protein binding efficiency of a series of PAMAM-DEACM. (C) Schematic illustration and TEM image of the self-assembled protein nanocomplex, PD/BSA. Scale bar: 200 nm. (D) Potential interactions between PAMAM-DEACM with proteins: hydrogen bond, electron-π interaction, hydrophobic interaction, and ionic interaction. Data analysis: n=3, ***p<0.001, **p<0.01.

FIG. 2 Optimization and characterization of protein nanocomplexes. (A) Schematic illustration of the HA and BSA coating and the photoinduced disassociation of the protein nanocomplex. (B) Absorption spectra of rBSA, BH-PD/BSA and BH-PD/rBSA nanocomplexes in water. (C) Zeta potential and (D) TEM images of BH-PD/BSA nanocomplexes before and after light irradiation. Scale bar: 200 nm. (E) Fluorescence spectra of rBSA, BH-PD/BSA, BH-PD/rBSA, and BH-PD/rBSA after light irradiation in water. (F) Fluorescence intensity at 474 nm of BH-PD/rBSA in water or PBS containing 5% (w/w) BSA before and after irradiation. λ_{eX}=405 nm. Irradiation condition: 420 nm, 50 mW/cm², 2 min. Data analysis: n=3, ***p<0.001

FIG. 3 Cytosolic delivery of BSA. (A) Cellular uptake of free rBSA and rBSA nanocomplexes. Fluorescence intensity of free rBSA-treated groups were set as the control groups. Endocytosis pathway analysis of BH-PD/rBSA nanocomplexes (B) without and (C) with light treatment (EIPA, macropinocytosis inhibitor; Genistein, caveolae-mediated endocytosis inhibitor; CPZ, clathrin-mediated endocytosis inhibitor; M-3-CD, lipid-raft-mediated endocytosis inhibitor). (D) Cellular uptake of BH-PD/rBSA nanocomplexes with or without free HA treatment. (E) Confocal microscope and (F) flow cytometry analysis of cellular uptake of free rBSA, rBSA with Pierce™ Protein Transfection Reagent Kit, rBSA nanocomplexes, and rBSA nanocomplexes with light irradiation in the medium with or without serum. Scale bar: 20 m. Irradiation condition: 420 nm, 50 mW/cm², 2 min. Data analysis: n=3, **p<0.01, ***p<0.001.

FIG. 4 Enzymatic activity analysis of GO_{X} nanocomplexes. (A) Diagram of TMB protocol. GO_{X} oxidizes glucose to produce H₂O₂, and HRP catalyzed H₂O₂ to oxidize colorless TMB into blue product (TMBNH). The absorbance at 370 nm is used to determine the enzyme activity. (B) GO_{X} enzyme activity determined from the supernatant of free GO_{X}, PD/GO_{X}, PD/GO_{X} with heparin in water. (C) GO_{X} enzyme activity of GO_{X} and BH-PD/GO_{X} in PBS with 5% (w/w) BSA at 37° C. after 16 h. (D) GO_{X} enzyme activity determined from the supernatant of GO_{X}, BH-PD/GO_{X} before and after irradiation (420 nm, 50 mW/cm², 2 min) in PBS containing 5% (w/w) BSA.

FIG. 5 Cytosolic delivery of cytotoxic proteins. (A) Confocal images and (B) flow cytometry analysis of rGO_{X} uptake. (C) Cell viability analysis of GO_{X} treatment. (D) Cell viability analysis of caspase-3 treatment. (E) Flow cytometry analysis of cell apoptosis after caspase-3 treatment. Scale bar: 20 m. Irradiation condition: 420 nm, 50 mW/cm², 2 min. Data analysis: n=3, ***p<0.005, ****p<0.001.

FIG. 6 Screening of coumarin derivatives for protein delivery. A) Coumarin candidates for screening. B) Synthesis of coumarin-conjugated PAMAM. C) Schematic illustration of the self-assembly of proteins and PAMAM-coumarins. D) DLS results of PAMAM-coumarin/BSA nanocomplexes. E) Cellular uptake of FITC-BSA, the mixture FITC-BSA and PAMAM, and FITC-BSA nanocomplexes in serum free medium. F) Cellular uptake of FITC-BSA, the mixture FITC-BSA and PAMAM, and FITC-BSA nanocomplexes with HA) and BSA coating in complete medium. Free: free FITC-BSA only.

FIG. 7 Synthesis route and ¹H NMR characterization of PAMAM-DEACM. (A) Synthetic route of PAMAM-DEACM. ¹H NMR spectra of (B) DEACM-LG, (C) PAMAM (up), and PD0.1 (down) in d6-DMSO.

FIG. 8 UV-Vis absorption of PAMAM-DEACM. (A) Absorbance of DEACM at 376 nm in methanol. (B) Absorption spectra of PAMAM and PAMAM-DEACM in methanol. (C) Feeding and calculated molar ratio of DEACM to amino groups on PAMAM.

FIG. 9 FRET analysis for the interaction of PD0.4 with rBSA. (A) Fluorescence spectra of rBSA, PD/BSA, and PD/rBSA in water. (B) Fluorescence spectra of PD/rBSA in water, water containing 5% BSA, and PBS. λₑₓ=405 nm.

FIG. 10 Stability analysis of protein nanocomplexes. A) Fluorescence spectra of BH-PD/rBSA in water, 5% BSA and PBS. λₑₓ=405 nm. B) Size of BH-PD/BSA nanocomplexes in complete medium at 37° C. for 48 h.

FIG. 11 HPLC analysis of PD0.4 degradation under 420 nm light irradiation (50 mW/cm²) for different time periods. (A) HPLC spectra. Detection wavelength: 380 nm. (B) Quantified data from (A). n=3.

FIG. 12 Cellular uptake of rBSA, rBSA mixture, and nanocomplexes. All groups were mixed with the equal amount of HA and BSA as used in preparation of BH-PD/rBSA nanocomplexes. Light irradiation condition: 420 nm, 50 mW/cm², 2 min. Data analysis: n=3. ***p<0.001.

FIG. 13 Confocal images of A549 cells treated with the BH-PD/rBSA nanocomplexes for different time periods. Cells were incubated for 30 min, 1 h, 3 h, 6 h, and 16 h, respectively. Endosomes were stained with LysoTracker Green DND-26. Light irradiation condition: 420 nm, 50 mW/cm², 2 min.

FIG. 14 Cytotoxicity evaluation of PD0.4. Cells were treated with PD0.4 for 24 h. Light-treated groups were irradiated right after the addition of the material. Irradiation condition: 420 nm, 50 mW/cm², 2 mi (n=4).

FIG. 15. Interaction between BODIPY-modified PAMAM (BMP) and proteins. (A) Possible interaction between BMP and proteins: electron-n interaction, hydrophobic interaction, and ionic interaction. (B) Preparation of BMP protein nanoparticles with hyaluronic acid and human serum albumin coating. (C) Model proteins with different seizes and isoelectric points used in this study. HRP, horseradish peroxidase; HSA, human serum albumin; OVA, ovalbumin; GO_{X}, glucose oxidase (D) Fluorescence resonance energy transfer between rhodamine labeled HSA and BODIPY moieties. λex=480 nm (E) Stability test in DMEM complete medium at 37° C.

FIG. 16. BODIPY modified PAMAM (BMP) mediated cytosolic delivery of proteins. (A) Schematic illustration of BMP mediated cytosolic delivery of proteins. After light irradiation, the nanoparticles dissociated into positively charged pieces, promoting protein translocation. (B) rHSA delivery efficiency comparison of BMP with different grafting numbers. L: Light irradiation. (C) Photoenhanced rHSA delivery observed by confocal microscope. TEM images of the nanoparticles before (D) and after (E) light irradiation. Scale bar: 200 nm. (F) Zeta-potential measurement of uncoated NPs, coated NPs, and coated NPs after light irradiation. Endocytosis pathway analysis of HHcB60 rHSA NPs before (G) and after (H) light irradiation by flow cytometry. (I) Intracellular delivery of Caspase 3 (Cas-3) analyzed by Western blotting. Data analysis: N.S.: not significant, n=3, **p<0.01, ***p<0.001. Light: 520 nm Xe Lamp 25 mW/cm² 5 min

FIG. 17. Cytosolic delivery of fluorescent proteins and endosome escape analysis. (A) Endosome escape of HHcB60 rHSA NPs at 1 h, 2 h, 4 h, and 16 h after light irradiation, the excitation wavelength of DAPI, LysoTracker Deep Red, rHSA were 405, 647, and 561 nm, respectively. (B) Co-localization analysis of rHSA and acid endo/lysosomes using the Pearson correlation coefficient. Twenty cells were randomly selected from each group. (C) Confocal microscopy and (D) flow cytometry analysis of cellular uptake of rhodamine labeled chymotrypsin. (E) Confocal microscopy and (F) flow cytometry analysis of cellular uptake of rhodamine labeled DNase I (G) BMP mediated photo-enhanced rHSA delivery in different cell lines, 4T1, HUVEC, and MCF-7. Data analysis: n=3, ***p<0.001, Light: 520 nm Xe Lamp 25 mW/cm² 5 min

FIG. 18. Cytosolic delivery of function proteins. (A) HRP catalyzes color less substrate TMB into a blue product with 370 nm absorbance in the presence of hydrogen peroxide. (B) Cellular uptake of rhodamine labeled HRP. (C) HRP enzymatic activity in treated A549 cells by TMB assay. (C(D) GO_{X} catalyzes glucose into gluconic acid, followed with production of hydrogen peroxide. (E) Cytotoxicity of HHcB14 GO_{X} NPs and HHcB60 GO_{X} NPs before and after light irradiation in A549 cells determined by MTT assay. (F) Cytotoxicity of free RNase A, PAMAM RNase A, and HHcB60 RNase A NPs with or without irradiation in Hela cells. (G) Cytotoxicity of free chymotrypsin, PAMAM chymotrypsin, and HHcB60 chymotrypsin NPs with or without irradiation in Hela cells. (H) OVA antigen SIINFEKL cross-presentation efficiency. (I) Proliferation of OT-1 T cells after co-incubation with macrophage treated with HHcB60 HSA NPs, free OVA, and HHcB60 OVA NPs. Data analysis: n=3, ***p<0.001, Light: 520 nm Xe Lamp 25 mW/cm² 5 min.

FIG. 19. Synthesis route of BODIPY-modified PAMAM

FIG. 20. ¹H-NMR spectrum of BODIPY-OAc

FIG. 21. ¹H-NMR spectrum of BODIPY-OH

FIG. 22. ¹H-NMR spectrum of BODIPY-Me2-OH

FIG. 23. ¹H-NMR spectrum of BODIPY-F2-4NPC

FIG. 24. ¹H-NMR spectrum of BODIPY-Me2-4NPC

FIG. 25. ¹H-NMR spectrum of BODIPY-Me2-modified PAMAM

FIG. 26. Grafting number calculation

FIG. 27. Average size and PDI of the polymer/BSA complexes at different polymer-to-protein weight ratios. n=3.

FIG. 28. Fluorescence decrement of BMP14 and BMP60 (3.4 μM). λₑₓ=480 nm, Xem=530 nm

FIG. 29. Formulation of HHcb60 protein nanoparticles and DLS data (Mass ratio).

FIG. 30. Tumor penetration of rHSA in A549 tumor spheroids treated with free rHSA, PAMAM rHSA and HHcB60 rHSA NPs with or without irradiation. Light: 520 nm Xe Lamp 25 mW/cm² 5 min.

FIG. 31. UV-Vis spectra of BMP, BEP, BPP, and BFP and HHcBXP mediated photo-enhanced rHSA delivery comparison

FIG. 32. Enzymatic activity analysis of BMP GO_{X} complexes. GO_{X} oxidizes glucose to produce H₂O₂, and HRP catalyzes H₂O₂ to oxidize colorless TMB into a blue product (TMBNH). The absorbance at 370 nm is used to determine the enzyme activity. (A) GO_{X} enzyme activity determined from the supernatant of free GO_{X}, HHcB60 GO_{X} NPs, and HHcB60 GO_{X} NPs with heparin in water. (B) SDS-PAGE analysis of supernatant

FIG. 33. Biocompatibility of BMP60 before and after light irradiation in A549 cells (left) and Hela cells (right) Viability of non-treated cells was regarded as 100%.

FIG. 34. Characterization of HA-BMP/iFSP1&Ce6 NPs. (A) Fluorescent resonance energy transfer between iFSP1 and BODIPY. Excitation Wavelength: 380 nm. (B) UV-Vis spectra of Ce6, iFSP1, BMP, and BMP/iFSP1&Ce6 NPs, red box: red shift of the Ce6 characteristic peak after encapsulation. (C) Hydrodynamic size distribution of HA-BMP/iFSP1&Ce6 NPs determined by dynamic light scattering (DLS). (D) Zeta-potential of non-irradiated nanoparticles and irradiated nanoparticles. (E) Stability test of HA-BMP/iFSP1&Ce6 NPs in complete medium at 37° C. (F) Morphology of non-irradiated nanoparticles and irradiated nanoparticles observed under transmission electron microscope. Light source: 520 nm Xe Lamp, 25 mW/cm², 5 min.

FIG. 35. Photo-enhanced cellular uptake of Ce6 and iFSP1. (A) Cellular uptake of Ce6 and iFSP1 analyzed by confocal microscopy. Cells were treated with free Ce6, free iFSP1, non-irradiated HA-BMP/iFSP1&Ce6 NPs, and irradiated HA-BMP/iFSP1&Ce6 NPs, separately. Ce6 and iFSP1 were detected by the Qdot655 channel and the DAPI channel, respectively. (B) MTT assay to determine photo-enhanced drug delivery and synergism of encapsulated drugs. Cells were treated with free Ce6, free iFSP1&Ce6, HA-BMP/Ce6 NPs, HA-BMP/iFSP1&Ce6 NPs. 656 nm light (Xe Lamp, 5 mW/cm², 30 min) was utilized to sensitize photosensitizer Ce6 to produce singlet oxygen (“−”: light off, “+”: light on). 520 nm light (Xe Lamp, 25 mW/cm², 5 min) was used to cleave BODIPY from BMP. n=3, ***:p<0.001.

FIG. 36 In vivo targeting ability of HA-BMP/iFSP1&Ce6 NPs. (A) Fluorescence imaging of the biodistribution of HA-BMP/iFSP1&Ce6 NPs in 4T1-tumor-bearing BALB/c mice in vivo at an identical Ce6 concentration of 2 mg/kg, iFSP1 concentration of 0.5 mg/kg. Ce6 fluorescence was detected by IVIS® Spectrum In Vivo Imaging System at the AF647 channel. Light source: 520 nm LED lamp, 100 mW/cm², 5 min. (B) Fluorescence imaging of the major organs and tumors ex vivo examined at 24 h post-injection and light irradiation. (C) Quantified fluorescence signal of the major organs and tumors ex vivo. n=4.

FIG. 37. Characterization of HH-BMP/Ce6&rHSA NPs. (A) Hydrodynamic size distribution of HH-BMP/Ce6&rHSA NPs determined by dynamic light scattering (DLS) (B) Zeta-potential of non-irradiated nanoparticles and irradiated nanoparticles. Light source: 520 nm Xe Lamp, 25 mW/cm², 5 min.

FIG. 38. Cellular update analysis by flow cytometry. (A) Flow cytometry analysis of cellular uptake of Ce6 in the different groups, including PBS (control), the mixture of free rHSA and Ce6, the mixture of PAMAM, rHSA and Ce6, HH-BMP/Ce6&rHSA NPs in the dark, and HH-BMP/Ce6&rHSA NPs with light irradiation. (B) Quantitative analysis of the fluorescence intensity of Ce6 in 4T1 cells. (C) Flow cytometry analysis of cellular uptake of rHSA in the different groups. (D) Quantitative analysis of the fluorescence intensity of rHSA in 4T1 cells. Light source: 520 nm Xe Lamp, 25 mW/cm², 5 min. Ce6 and rHSA uptake was detected through the Qdot655 and PE channel, respectively. n=3. *** p<0.001

### 5. DETAILED DESCRIPTION

The presently disclosed subject matter includes photoresponsive protein delivery system, and in particular, certain embodiments include PAMAM that are appended to a photoresponsive ligand. In some embodiments the photoresponsive ligand is a fluorophore. In some embodiments, the photoresponsive ligand is DEACM or BODIPY.

When the photoresponsive compounds of the present disclosure are exposed to light, at least one bond between the fluorophore and PAMAM is cleaved. As used herein, the terms “photocleavable,” “photo-releasable,” “photo-activated,” “photo-responsive,” and the like are used interchangeably to describe compounds wherein one or more bonds is broken upon that compound's exposure to light.

Active agents of the present disclosure, such as proteins, include but are not limited to, enzymes, organic catalysts, ribozymes, organometallics, proteins, glycoproteins, peptides, polyamino acids, antibodies, nucleic acids, steroidal molecules, antibiotics, antivirals, antimycotics, anticancer agents, analgesic agents, antirejection agents, immunosuppressants, cytokines, carbohydrates, oleophobics, lipids, extracellular matrix and/or its individual components, demineralized bone matrix, pharmaceuticals, chemotherapeutics, viruses, virus vectors, and prions.

In some embodiments, the compounds can be tuned to be light-activated at a particular wavelength and/or over a given range of wavelengths. In some embodiments, the compounds can be tuned to be light-activated at certain wavelengths by appropriately selecting the fluorophore that is included in the compound.

In some embodiments, the compound comprises an active agent, and the compound can remain in an inert state until activated by light having a particular wavelength, thereby cleaving the active agent from the compound.

In some embodiments, the compounds can be tuned to be photo-activated by wavelengths that correspond to the wavelength of light absorbed by the fluorophore(s) appended to the compound. In some embodiments, the compounds are most rapidly activated via exposure to light having wavelengths that approximately correspond to the excitation spectrum of the appended fluorophore. In this regard, in some embodiments the compound is not photo-activated, or at least has a reduced rate of photo-activation when exposed to light having wavelengths that are shorter than those that excite the appended fluorophore.

In certain embodiments, the compound is not photo-activated, or at least has a reduced rate of photo-activation when exposed to light having wavelengths that are longer than those that excite the appended fluorophore. Furthermore, in some embodiments the compound is not photo-activated, or at least has a reduced rate of photo-activation, when exposed to light having wavelengths that are shorter than or longer than those that excite an appended fluorophore.

In this regard, the term “light” is used herein to refer to any electromagnetic radiation that can activate a compound. In some embodiments light includes ultraviolet light, visible light, near infrared light (NIR), or infrared light (IR). Compounds activated by relatively long wavelengths of light may be particularly well-suited for targeting tumors, and the like, and/or other targets that are deep in tissues, since light generally penetrates deeper into tissues as its wavelength increases. Some embodiments of compounds have the surprising and unexpected advantage of being photo-activated by light having wavelengths greater than 500 nm. Other embodiments of the compounds of the present disclosure can be photo-activated by light having wavelengths greater than 650 nm.

More specifically, as used herein, light can refer to energy having a wavelength of about 350 nm to about 1300 nm. In specific embodiments, light can refer to energy having a wavelength of about 350-400 nm, about 400-450 nm, about 450-500 nm, about 500-550 nm, about 550-600 nm, about 600-650 nm, about 650-700 nm, about 700-750 nm, about 750-800 nm, about 800-850 nm, about 850-900 nm, about 900-950 nm, about 950-1000 nm, about 1000-1050 nm, about 1050-1100 nm, about 1100-1150 nm, about 1150-1200 nm, about 1200-1250 nm, or about 1250-1300 nm.

The presently disclosed subject matter further includes pharmaceutical compositions comprising compounds as disclosed herein. Such pharmaceutical compositions may comprise at least one pharmaceutically acceptable carrier. In this regard, the term “pharmaceutically acceptable carrier” refers to sterile aqueous or non-aqueous solutions, dispersions, suspensions or emulsions, as well as sterile powders for reconstitution into sterile injectable solutions or dispersions just prior to use. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions, which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

Suitable formulations include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents.

The compositions can take such forms as suspensions, solutions or emulsions in oily or aqueous vehicles, and can contain formulation agents such as suspending, stabilizing and/or dispersing agents. Alternatively, the active ingredient can be in powder form for constitution with a suitable vehicle, e.g., sterile pyrogen-free water, before use.

The formulations can be presented in unit-dose or multi-dose containers, for example sealed ampoules and vials, and can be stored in a frozen or freeze-dried (lyophilized) condition requiring only the addition of sterile liquid carrier immediately prior to use.

For oral administration, the compositions can take the form of, for example, tablets or capsules prepared by a conventional technique with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycollate); or wetting agents (e.g., sodium lauryl sulphate). The tablets can be coated by methods known in the art.

Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-β-hydroxybenzoates or sorbic acid). The preparations can also contain buffer salts, flavoring, coloring and sweetening agents as appropriate. Preparations for oral administration can be suitably formulated to give controlled release of the active compound. For buccal administration the compositions can take the form of tablets or lozenges formulated in conventional manner.

The compounds can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt).

The compounds can also be formulated in rectal compositions (e.g., suppositories or retention enemas containing conventional suppository bases such as cocoa butter or other glycerides), creams or lotions, or transdermal patches.

The presently disclosed subject matter further includes a kit that can include a compound or pharmaceutical composition as described herein, packaged together with a device useful for administration of the compound or composition. As will be recognized by those or ordinary skill in the art, the appropriate administration-aiding device will depend on the formulation of the compound or composition that is selected and/or the desired administration site. For example, if the formulation of the compound or composition is appropriate for injection in a subject, the device could be a syringe. For another example, if the desired administration site is cell culture media, the device could be a sterile pipette.

Still further, the presently disclosed subject matter includes a method for treating diseases, such as cancer. In some embodiments, the method comprises administering a compound, including one of the compounds described herein, to an administration site of a subject in need thereof, and then exposing the administration site of the subject to light after the compound has been administered. As described above, the light in some embodiments can be a light having a wavelength of about 500 nm to about 1300 nm. In this regard, longer wavelength light can be particularly useful for targeting deep tissue.

In some methods of the present disclosure, a plurality of compounds is administered to a subject, and the administration site(s) is then exposed to light having different wavelengths in a predetermined sequence. Accordingly, in such embodiments, the administration site can be sequentially subjected to effects of different active agents in a predetermined sequence without having to administer compounds at multiple time points. Thus, a subject can be treated by different active agents merely by adjusting the wavelength of the light that the administration site is exposed to.

Still further, in some methods, the compounds, after being administered, are internalized via the endosomal pathway of a subject's cells. Subsequently, when the cells are exposed to light, the active agent can be cleaved from the compound and/or released from endosomes into the cytosol. Through this process, some embodiments are capable of not damaging cells until the cells are exposed to light having a wavelength that activates the compound.

The term “administering” refers to any method of providing a compound and/or pharmaceutical composition thereof to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition (e.g., cancer, tumors, etc.). In further various aspects, a preparation can be administered prophylactically; that is, administered for prevention of a disease or condition.

In some embodiments, a subject will be administered an effective amount of the compound. In this respect, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

Additionally, the terms “subject” or “subject in need thereof” refer to a target of administration, which optionally displays symptoms related to a particular disease, pathological condition, disorder, or the like. The subject of the herein disclosed methods can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A patient refers to a subject afflicted with a disease or disorder. The term “subject” includes human and veterinary subjects.

In some embodiments, the subject will be suffering or will have been diagnosed with one or more neoplastic or hyperproliferative diseases, disorders, pathologies, or conditions. Thus, an administration site to be exposed in a subject may be in close proximity or at the location of such a disease, condition, etc. (e.g., tumor). Examples of such diseases, conditions, and the like include, but are not limited to, neoplasms (cancers or tumors) located in the colon, abdomen, bone, breast, digestive system, esophagus, liver, pancreas, peritoneum, endocrine glands (adrenal, parathyroid, pituitary, testicles, ovaries, cervix, thymus, thyroid), eye, head and neck, nervous (central and peripheral), lymphatic system, pelvis, skin, soft tissue, spleen, thoracic areas, bladder, and urogenital system. Other cancers include follicular lymphomas, carcinomas with p53 mutations, and hormone-dependent tumors, including, but not limited to colon cancer, cardiac tumors, pancreatic cancer, melanoma, retinoblastoma, glioblastoma, lung cancer, intestinal cancer, testicular cancer, stomach cancer, neuroblastoma, myxoma, myoma, lymphoma, endothelioma, osteoblastoma, osteoclastoma, osteosarcoma, chondrosarcoma, adenoma, breast cancer, prostate cancer, Kaposi's sarcoma and ovarian cancer, or metastases thereof.

A subject may also be in need because (s)he has acquired diseases or conditions associated with abnormal and increased cell survival such as, but not limited to, progression and/or metastases of malignancies and related disorders such as leukemia (including acute leukemias (e.g., acute lymphocytic leukemia, acute myelocytic leukemia, including myeloblastic, promyelocytic, myelomonocytic, monocytic, and erythroleukemia) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma. The conditions, diseases, and the like described above, as well as those that will be apparent to those of ordinary skill in the art, are collectively referred to as “cancer” herein.

The terms “treatment” or “treating” refer to the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

With regard to the step of exposing an administration site to light, the method of exposing can be modified to meet the needs of a particular situation. Accordingly, the light can comprise sunlight, photo-optic light, and/or laser light. Further, in some embodiments, the light comprises ultraviolet light, visible light, near infrared light, or infrared light. Moreover, the light can be exposed from a laser light source, a tungsten light source, a photooptic light source, and the like. Light can also be provided at relatively specific administration site, and can be provided, for example, by the use of laser technology, fibers, endoscopes, biopsy needles, probes, tubes, and the like. Such probes, fibers, or tubes can be directly inserted, for example, into a body cavity or opening of a subject or under or through the skin of a subject, to expose the compound(s) that has been administered to the subject to light.

Light sources can also include dye lasers or diode lasers. Diode lasers may be advantageous in certain applications due to their relatively small and cost-effective design, ease of installation, automated dosimetry and calibration features, and longer operational life. Certain lasers, including diode lasers, also operate at relatively cool temperatures, thereby eliminating the need to supply additional cooling equipment. In some embodiments, the light source is battery-powered. Also, the light source can be provided with a diffuse tip or the like, such as an inflatable balloon having a scatting material.

Light can be provided to a subject at any intensity and duration that provides the required photo-activation for a particular application. In some embodiments, the methods of treatment provided in the present disclosure comprise administering relatively low doses of the compound and/or exposing an administration site to relatively low intensity light over the course of several hours or days. In some embodiments, this low-dose technique can allow for excellent tumor control while minimizing normal tissue damage.

In some embodiments of the presently disclosed subject matter, light responsive constructs function within the optical window of tissue (600-1000 nm). In some embodiments, light-responsive constructs are encoded to respond in a wavelength-specific fashion, resulting in triggering different biological actions (e.g. release of different drugs). In some embodiments, the compounds are used to treat diseases, including but not limited to rheumatoid arthritis, cancer, and diabetes.

In some embodiments, the present disclosure provides that (a) wavelengths with maximal tissue penetration (for example, 600-900 nm) are used for drug activation, (b) specific wavelengths can be encoded for different light-activatable therapeutics, thereby enabling wavelength-dependent discrimination, and (c) the photo-responsive constructs can be attached to any position on the drug/agent-of-interest, thereby eliminating the constraint that a key functionality essential for bio-molecule activity must be covalently modified with a photo-cleavable group.

The active agent binding ability of a photoresponsive group, 7-diethylamino-4-hydroxymethylcoumarin (DEACM) is used to prepare a photoresponsive active agent delivery system with high delivery efficiency and photoresponsiveness, which did not require for complex material synthesis and active agent modification. The excellent active agent binding and fast photorelease behavior of this system have been proved with functional active agents, making it widely applicable for different therapies.

The presently disclosed subject matter is further illustrated by the following specific but non-limiting examples. The examples may include compilations of data that are representative of data gathered at various times during the course of development and experimentation related to the presently disclosed subject matter.

Provided herein is an efficient photoresponsive active agent delivery system with high active agent binding ability and delivery efficiency. Under spatiotemporally controlled light irradiation, controllable active agent release and enhanced cellular uptake could be realized. Moreover, the coating of biocompatible HA and BSA achieved the high stability of the nanocomplexes in physiological solutions, which provided another advantage over many co-assembled active agent delivery systems. The active agent binding ability of the photocleavable molecule, DEACM, was reported in this study for the first time. It contributed the photoresponsivenss, active agent binding, and also the driven force for self-assembly of the system. Meanwhile, PAMAM supported the easy modification of DEACM and electronic interaction with active agents. The design simplified the active agent encapsulation procedure and provided a active agent delivery system with excellent photoresponsiveness and biocompatibility, reducing possible off-targeting side effects. High delivery efficiency, good serum tolerance, and endosome escape performance of the system implied great potential for clinical applications.

### 5.1 DEACM Contributed Protein Binding and Nanocomplex Formation

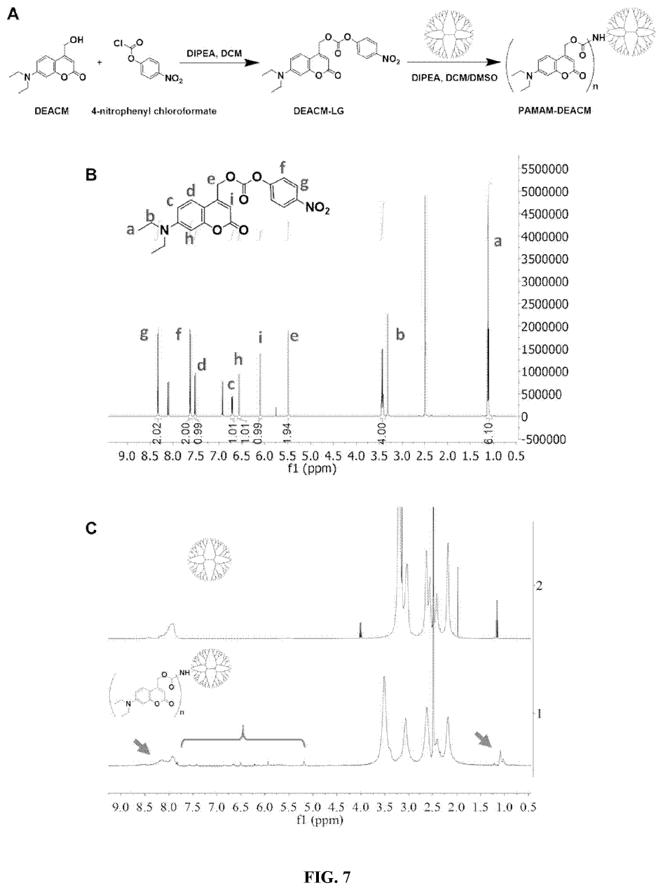

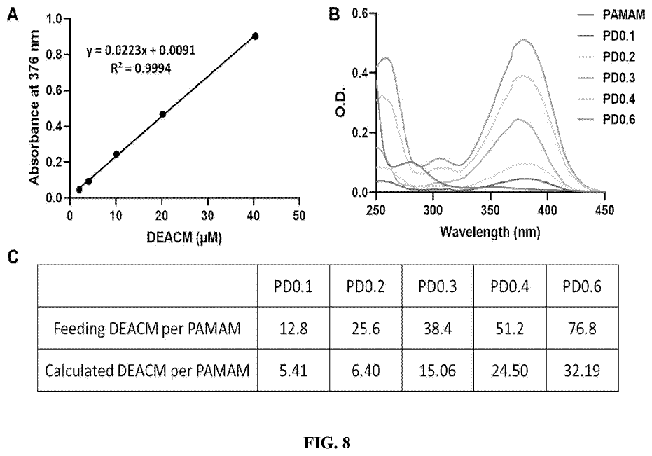

Generation five PAMAM with 128 amino groups on the surface was used in this study. The photoresponsive protein binding materials were prepared through two-step synthesis by conjugating DEACM with PAMAM (PAMAM-DEACM, FIG. 7A). According to the feeding molar ratios of DEACM to amino groups on PAMAM (1:10, 2:10, 3:10, 4:10, and 6:10), synthesized PAMAM-DEACM were noted as PD0.1, PD0.2, PD0.3, PD0.4 and PD0.6, respectively. The structures were confirmed by ¹H NMR spectra (FIG. 7B) and the grafting ratios were calculated through UV-Vis absorption spectra (FIG. 8).

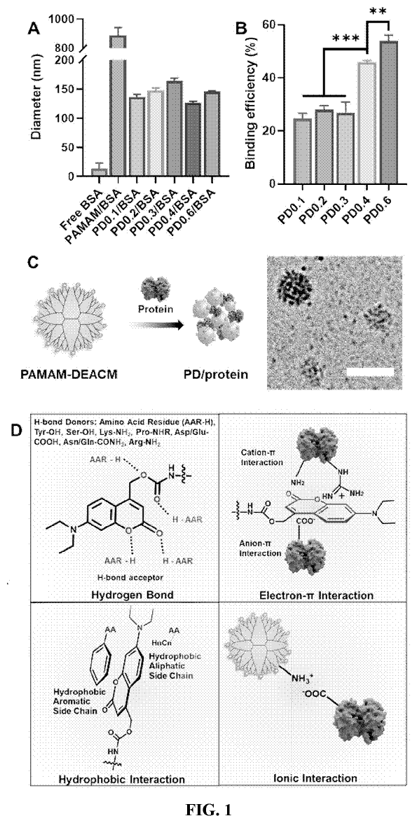

PAMAM-DEACM could form nanocomplexes in water with the model protein, bovine serum albumin (BSA), simply through the flash nanoprecipitation method [25]. The size distribution of free BSA, mixture of PAMAM with BSA, and the nanocomplexes were measured by DLS (FIG. 1A, Table Si). It should be noticed that pure PAMAM could not form uniform nanocomplexes with BSA. However, after conjugating with DEACM even though with a very low feeding ratio (PD0.1), the conjugates and BSA could form stable nanocomplexes around 140 nm in water, exhibiting the excellent protein binding ability of the DEACM group.

TABLE S1
Diameter of BSA complexes with different materials

| | PAMAM/BSA | PD0.1/BSA | PD0.2/BSA | PD0.3/BSA | PD0.4/BSA | PD0.6/BSA |
|---|---|---|---|---|---|---|
| Diameter (nm) | 887.9 | 136.0 | 147.8 | 163.8 | 126.5 | 145.5 |
| Polydispersity index | 0.849 | 0.150 | 0.206 | 0.203 | 0.179 | 0.168 |

We measured the protein binding ability with a series of PAMAM-DEACM (FIG. 1B). With more DEACM groups per PAMAM, the conjugates exhibited higher protein binding ability, which also indicated the importance of DEACM in protein loading. Considering the balance between protein binding and photoresponsiveness, PD0.4 was used for further studies. The nanocomplexes formed by PD0.4 and BSA was noted as PD/BSA, which showed a spherical and raspberry-like morphology under TEM (FIG. 1C).

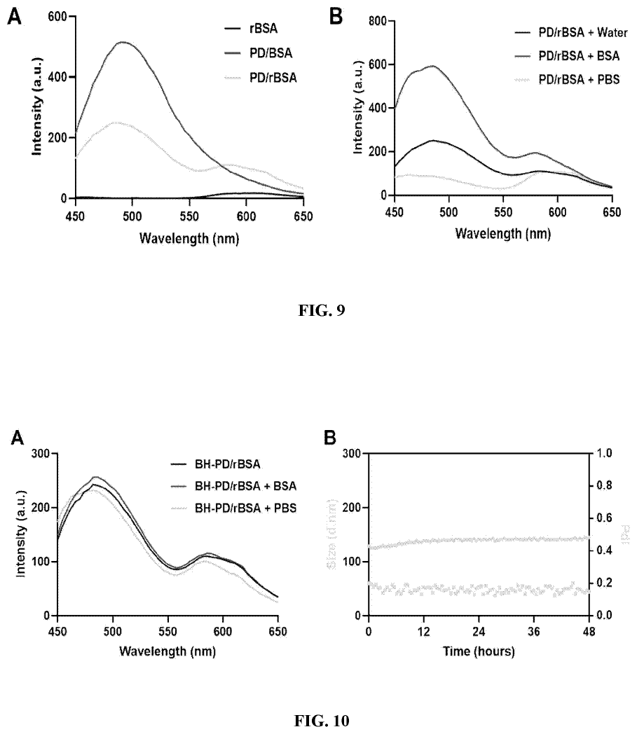

Then rhodamine labelled BSA (rBSA) was synthesized to study the interaction between PD0.4 with BSA. Rhodamine chromophore worked as the fluorescence acceptor and the fluorescent DEACM worked as the donor. The fluorescence resonance energy transfer (FRET) phenomenon between PD0.4 and rBSA demonstrated their short distance from each other and the formation of condensed nanocomplexes (PD/rBSA) in water (FIG. 9A).

Based on above results and literature studies, the possible interactions between DEACM and proteins were proposed and shown in FIG. 1D. The oxygen atoms from DEACM may contribute the binding force via hydrogen bonds [47]. The uneven electronic cloud distribution of the coumarin ring made it possible to bind with the guanidinium groups, ammonium groups, or carboxyl groups on proteins [44]. The hydrophobic structure of DEACM resulted in an organized π-π stacking or hydrophobicity guided self-assemble behavior of the protein nanocomplexes in aqueous solutions [48, 49]. Also, the remaining amino groups on PAMAM could provide additional ionic interactions with proteins [28].

### 5.2 Surface Coating Stabilized the Protein Nanocomplexes in Physiological Conditions.

Protein nanocomplexes for biomedical application would face complicate microenvironment in vivo. Therefore, preparing a stable protein delivery system capable of tolerating physiological conditions is highly demanded. Although PD0.4 could form stable protein nanocomplex with BSA in water, PD/rBSA showed dramatic fluorescence increment once added into solutions containing 5% (w/w) BSA (FIG. 9B), because the competition of free BSA with rBSA would lower the FRET effect. It also showed fast fluorescence decrement in phosphate-buffered saline (PBS) because of a homo-aggregation behavior of the nanocomplexes in buffers with high ionic concentration [50].

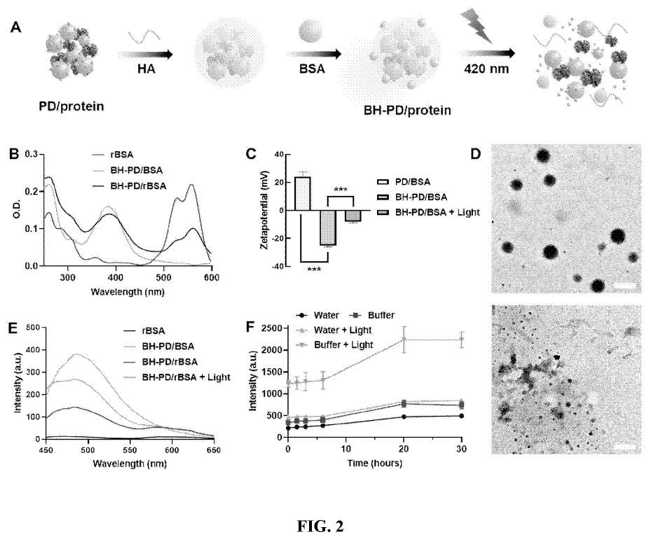

To increase the stability of nanomedicines, surface coating is generally needed [51, 52]. In this study, biodegradable hyaluronic acid (HA) and BSA protecting layers were subsequently coated on the surface to stabilize the protein nanocomplexes (BH-PD/protein, FIG. 2A). The negatively charged HA was spontaneously adsorbed onto the positively charged nanocomplexes during preparation. It could provide a negative charge for a long-term blood circulation in vivo. Meanwhile, HA endowed the nanocomplexes with a targeting ability for CD44 receptors, which are usually overexpressed on cancer cell surface, such as A549 and Hela cells [53]. Another BSA corona provided an extra steric protection for stabilization in physiological solutions [54]. The low cost, ready availability, and high biocompatibility make it wildly appliable for surface decorations [55].

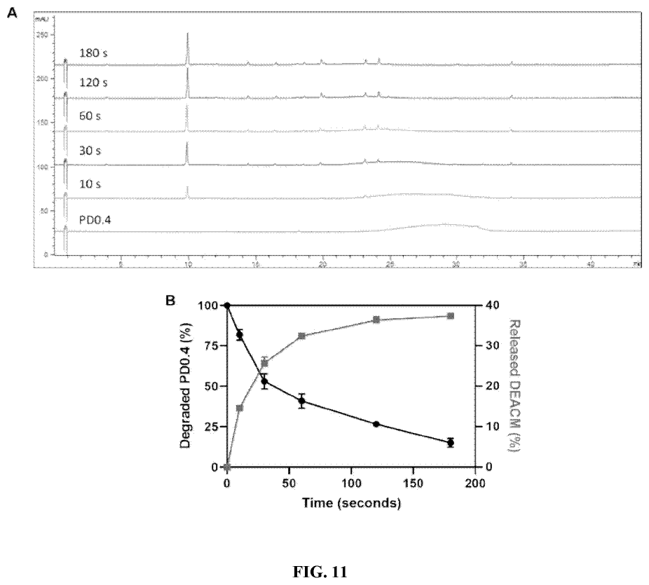

After coating and purification of BH-PD/rBSA nanocomplexes through centrifugation, absorption spectrum was recorded (FIG. 2B), which demonstrated the successful self-assembly of PD0.4 with rBSA. After coating, the zeta potential dropped from +24.0 mV to −25.2 mV. Meanwhile, the diameter of the coated nanocomplexes increased from 126.5 nm to 139.2 nm. In the darkness, BH-PD/rBSA nanocomplexes showed good tolerance to 5% (w/w) BSA solution or PBS buffer (FIG. 10A). When incubated in complete medium containing 10% (v/v) fetal bovine serum (FBS) at 37° C., the size kept almost the same for 48 h (FIG. 10B), representing a good stability of the nanocomplexes.

### 5.3 Photocleavage of DEACM Triggered Protein Release

The photoresponsiveness of the system was investigated by analyzing the photocleavage rate of PD0.4 conjugate with HPLC (FIG. 11). It showed fast degradation within 2 min under 420 nm light irradiation at 50 mW/cm², which released ˜37% DEACM with generation of some by-products [39]. The sensitive photoresponsiveness provided the foundation for photo-controlled protein release of the nanocomplexes. After light irradiation, the size of BH-PD/BSA increased to over 800 nm, and zeta potential increased to −8.0 mV (FIG. 2C), indicating the photo-induced disruption of the nanostructure. Under TEM, spherical morphology of BH-PD/BSA nanocomplexes could be observed, while disordered polymers and protein aggregates were noticed after light irradiation (FIG. 2D).

The emission spectra of BH-PD/rBSA were then measured to monitor the photo-induced DEACM cleavage and protein release. After light irradiation, due to the cleavage of DEACM and the reduced FRET effect, the fluorescence intensity of DEACM increased immediately (FIG. 2E). In PBS containing 5% (w/w) BSA, after photo-induced structure disruption, the nanocomplexes showed greater fluorescence increment than the groups in water over time (FIG. 2F). It might be because that the competition of free BSA with rBSA over the binding with DEACM accelerated the release of the rBSA. This result indicated the photo-triggered protein release of the nanocomplexes in physiological solution. Also, in the darkness over 24 h, BH-PD/rBSA only showed a slight fluorescence increment, indicating the stability of the nanocomplexes in physiological conditions.

### 5.4 Light irradiation enhanced cellular uptake of rBSA

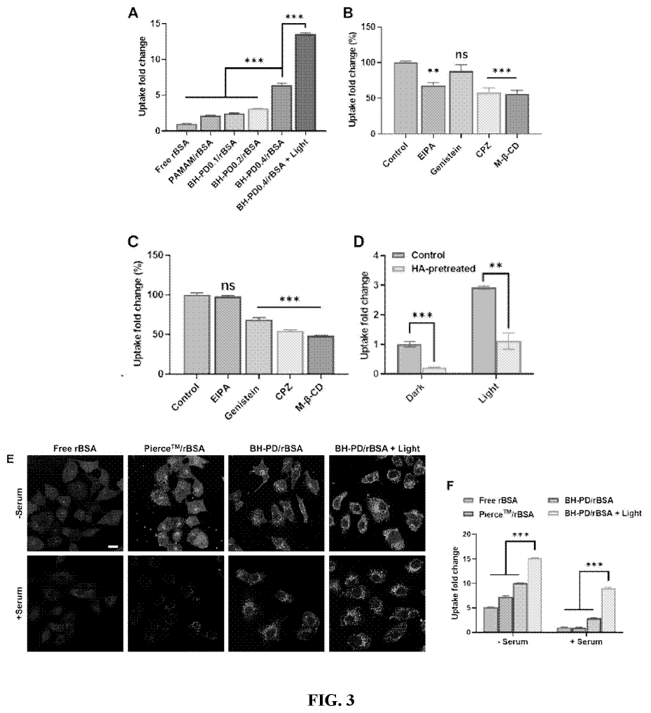

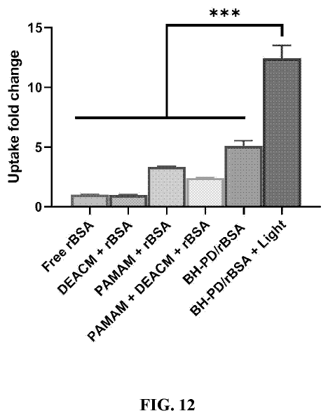

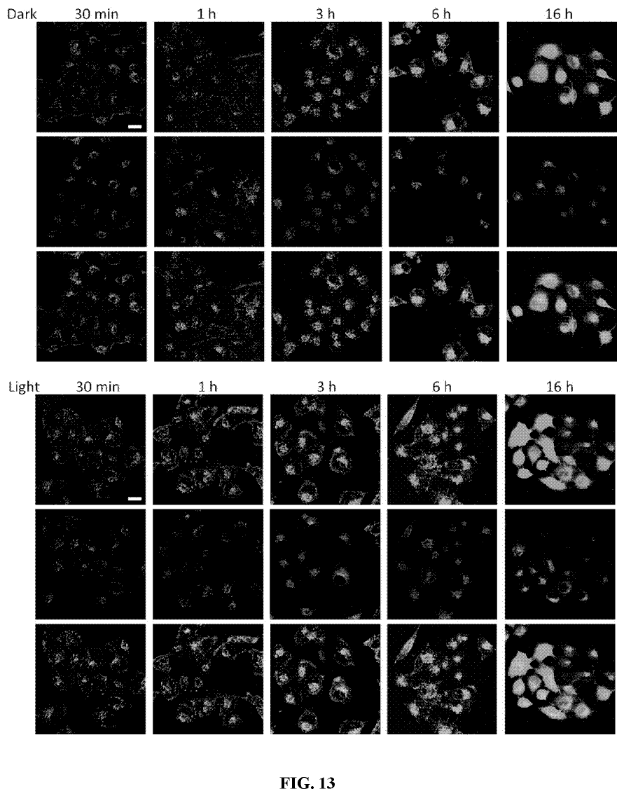

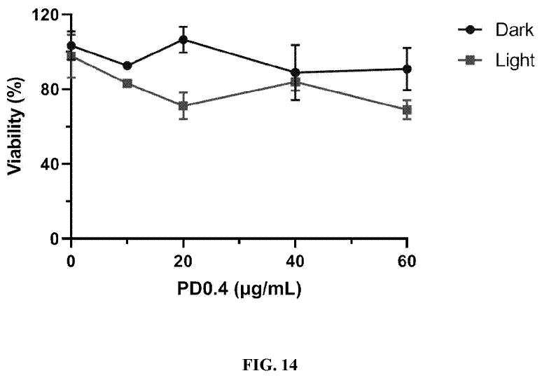

We then evaluated the intracellular protein delivery performance of the nanocomplexes using rBSA as the model protein. The red fluorescence of rhodamine was monitored to represent the protein uptake. The A549 cancer cells with overexpressed CD44 receptors were chosen in this study. As shown in FIG. 3A, the PAMAM/rBSA mixture showed slightly rBSA uptake increment than free rBSA, while BH-PD0.4/rBSA exhibited the highest rBSA delivery efficiency among all groups in the darkness. After light irradiation, the BH-PD0.4/rBSA group showed further improved cellular uptake. The increased uptake is presumably because that after the dissociation of the protein nanocomplexes, the photocleaved conjugates might still bind with protein temporarily, which had an increased surface charge to facilitate cellular uptake. To confirm the hypothesis, the cellular uptake of rBSA mixed with free DEACM and PAMAM was investigated (FIG. 12). The mixture of rBSA with DEACM or PAMAM could not sufficiently improve the protein delivery efficiency compared to BH-PD/rBSA nanocomplexes with or without light irradiation, demonstrating the unique protein delivery properties of the protein nanocomplexes.

To investigate the cellular uptake routes, cells were pretreated with four endocytosis inhibitors before the incubation with BH-PD/rBSA. In the darkness, BH-PD/rBSA uptake was more affected by macropinocytosis inhibitor (EIPA), clathrin-mediated endocytosis inhibitor (CPZ), and lipid-raft-mediated endocytosis inhibitor (M-3-CD) (FIG. 3B). After light irradiation, the uptake through macropinocytosis was less important, while caveolae-mediated endocytosis inhibitor (genistein) became more effective to inhibit complex uptake (FIG. 3C). The HA targeting performance was also evaluated by comparing complex uptake with or without pretreatment of free HA (FIG. 3D). In the darkness, due to the competition of free HA and HA coatings on the nanocomplexes, HA-pretreated group only showed ˜₂₂% cellular uptake compared with its control group. With light irradiation, HA-pretreated group showed an elevation of cellular uptake due to the exposed cationic PAMAM. However, the cellular uptake was still lower than that in the group without HA-pretreatment (˜38%). The result indicated the HA layer still contributed the cellular uptake of rBSA, which also supports the hypothesis that the pieces of dissociated nanocomplexes with HA coating might still encapsulate rBSA temporarily and enhance its intracellular delivery.

The protein uptake before and after light irradiation were then compared with a commercial protein delivery kit, Pierce™ protein transfection reagent (Thermo Fisher) through confocal microscopy (FIG. 3E) and flow cytometry (FIG. 3F). The kit showed a relative high protein delivery efficiency in serum-free medium. However, its delivery efficiency dramatically decreased in the medium containing 10% (v/v) FBS. On the contrast, BH-PD/rBSA nanocomplexes after light irradiation maintained a high protein delivery efficiency regardless of the existence of serum. The excellent serum tolerance of BH-PD/rBSA was presumably attributed to the stable BSA layer on the surface of the nanocomplexes. BH-PD/rBSA nanocomplexes also presented a time-dependent cellular uptake behavior (FIG. 13). The dislocation with acidic endosome after 6 h incubation demonstrated an efficient endosome escape ability of the protein nanocomplexes.

### 5.5 Enzyme Activity Study

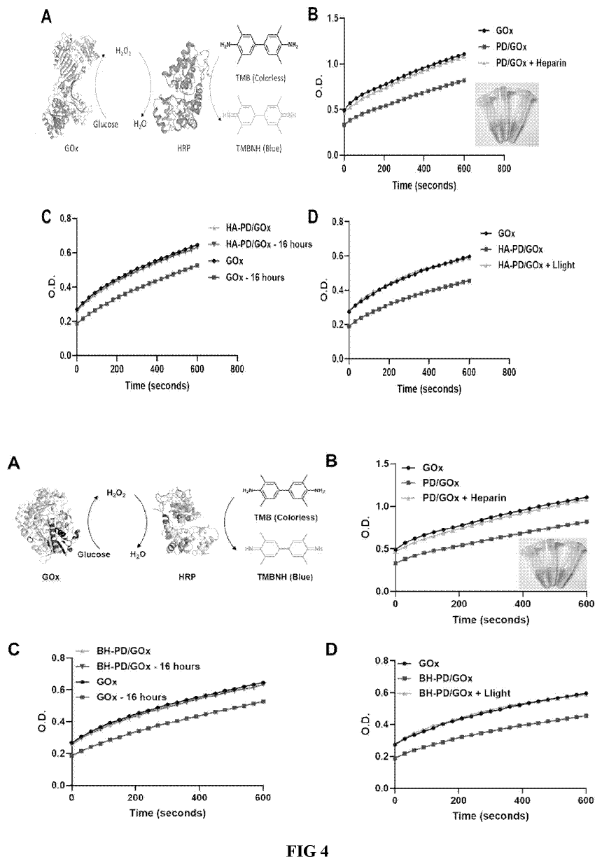

We then examined the binding ability and enzyme activity of the delivery system with a functional enzyme, glucose oxidase (GO_{X}). Through the same nanoprecipitation method, PD0.4 and GO_{X} could form stable nanocomplexes (PD/GO_{X}) around 118.6 nm in water. The PD/GO_{X} nanocomplex was collected through centrifugation and the unbound GO_{X} in the supernatant was analyzed with a standard protocol to indicate GO_{X} binding ability [56] (FIG. 4A). In principle, GO_{X} oxidizes glucose to produce H₂O₂ and horseradish peroxidase (HRP) catalyzes H₂O₂ to oxidize colorless tetramethylbenzidine (TMB) into a blue product (TMBNH). The absorbance of TMBNH at 370 nm can be used to determine the GO_{X} enzyme activity.

With the addition of heparin to compete the binding of PD0.4 against GO_{X}, the release of GO_{X} to the supernatant was observed (FIG. 4B). To prepare stable nanocomplexes in the physiological solution, the coating of HA and BSA were performed (BH-PD/GO_{X}) and the diameter of the nanocomplexes increased to 165.2 nm. The coating did not affect the enzyme activity of GO_{X}. Moreover, BH-PD/GO_{X} showed negligible enzyme activity change in PBS containing 5% (w/w) BSA over 16 h incubation (FIG. 4C), demonstrating a good stability of the system, while free GO_{X} partly lost its enzyme activity. This result demonstrated that the nanocomplex structure could slow down the protein degradation [12].

The enzymatic activity of the supernatants of BH-PD/GO_{X} nanocomplexes before and after light irradiation was measured to investigate the phototriggered release behavior (FIG. 4D). The recovered enzyme activity of the light-treated group demonstrated the release of GO_{X} after light irradiation. Thus, we concluded that nanocomplex formation and phototriggered protein release processes would not affect their enzyme activities. The nanocomplex structure could provide an excellent protection for protein delivery.

### 5.6 Cytosolic Delivery of Cytotoxic Proteins

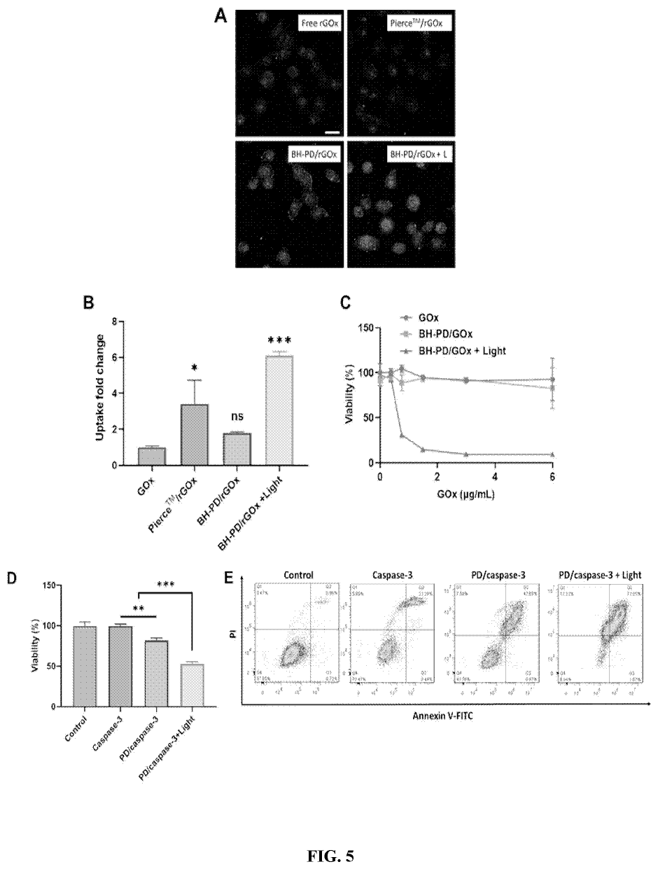

We further tested the delivery efficiency for cytotoxic proteins in vitro. GO_{X} and caspase-3 were used as protein therapeutics. GO_{X} could produce hydrogen peroxide with glucose to kill cancer cells [56]. It is a strongly anionic protein at pH 7.4, which is not preferable for cellular uptake. HA-coating endowed the nanocomplexes with binding ability to A549 cells. Moreover, light irradiation significantly enhanced cellular uptake of GO_{X}, which was confirmed by confocal microscopy (FIG. 5A) and flow cytometry (FIG. 5B). PD0.4 did not show obvious toxicity at the working concentration (<20 μg/mL, FIG. S8) against A549 cells regardless of the light irradiation, while BH-PD/GO_{X} showed high anticancer effect after the light irradiation, which may result from the fast internalization of GO_{X} complexes. Caspase-3, the activator protein for programmed cell death [57], could form ˜124.1 nm nanocomplexes with PD0.4. The light-treated PD/caspase-3 nanocomplexes showed higher cell apoptosis and death rate among all groups (FIGS. 5D and 5E), showing the successful delivery of caspase-3 into cells. These results together demonstrated that PD0.4 was a promising photocontrollable platform for cytosolic protein delivery without interfering their activity.

### 5.7 Screening of Coumarin Derivatives for Protein Delivery

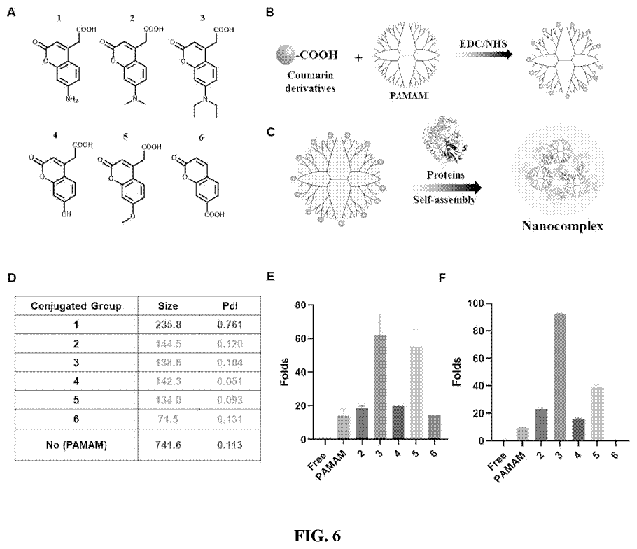

In this study, six coumarin derivatives were used to screen a coumarin molecule with the highest delivery efficiency (FIG. 6A). Coumarins were conjugated to PAMAM using 1-Ethyl-3-(3-dimethylaminopropyl)carbodiimide/N-hydroxy succinimide (EDC/NHS) reaction (FIG. 6B). PAMAM-coumarins with similar grafting ratios were used to prepare nanocomplexes with proteins (FIG. 6C). BSA was utilized as a model protein to prepare nanocomplexes with PAMAM-coumarins. The DLS results showed that coumarin-NH₂ (Molecule 1)-conjugated PAMAM cannot form well-dispersed nanocomplexes with BSA, while other derivatives with higher hydrophobicity formed nanocomplexes with BSA, indicating that hydrophobicity of coumarins is a key point for coumarin-mediated protein loading (FIG. 6D). After ruling out the coumarin-NH₂, FITC-labeled BSA (FITC-BSA) was used to quantify delivery efficiency of other PAMAM-coumarins. Coumarin-NEt₂ (Molecule 3)-modified PAMAM (PAMAM-coumarin-NEt₂) showed the highest protein delivery efficiency, whose efficiency increased to more than 60 folds compared to free protein (FIG. 6E). After coating with HA and BSA, protein nanocomplexes could be stable in complete medium. And PAMAM-coumarin-NEt₂ still showed the highest delivery efficiency among the five PAMAM-coumarins (˜90-folds) (FIG. 6F), which demonstrated the strong interaction between PAMAM-coumarin-NEt₂ and proteins.

### 5.8 BODIPY Photocleavable Compound

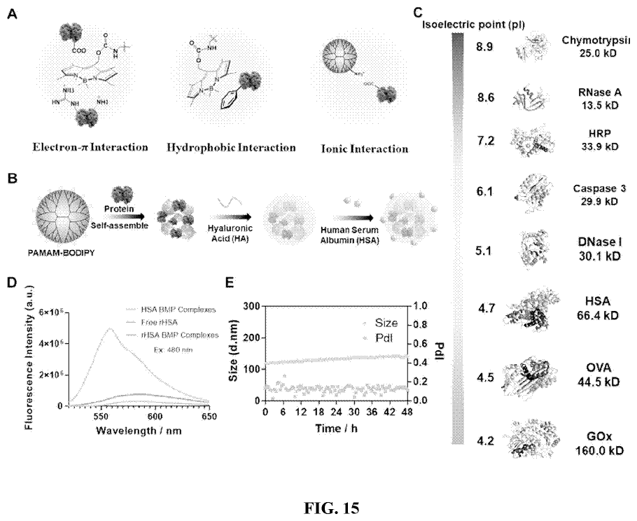

Boron-dipyrromethene (BODIPY) photoremovable protecting groups, which respond to visible to near-infrared light with high efficiency [98-100] were commonly used in photopharmacology [98-100]. Several studies have reported that BODIPY derivatives could interact with proteins [101-103]. Herein, we chose the boron-dimethyl BODIPY photoremovable protecting group [99], a BODIPY derivative with high quantum yield as the model group and conjugated the group to PAMAM to investigate BODIPY-protein interaction for nanoparticle preparation and photo-enhanced protein delivery. It is hypothesized that BODIPY-modified PAMAM could interact with proteins by electron-π, hydrophobic, and ionic interactions (FIG. 15A). In addition to promote protein binding, BODIPY groups conjugated on PAMAM could be cleaved after long wavelength light irradiation, thereby inducing charge reversal to facilitate cytosolic protein delivery. The results demonstrated that BODIPY-modified PAMAM could bind different proteins to form stable nanoparticles by self-assembly. More importantly, its high serum tolerance was achieved by coating BODIPY-modified PAMAM/proteins complexes with hyaluronic acid (HA) and human serum albumin (HSA) (FIG. 15B). Different BODIPY derivatives and grafting rates were tested to optimize the best condition for photocontrolled protein delivery. And BODIPY-modified PAMAM was demonstrated as a versatile delivery platform by intracellularly transporting various proteins with different isoelectric points and sizes (FIG. 15C).

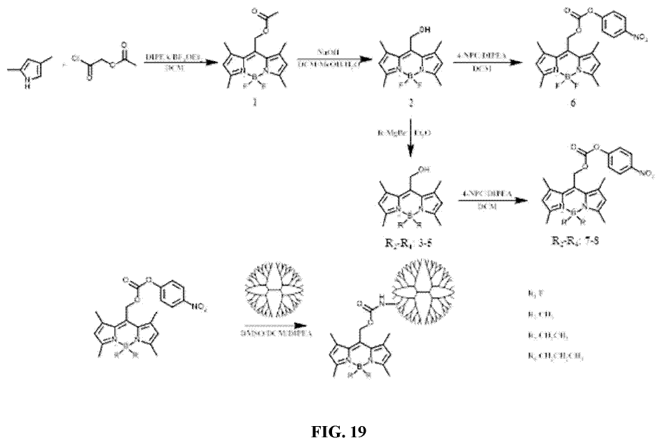

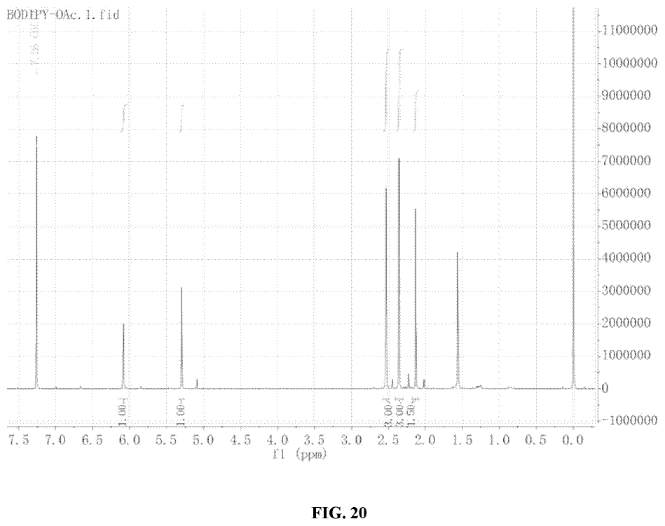

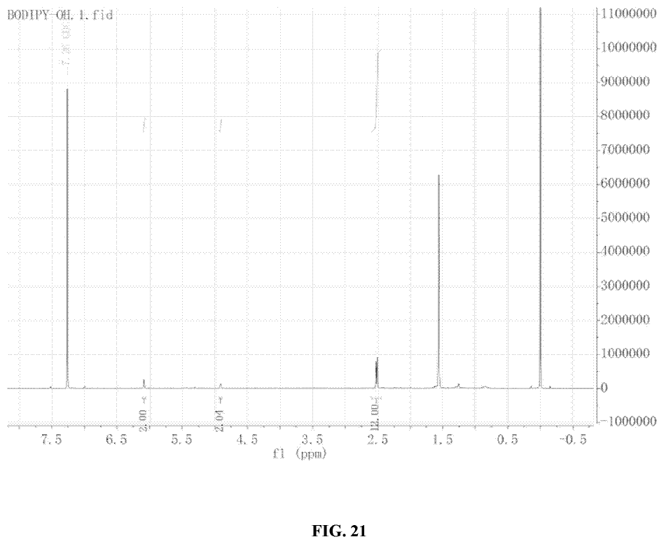

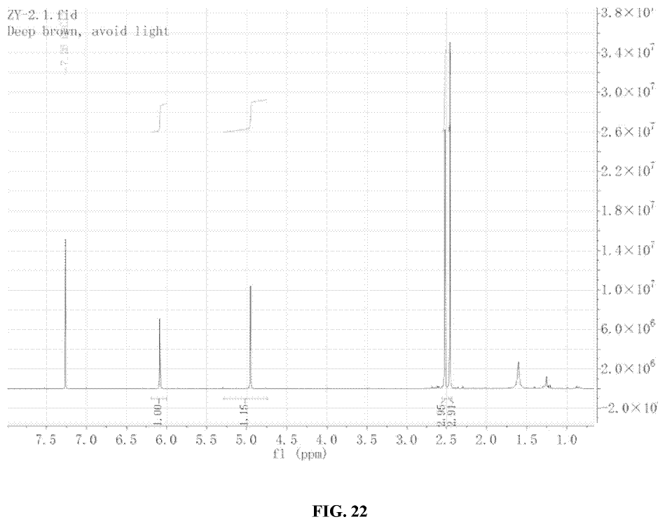

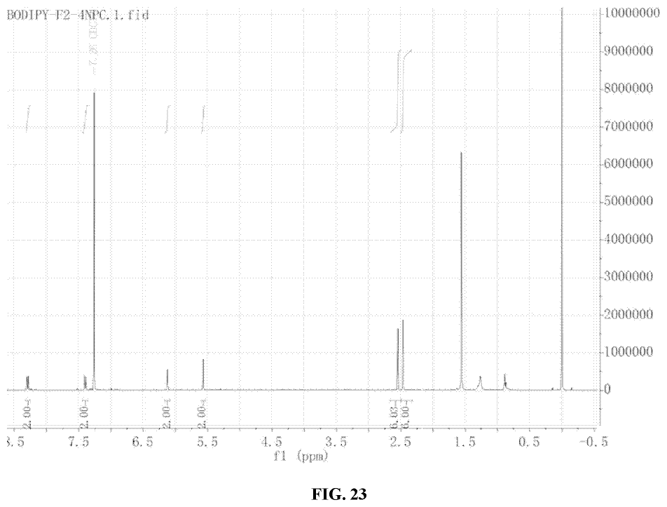

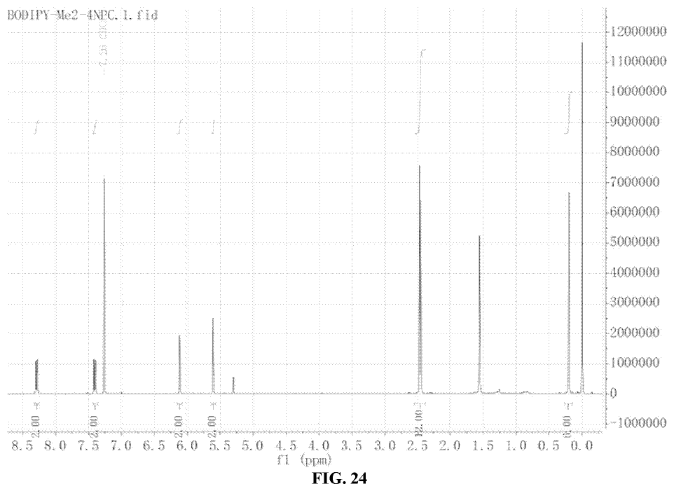

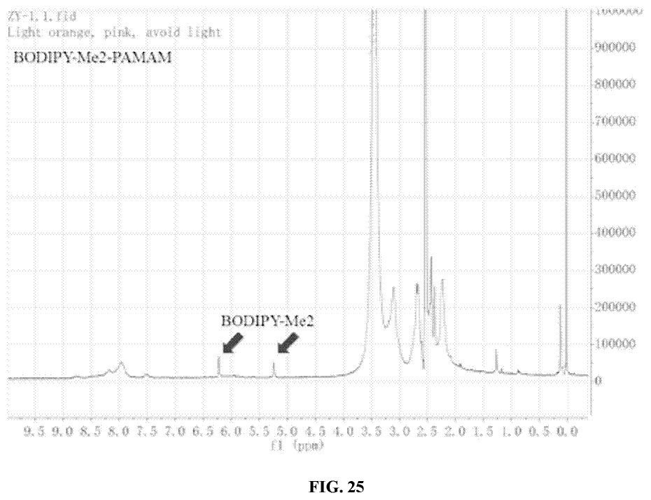

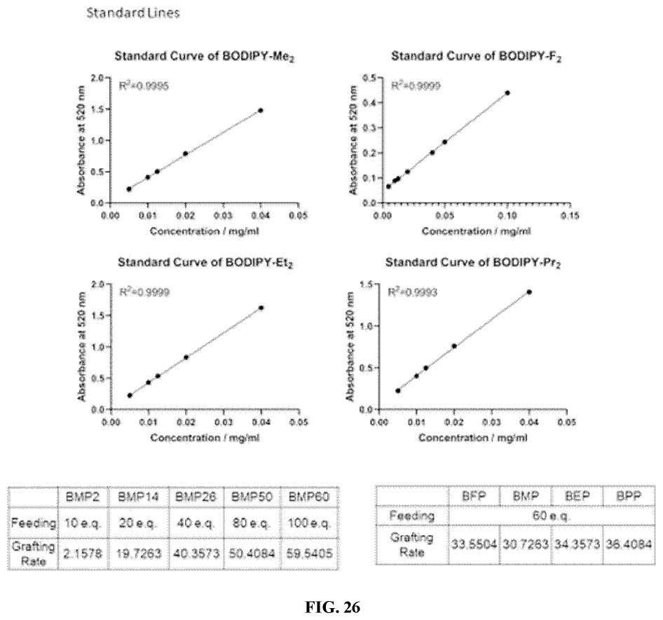

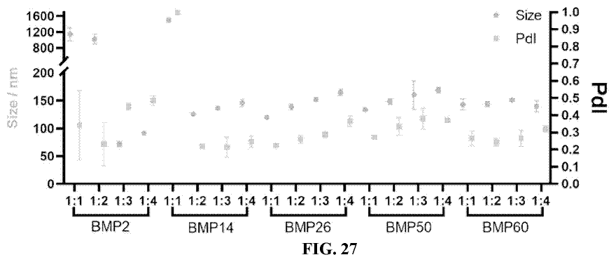

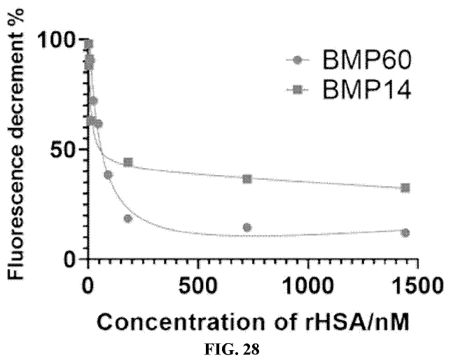

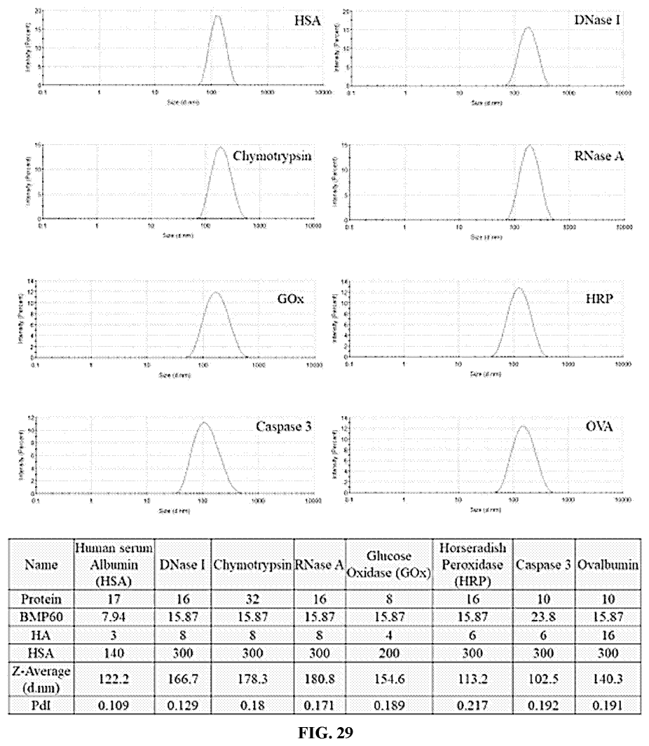

Possessing 128 terminated amine groups for protein interactions, Generation 5 (G5) polyamidoamine (PAMAM) dendrimer is a commercialized and versatile cationic polymer. The boron-dimethyl BODIPY photoremovable protecting group (BODIPY-Me₂) was activated by 4-nitrophenyl chloroformate and conjugated to PAMAM. The product was characterized by ¹H-NMR (FIG. 19-25). The BODIPY-Me₂ groups were conjugated to PAMAM at different feeding ratios to form BODIPY-Me₂-modified PAMAM (BMP). This modification enabled the BODIPY-modified polymer to respond to green light (520 nm Xenon lamp) and expose amine groups after photocleavage. The conjugated numbers of BODIPY-Me₂ on each BODIPY-modified PAMAM molecule were 2 (BMP2), 14 (BMP14), 26 (BMP26), 50 (BMP50), and 60 (BMP60), separately, quantified by UV-Vis spectra (FIG. 26). Human serum albumin (HSA) as the model cargo protein was labeled with rhodamine B isothiocyanate (RBITC) for protein delivery efficiency measurement and interaction determination. Then, we prepare complexes with BMPs and rHSA. The results showed that only BMP2 cannot form well-distributed nanoparticles with rHSA (PDI>0.3 or size >200 nm) (FIG. 27). Furthermore, interaction between BMP and RBITC-labeled HSA (rHSA) was determined by fluorescence resonance energy transfer (FRET) based on the overlap of the BODIPY emission spectrum and the rhodamine B excitation spectrum. After the formation of the rHSA-BMP60 complexes, the fluorescence intensity of BODIPY decreased significantly compared with the unlabeled HSA BMP60 complexes (FIG. 15D), indicating an obvious FRET effect and thus a strong interaction between BMP and rHSA. To further verify the interaction between BODIPY and rHSA, we selected two BMPs with different grating numbers to measure the fluorescence decrement of after feeding different amount of rHSA. As shown in FIG. 28, the fluorescence intensity of BP60 decreased much more than BMP14, suggesting that the grafting of BODIPY contributes binding force to the interaction between BMP and rHSA and higher grafting rates lead to stronger interaction. Therefore, we nominated BMP60 as the candidate carrier for protein delivery. Considering the susceptibility of BMP-protein interaction (FIG. 15A) to strong ionic strength and abundant proteins in serum, we coated the complexes with hyaluronic acid (HA) and HSA (FIG. 15B). HA was used to endow the complexes with negative charge and avoid serum proteins absorption. HSA was then applied to block all the BODIPY binding sites to prevent serum protein competition and nanoparticle aggregation in solutions with high ionic strength. Therefore, it should be noted that HSA acts as not only a model cargo protein for verification but also a stabilizer for the coating. Finally, we successfully prepared HA and HSA-coated BMP60 rHSA nanoparticles (HHcB60 rHSA NPs) with a diameter of 122.2 nm and a polydispersity index (PDI) of 0.109 by the nanoprecipitation method (FIG. 29). The nanoparticles showed high stability in complete DMEM medium at 37° C. within 48 h (FIG. 15E). The results demonstrate that BMP can interact with rHSA tightly based on BODIPY moieties modified on PAMAM. With HA and HSA protecting layers, the BMP-protein nanoparticles owned high stability against high ionic strength and binding competition of serum proteins.

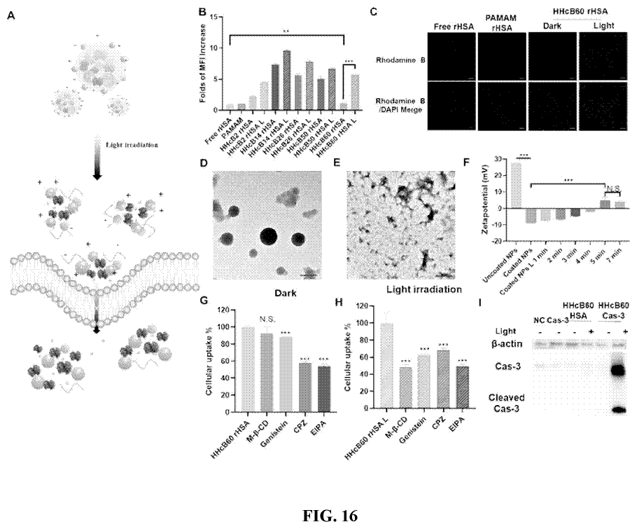

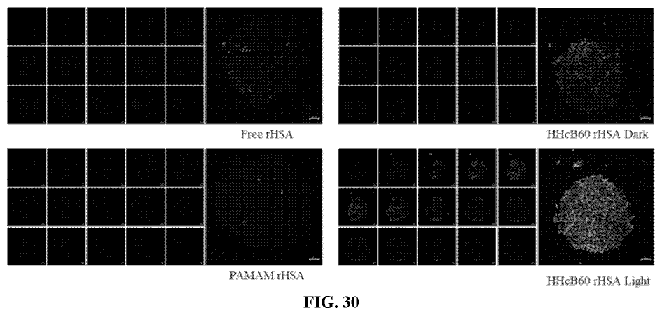

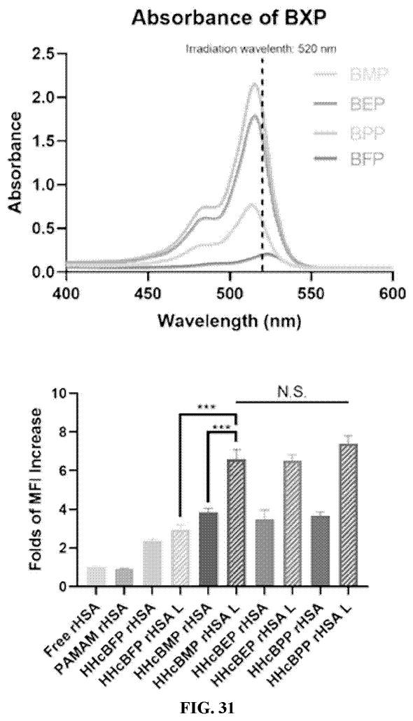

After successfully preparing stable BMP HSA NPs, we investigated whether BMPs could mediate photo-enhanced cytosolic protein delivery. Different BMPs were used to form complexes with rHSA on the preside that all the complexes were still coated with HA and HSA to determine whether the grafting numbers of BODIPY on protein delivery efficiency. The light was applied immediately after adding the complexes into cell culture media. And after 4 h incubation, cells were washed with PBS containing 20 U/mL heparin to remove unwanted proteins bound on the cell surface. The rHSA delivery efficiency to A549 cells was further quantified by flow cytometry. As shown in FIG. 16B, BMP2 performed relatively poor protein delivery efficiency even after light irradiation, which could be explained by the lower-grafting-number-caused poor protein binding ability of BMP2. Surprisingly, BMP14 exhibited the highest protein delivery efficiency among all BMP candidates, despite the relatively slight increase after light irradiation. With grafting rates increasing, BMPs showed lower protein delivery efficiency. This result suggests that PAMAM conjugated with around 14 BODIPY groups could tightly interact with rHSA. Further increasing grafting numbers, however, might lead to decreased protein delivery efficiency, which might be explained by more negative charge after coating because of less amine groups. It was also intriguing that even though BP60 nanoparticles only showed very limited protein delivery efficiency enhancement compared with naked proteins without irradiation, the intracellular protein delivery was lifted to around 6 times after light irradiation. And this significantly enhanced protein delivery efficiency might be explained by the fact that the high grafting number could induce dramatically increased surface potential change after light irradiation. Therefore, to avoid non-targeted protein delivery, we chose BMP60 for our subsequent experiments despite generally low protein delivery efficiency compared with the others. And the photo-enhanced intracellular rHSA delivery was further confirmed by confocal microscopy (FIG. 16C). In anti-cancer therapy, excellent penetration of drugs in tumor tissues could enhance efficacy. In light of this, we prepared 3D tumor spheroids to investigate the penetration ability of the nanoparticles. As FIG. 30 shown, free rHSA and PAMAM/rHSA complexes could not promote tumor penetration. And rHSA was only distributed to the edge of the spheroid in the HHcB60 rHSA NP dark group. Only after light irradiation in the HHcB60 rHSA NP group did rHSA penetrate the tumor spheroids. These results suggest BMP could not only enhance cellular uptake but also promote tumor penetration of cargo proteins in response to light irradiation. We synthesized BODIPY-F₂-modified PAMAM (BFP), BODIPY-Et₂-modified PAMAM (BEP), and BODIPY-Pr₂-modified PAMAM (BPP) in addition to BMP with close grafting rates (FIGS. 26 and 31) for comparison. All BODIPY derivatives-modified PAMAM enhanced protein delivery efficiency after the same light irradiation. However, fluorine-substituted BODIPY has poorer protein delivery efficiency than alkylated BODIPY, and meanwhile, change in the length of alkyl chain would not influence its protein delivery efficiency (FIG. 31). Based on the results shown above, we finally chose BP60 as the optimal protein delivery carrier according to the protein delivery efficiency, the photo-controllability, and the influence of boron-substitutions.

We investigated the mechanism through morphology change observation, zeta-potential measurement, and endocytosis pathway inhibition. Under transmission electron microscope (TEM), unirradiated HHcB60 HSA NPs showed spherical morphology, while the NPs turned to small pieces upon light irradiation (FIGS. 16D and 16E). We also found that the zeta-potential of the originally negatively charged HHcB60 HSA NPs with gradually turned positive charged with the irradiation periods extended (FIG. 16F). Furthermore, we used three endocytosis inhibitors, methyl-beta-cyclodextrin (M-3-CD, caveolae-dependent endocytosis), chlorpromazine (CPZ, clathrin-mediated endocytosis), and EIPA (macropinocytosis) to study endocytosis pathways of HHcB60 HSA NPs before and after light irradiation. It was shown that HHcB60 HSA NPs were mainly internalized into the cells through clathrin-mediated endocytosis and macropinocytosis (FIG. 16G). After light irradiation, we surprisingly found that cellular uptake of HHcB60 HSA NPs was significantly inhibited by M-β-CD (FIG. 16H), which obstructs caveolae-dependent endocytosis pathway that mediates 50-80 nm nanoparticle uptake [107, 108]. This result was consistent with the morphology change observed under TEM (FIGS. 16D and 16E). It is worth noting that M-β-CD inhibits caveolae-dependent endocytosis by depleting cholesterol [109]. To further demonstrate our hypothesis, we used another caveolae-dependent endocytosis inhibitor, genistein which inhibits tyrosine-kinase, different from M-3-CD. It also showed robust endocytosis inhibition of the nanoparticles after light irradiation. In summary, we showed that HHcB60 protein NPs could facilitate cytosolic protein delivery based on strong interaction between proteins and BODIPY moieties on PAMAM. In detail, the NPs could turn into dissociated and positively charged complexes after light irradiation due to photocleavage of BODIPY moieties and exposure of amine groups. Subsequently, this dissociation and charge reversal could promote endocytosis and the interaction between cell membrane and the complexes, thereby enhancing the cellular uptake of proteins.

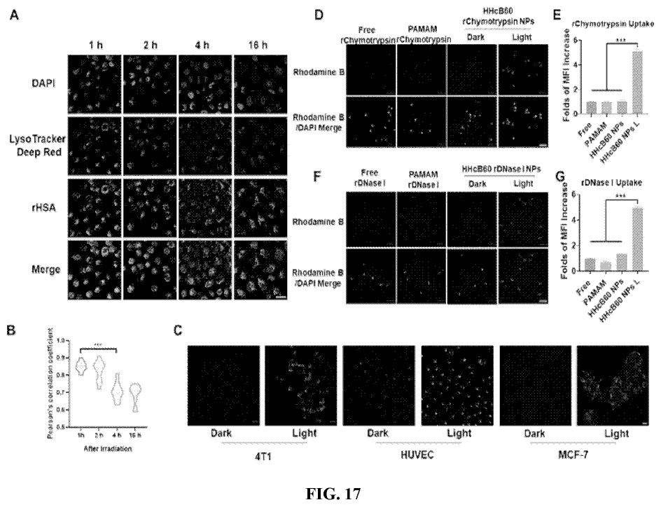

Given intracellular protein therapeutics are hindered by entrapment and subsequent degradation in acid endosome/lysosome [110], achieving effective endosome escape could ensure proteins perform their functions after intracellular delivery. We investigated whether the cargo proteins could escape from the endosome by marking the acidic compartments with LysoTracker Deep Red. After light irradiation and 2 h incubation with the nanoparticles, we observed an obvious endosome escape of rHSA suggested by the decreased yellow colocalization signal shown in FIG. 17A. And the significance of this decrease after 4 h was confirmed by the calculation of Pearson's correlation coefficient (FIG. 17B). This result indicates that the BMP protein nanoparticles could promote endosome escape after light irradiation rapidly.

As mentioned above, our strategy is based on charge reversal and dissociation-induced uptake increase, which waives the need for specific receptors. Therefore, before evaluating the protein functions, we would like to validate whether this strategy could be applied to cell lines other than A549. We then chose three cell lines from different tissues and species, 4T1 (mouse breast cancer cells), HUVEC (human umbilical vein endothelial cells), and MCF-7 (human breast cancer cells) to deliver rHSA, respectively. As shown in FIG. 17C, stronger fluorescence after light irradiation was observed in all three cell types, indicating the general applicability of our BMP platform to different cell lines.

Various proteins apart from HSA were also delivered to demonstrate the universal applicability to proteins with diverse properties. The easy preparation of HHcB60 protein nanoparticles by nanoprecipitation (FIG. 29) indicates that BMP60 does form strong interaction with proteins. Confocal microscopy and flow cytometry were used to analyze protein delivery efficiency qualitatively and quantitatively. DNase I (66.4 kD, pI 5.1, negative charge at physiological conditions) and chymotrypsin (25.0 kD, pI 8.9, positive charge at physiological conditions) were labeled with and rhodamine B isothiocyanate, respectively. As shown in FIG. 17D and FIG. 17E, the robust protein delivery efficiency facilitated by HHcB60 rChymotrypsin NPs in response to light irradiation compared with other groups was validated by both confocal microscope and flow cytometry. This result indicates that BMP60 also has strong interaction with positively charged proteins despite potential repulsion. And the similar result was found in HHcB60 rDNase I NPs (FIG. 17F and FIG. 17G). Moreover, we also delivered Caspase 3 (Cas-3, 29.9 kD, pI 6.1) and utilized western blotting to semi-quantify Cas-3 delivery efficiency. And a deep dark Cas-3 band in the HHcB60 Cas-3 NPs group was clearly observed with light irradiation (FIG. 16I). These all results suggest that BMP60 could form nanoparticles with a variety of proteins and enhance cytosolic delivery of them upon light irradiation.

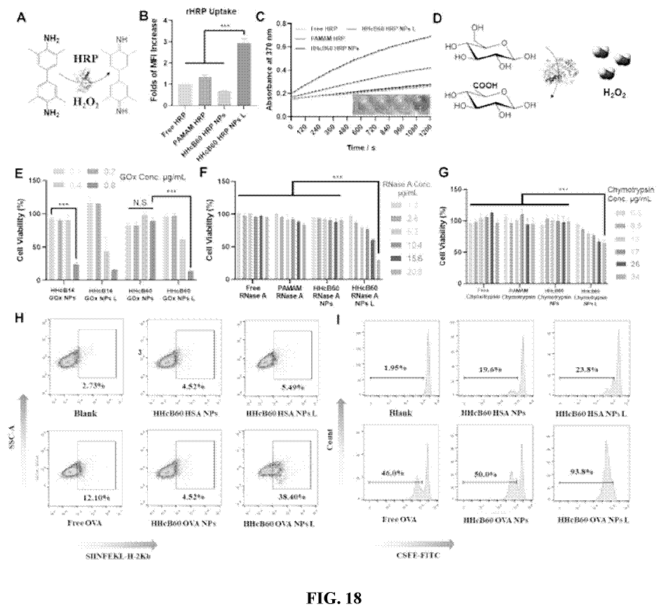

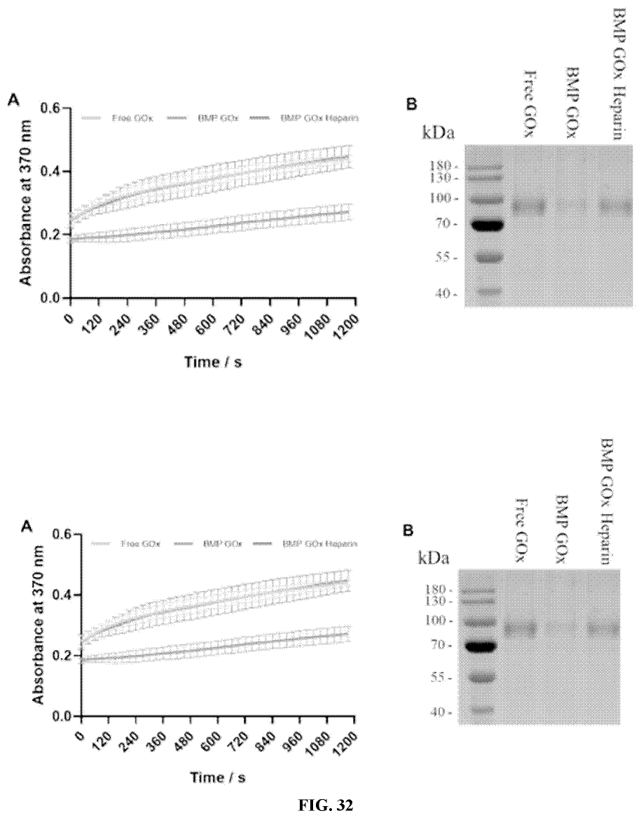

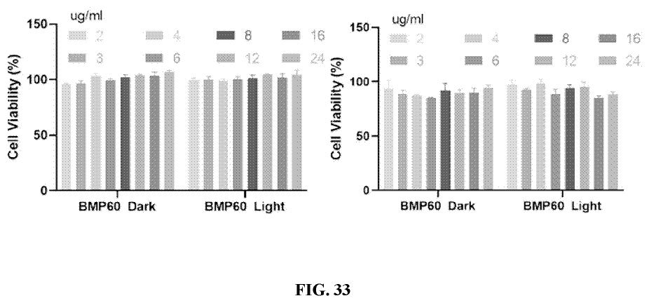

The aim of cytosolic delivery of proteins is to influence the corresponding biological process for disease precaution and treatment. Therefore, it is necessary for the protein delivery platform to maintain protein activities after cytosolic delivery. In this case, we used several bioactive proteins, horseradish peroxidase (HRP, pI 7.2, 33.9 kD), glucose oxidase (GO_{X}, pI 4.2, 160.0 kD), RNase A (pI 8.8, 13.5 kD), and chymotrypsin (pI 8.9, 25.0 kD) as model proteins to determine whether intracellular delivered proteins keep their bioactivity or catalytic functions. HRP could catalyze the oxidation of various substrates with hydrogen peroxide. Colorless 3,3′,5,5′-Tetramethylbenzidine (TMB) could be oxidated to a blue product with 370 nm absorbance in the presence of hydrogen peroxide (FIG. 18A). As shown in FIG. 4B, we found that HHcB60 rHRP NPs could enhance intracellular delivery of rHRP followed by light irradiation. As expected, the cells treated with HHcB60 HRP NPs and light illustrated the highest HRP activity determined by TMB assay (FIG. 18C). Glucose oxidase could oxidate glucose to gluconic acid. In this process, hydrogen peroxide could be produced (FIG. 18D), which can break the redox homeostasis to induce cell death. TMB colorimetric reaction and SDS-PAGE indicated the formation of HHcB60 GO_{X} complexes (FIG. 32). After incubating cells with glucose oxidase in glucose-free media, we subsequently changed the media into complete media. As FIG. 18E shown, HHcB14 GO_{X} NPs led to cell death even without light irradiation while HHcB60 GO_{X} NPs induced little death under the dark condition. Under light irradiation, both HHcB14 GO_{X} NPs and HHcB60 GO_{X} NPs triggered cell death. This result is consistent with rHSA delivery experiments (FIG. 16B), indicating that BMPs with lower grafting rates has higher protein delivery efficiency but poorer photo-controllability. RNase A is a positively charged protein under physiological conditions catalyzes the degradation of RNA chains to induce cell death. As shown in FIG. 18F, HHcB60 RNase A NPs caused significant cell death under light irradiation. Chymotrypsin is a proteolytic enzyme which promotes the cleavage of peptide bonds, thereby influencing cell metabolism and leading to cell death. Similarly, HHcB60 chymotrypsin NPs induced significant cell death after light irradiation (FIG. 18G). To further demonstrate that the decreased cell viability is mainly contributed by the enhanced protein delivery instead of material toxicity, we determined the biocompatibility of PAMAM and BMP60 with or without light irradiation. As FIG. 33 shown, we confirmed that BMP60 showed no significant cytotoxicity at the working concentration.

To further explore more possibilities of our platform for immunotherapy, we subsequently chose macrophage as the model cell. Macrophages are notoriously hard to transfect [112]. Therefore, looking for efficient strategies for intracellular delivery of proteins is important for immunotherapy. As a key type of antigen-presenting cells, macrophage could activate naïve T cells and promote subsequent immune response [113]. It is universally acknowledged that OVA₂₅₇-264 peptide (SIINFEKL) could interact with major histocompatibility complex (MHC) I molecule for cross-priming CD8⁺ T cells [114]. Therefore, we prepared HHcB60 OVA NPs nanoparticles and investigated the effect of the nanoparticles on the antigen cross-presentation. The OVA concentration was 25 ug/mL in each group. After treatment and 4 h incubation, the cell culture media were changed by fresh media. Flow cytometry assays were carried out to analyze SIINFEKL⁺ macrophage after overnight incubation. The result in FIG. 18H showed that HHcB60 OVA NPs could effectively increase the levels of SIINFEKL peptides presented on the surface of macrophages, suggesting the enhanced antigen presentation. To further validate the effect on CD8+T activation, CD8+OT-I T cells specifically recognizing ovalbumin residues 257-264 in the context of H2Kb on MIHC I were stimulated with macrophages with various treatments. Then, carboxyfluorescein diacetate succinimidyl ester (CSFE)-FITC was used to determine the proliferation of T cells in response to antigen. As FIG. 181 shown, HHcB60 OVA NPs triggered robust proliferation of OVA-induced CD8+OT-I T cells proliferation compared to resting groups after light irradiation, indicating the successful improvement of antigen presentation facilitated by our system.

We now show that BMP60 has excellent photo-controllability and high efficiency for functional protein delivery. Importantly, BMP60 could maintain protein activities and promote the antigen process after entering the cytoplasm, providing an application in different therapies in the future. Provided herein is a photo-enhanced cytosolic protein delivery platform based on BODIPY-protein interactions. This 520-nm-light-responsive BODIPY modified PAMAM could form stable nanoparticles with various proteins with different isoelectric points and sizes. Importantly, high serum tolerance of these nanoparticles promoted efficiently protein delivery and endosome escape. This is the first attempt trying to use BODIPY derivatives to deliver proteins. Based on the successful development and verification of this photo-enhanced protein delivery system, photo-triggered immunotherapy, gene editing, and other protein-based therapies will be safer and more efficient. We are now working on NIR-light-responsive BODIPY derivatives modified polymers for photocontrolled protein delivery to further enhance tissue penetration and promote clinical translation of this carrier.

### 6. EXAMPLES

### 6.1 Materials

DEACM was purchased from INDOFINE Chemical Company (New Jersey, USA). Ethylenediamine-cored and amine-terminated generation-five PAMAM dendrimer (MW: 28826 Da, 5 wt. % in methanol) and RNAase A was purchased from Sigma-Aldrich (St. Louis, MO). Before the preparation procedures, methanol was evaporated under vacuum, which gave the dendrimer as transparent gels. Rhodamine B isothiocyanate (RBITC) and human serum albumin were obtained from Macklin (China). Glucose oxidase (GO_{X}), horseradish peroxidase (HRP), and 3,3′,5,5′-Tetramethylbenzidine (TMB) were purchased from J&K Co. (Beijing, China). All other chemicals/endocytosis inhibitors/enzymes/proteins, Ethylisopropylamiloride (EIPA), Genistein, Chlorpromazine (CPZ), and Methyl-β-cyclodextrin (M-3-CD) were purchased from Dieckmann (Shenzhen, China) and used without further purification. Human Caspase 3 (Cas 3) was ordered from Sino Biological (Beijing, China). LysoTracker® Deep Red, Green DND-26, CellTrace™ CFSE Cell Proliferation Kit and Pierce™ Protein Transfection Reagent were obtained from Thermo Fisher (HK, China). All antibodies were purchases from Abcam (Hong Kong, China). All other chemicals were purchased from J&K Scientific Co. Ltd and used without further purification. Deionized water was used for all aqueous systems.

### 6.2 Synthesis and Characterization of DEACM Conjugated PAMAM (PAMAM-DEACM)

DEACM was activated with 4-nitrophenyl chloroformate (4-NPC) according to a reported procedure [39]. Generally, 170 mg DEACM (0.6878 mmol) and 1.38 g 4-NPC (6.88 mmol) were dissolved in 10 mL dry dichloromethane. N, N-Diisopropylethylamine (DIPEA, 1.2 mL, 6.85 mmol) was added slowly on an ice bath. After 15 min, the mixture was stirred at room temperature for another 6 h in the darkness. Then the reaction solution was washed with 100 mL 0.01 M hydrochloride twice. The organic layer was collected and dried over anhydrous magnesium sulfate. The crude product was purified by flash chromatography (CombiFlash® system, Teledyne ISCO, Nebraska, USA) using DCM and 2% methanol as mobile phase. Yield: 239.9 mg, 84.8%.

The activated DEACM was then conjugated with PAMAM in anhydrous DCM: DMSO (1:1, v/v) solution with a trace amount of DIPEA for 24 h in the darkness. Then the product was dialyzed (Cutoff MW: 3500 Da) against DMSO until there was no obvious DEACM fluorescence in the outer fluid. For conjugation of DEACM with different mole ratios, different amount of DEACM was used. Synthesized products were additionally dialyzed against water to remove DMSO and then freeze-dried for ¹H NMR characterization (Bruker DX 500 spectrometer at 400 Hz) and UV-Vis spectrum analysis (SpectraMax M4, Mollecular Devices).

To measure the photo-triggered release of DEACM, PD0.4 was dissolved in a mixture of methanol and water (1:1, v/v) and exposed to 420 nm light at 50 mW/cm² (Light source: Mightex LED) for different time periods. The product was then analyzed with high-performance liquid chromatography (HPLC, Agilent Technologies, 1260 Infinity II).

### 6.3 Synthesis of Fluorescent Dye-Labeled Proteins

BSA and GO_{X} were separately dissolved in phosphate-buffered saline (PBS, pH 7.4) to get 10 mg/mL protein solutions. The solutions were mixed with RBITC at a RBTIC/protein mass ratio of 1:10. The mixed solutions were stirred overnight at room temperature in the darkness. The labeled proteins were purified by dialysis (Cutoff MW: 3500 Da) against PBS and then deionized water. The purified products (noted as rBSA and rGO_{X}, respectively) were lyophilized for UV-Vis characterization and further experiments.

### 6.4 Preparation and characterization of PAMAM-DEACM/protein complexes

The complexes were prepared through flash nanoprecipitation [25]. Briefly, 20 μg PAMAM-DEACM was added into protein solutions (3-12 μg) dropwise under vigorous stirring. The formed nanocomplexes were noted as PD/protein. To further increase the stability, hyaluronic acid (HA, 16 μg) and BSA (200 μg) were subsequently added under stirring to form protective coating layers on the surface of the nanocomplexes. Then the mixture was incubated at room temperature for 30 min. The stable nanocomplexes (noted as BH-PD/protein) were diluted with water or medium for further characterizations.

The hydrodynamic diameter and zeta potential of these nanocomplexes were characterized by dynamic light scattering (DLS) using Malvern Zetasizer (Nano ZS 90, Malvern, UK). The stability test was conducted in complete DMEM medium with 10% fetal bovine serum (FBS) for 48 h at 37° C. The morphology of the nanocomplexes was observed by transmission electron microscope (TEM, Philips CM100). Forster resonance energy transfer (FRET) assay was used to determine the interaction between PD0.4 and rBSA. Rhodamine B on BSA was fluorescence acceptor to quench the fluorescence from DEACM. The fluorescence spectra were recorded at excitation and emission wavelengths of λₑₓ=405 nm and λₑₘ=450-650 nm.

The binding efficiency was measured to determine the binding ability of PAMAM-DEACM to proteins. Generally, BH-PD/rBSA nanocomplexes were prepared with PD0.1, PD0.2, PD0.3, PD0.4, and PD0.6, separately. Nanocomplexes and rBSA were centrifuged at 14,000 rpm for 20 min and the absorbance at 560 nm of supernatant was measured by a microplate reader. The concentration of unbound rBSA was calculated according to the standard curve of rBSA (C=0.0017Ab+0.0125, in which C is the concentration of rBSA, μg mL⁻¹, Ab is the absorbance at 560 nm). The protein binding efficiency of PAMAM-DEACM with different grafting ratios were calculated according to the following equation:

$$Protein binding efficiency = \frac{{C}_{rBSA} - {C}_{BH - PD / rBSA}}{{C}_{rBSA}} \times 100 \%$$

### 6.5 Synthesis of (5,5-Difluoro-1,3,7,9-tetramethyl-5H-4λ⁴,5λ⁴-dipyrrolo[1,2-c:2′,1′-f][1,3,2]diazabo-rinin-10-yl)methyl Acetate (BODIPY-F₂-OAc) (1)

2-Chloro-2-oxoethyl acetate (0.6 mL, 5.6 mmol, 1.2 equiv) was added to a solution of 2,4-dimethylpyrrole (1.0 mL, 9.3 mmol, 2.0 equiv) in anhydrous dichloromethane (40 mL) under a nitrogen atmosphere. The reaction was stirred under reflux for 3 h. After this time, DIPEA (3.1 mL, 18.6 mmol, 4 equiv) was added. The resulting mixture was allowed to stir at room temperature for another 30 min. Then boron trifluoride diethyl etherate (2.3 mL, 18.6 mmol, 4 equiv) was added and the reaction solution was stirred for 30 min. Then silica was added to the flask, and the solvents were evaporated. BODIPY-F₂-OH was purified by column chromatography by flash chromatography (CombiFlash® system, Teledyne ISCO, Nebraska, USA). The product was obtained as red-gold crystals (814 mg, 45.4% yield). ¹H spectrum is in agreement with published data¹.

### 6.6 Synthesis of (5,5-Difluoro-1,3,7,9-tetramethyl-5H-4λ⁴,5λ⁴-dipyrrolo[1,2-c:2′,1′-f][1,3,2]diazaborinin-10-yl)methanol (BODIPY-F₂-OH)(2)

A mixture of aqueous NaOH solution (0.25 g, 1 mL, 6.25 mmol, 4 equiv) and methanol (29 mL) was dropwise added to a solution of compound 1 (0.5 g, 1.6 mmol) in dichloromethane (15 mL). The reaction mixture was stirred for 2 h in the dark at room temperature. After this time, silica was added to the flask, and the solvents were evaporated. The product was purified by flash chromatography and obtained as a deep red precipitate (230 mg, 51.7% yield). ¹H spectrum is in agreement with published data [60].

### 6.7 Synthesis of (1,3,5,5,7,9-hexamethyl-5H-4λ⁴,5λ⁴-dipyrrolo[1,2-c:2′,1′-f][1,3,2]diazaborinin-10-yl)methanol (BODIPY-Me₂-OH) (3)

Compound 2 (0.31 g, 1.13 mmol) was dissolved in anhydrous diethyl ether (35 mL) under nitrogen atmosphere. Methylmagnesium bromide (5.7 mL, 1 M in tetrahydrofuran. 5.7 mmol, 5 equiv) was dropwise added into the solution. The mixture was stirred in the dark at room temperature for 3 h. Then, the reaction was quenched by dropwise adding water (3 mL). The mixture was extracted with dichloromethane and water three times. The residue was purified by flash chromatography to obtain the product as a red powder. (204 mg, 66.9% yield). ¹H spectrum is in agreement with published data¹. Synthesis of BODIPY-Et₂-OH (4) and BODIPY-Pr₂-OH (5) was similar to the procedure.

### 6.8 Synthesis of (1,3,5,5,7,9-hexamethyl-5H-4λ⁴,5λ⁴-dipyrrolo[1,2-c:2′,1′-f][1,3,2]diazaborinin-10-yl)methyl (4-nitrophenyl) carbonate (BODIPY-Me₂-4NPC) (7)

Compound 3 (120 mg, 0.44 mmol) was dissolved in anhydrous dichloromethane (3 mL) with DIPEA (0.448 mL, 2.22 mmol, 5 equiv) and pyridine (0.165 mL, 1.76 mmol, 4 equiv) at nitrogen atmosphere. Then, a dichloromethane solution of 4-nitrophenyl chloroformate (895 mg, 4.4 mmol, 10 equiv, 3 mL) was dropwise added to the solution of compound 3 at 0° C. in the dark. The reaction mixture was allowed to warm up and was stirred for 4 h. After this time, the solvents were evaporated, and the product was purified by flash chromatography as a light red powder. (140 mg, 73.3% yield). ¹H spectrum is in agreement with published data [60].

### 6.9 Synthesis of BODIPY-Me₂-modified PAMAM (BMMP)

PAMAM was stored in methanol as a 5 wt % solution. Before use, methanol was evaporated to get PAMAM as a transparent gel. Then, anhydrous DMSO (0.5 mL) with DIPEA (2 μL) was added to dissolve PAMAM (10 mg). A solution of BODIPY-Me₂-4NPC dissolved in anhydrous dichloromethane was dropwise added into the solution of PAMAM. The reaction mixture was stirred at room temperature in the dark for 24 h. Then the product was dialyzed (Cutoff MW: 3500 Da) against DMSO until there was no obvious BODIPY fluorescence in the outer fluid. For conjugation of BODIPY with different mole ratios, different amount of BODIPY-Me₂-4NPC was used. Synthesized products were additionally dialyzed against water to remove DMSO and then freeze-dried for ¹H NMR characterization (Bruker DX 500 spectrometer at 400 Hz) and UV-Vis spectrum analysis. Synthesis of BODIPY-F₂-modified-PAMAM (BFMP), BODIPY-Et₂-modified-PAMAM (BEMP), and BODIPY-Pr₂-modified-PAMAM (BPMP) was the same as the above procedure.

### 6.10 Preparation and Characterization of BODIPY-Modified-PAMAM(BMP)/Protein Complexes

The complexes were prepared through flash nanoprecipitation. Briefly, BMP was directly added into protein solutions under vigorous stirring. To further stabilize the complexes, hyaluronic acid and HSA were subsequently added under stirring to protect the complexes from serum. The stable complexes after coating were diluted with water or medium for further characterization. The hydrodynamic diameter and zeta potential of these complexes were characterized by dynamic light scattering (DLS) using Malvern Zetasizer (Nano ZS 90, Malvern, UK). The stability test was conducted in complete DMEM medium with 10% (v/v) fetal bovine serum (FBS) for 48 h at 37° C. The morphology of the nanocomplexes was observed by transmission electron microscope (TEM, Philips, CM100).

### 6.11 Synthesis of Rhodamine B-Labeled Proteins

Briefly, proteins were dissolved in 1 mL phosphate-buffered saline (PBS) buffer as 10 mg/mL solutions, respectively. Then, 100 μL of NaHCO₃solution (1M) was added to the protein stock to adjust the solution pH. Next, 100 μL of rhodamine B isothiocyanate (RBTIC) stock (10 mg/mL, in DMSO) was added, followed by stirring at room temperature for 1 h. After that, the resulting solution was filtered and dialyzed by using a dialysis bag (Cutoff MW: 10000 Da) against PBS buffer under stirring at 4° C. until no obvious fluorescence was detected in the dialysis buffer. Finally, the solution was dialyzed against ddH2O to remove salt and lyophilized to get labeled protein power. The Rhodamine B-labeled proteins were stored at −20° C. before further experiments.

### 6.12 Cell Culture and Cytosolic Protein Delivery

A549 [human lung carcinoma cell line (American Type Culture Collection, ATCC)], and HeLa cells (human cervical carcinoma cell line, ATCC), and Raw264.7 (mouse macrophages, ATCC) were cultured in Dulbecco's Modified Eagle's Medium (DMEM, Gibco). The cell culture media contain 10% fetal bovine serum (FBS, Gibco), and penicillin (100 μg/mL), and streptomycin (100 μg/ml) at 37° C. under 5% CO₂.

Cells were seeded in 24-well plates before cytosolic protein delivery. The protein solutions were mixed with dendrimer, hyaluronic acid (HA), and human serum albumin (HSA) at various weight ratios. After coating, the complex solutions were incubated for 30 min at room temperature to fully stabilize the nanoparticles. Then, the complexes were directly added into the media followed by light irradiation for the light treated group (520 nm Xe Lamp, 10 mW/cm², 5 min). After 4 h incubation, the culture media containing the complexes were removed, and the cells were washed with PBS containing 20 U/mL heparin. The fluorescence intensity of the treated cells was determined by flow cytometry (ACEA NovoCyte Advanteon BVYG). The cellular uptake of differently labeled proteins was also visualized by laser scanning confocal microscopy (Carl Zeiss LSM 900). The time-dependent lysosome escape of rhodamine B labeled protein was observed by LCSM (Carl Zeiss LSM 980). The cells were incubated with HA/HSA coated BMP/rHSA nanoparticles and irradiated once after adding the nanoparticles. After 1 h, 2 h, 4 h, and 16 h incubation, the acid organelles in the treated cells were stained by LysoTracker™ Deep Red (Invitrogen) before confocal imaging. To investigate the light enhanced internalization mechanism of BMP nanoparticles, A549 cells were preincubated with endocytosis inhibitors, methyl-β-cyclodextrin (M-β-CD 10 mM), genistein (400 μM), chlorpromazine (CPZ, 20 μM), and EIPA (20 μM) for 2 h before adding the light-responsive protein nanoparticles.

### 6.13 Cell Culture and Cytosolic Protein Delivery of BSA

To investigate cytosolic delivery of BSA, A549 cells were seeded in 24-well plates in complete DMEM medium for 24 h. Then BH-PD/rBSA nanocomplexes or free rBSA (5 μg) were added. Light-treated groups were immediately irradiated by a 420 nm LED (50 mW/cm², 2 mi). After incubation for 4 h, culture medium was removed, and cells were washed with PBS and collected with trypsin for cellular uptake measurement. The fluorescence intensity of cells was measured by flow cytometry (ACEA NovoCyte Quanteon, CH). Experiments for each group was repeated three times.

To visualize cellular uptake, cells were seeded on 8-well Nunc Lab-Tek chambered slides for 24 h and then cells were treated with BH-PD/rBSA nanocomplexes or free rBSA (2 μg) for different time periods. The light-treated groups were irradiated (420 nm, 50 mW/cm², 2 mi) immediately after addition of the nanocomplexes. Then the cells were washed and fixed with 2.5% paraformaldehyde for 20 min and observed with a confocal microscope (Carl Zeiss LSM 900). Other control groups were treated with protein mixture with PAMAM (equivalent molar ratio to PD0.4) or a commercial Pierce™ Protein Transfection Reagent Kit following the manufacturer's instruction (1 μL kit solution per 2 μg protein). For subcellular localization analysis, cells were seeded onto confocal dishes, additionally treated with 75 nM Lysotracker™ green DND-26 for 90 min, and then observed without fixation.

To investigate HA targeting abilities and endocytosis pathways of the nanocomplexes, A549 cells were pretreated with HA (5 mg/mL) or four endocytosis inhibitors including EIPA (20 μM), genistein (400 μM), chlorpromazine (20 μM) and M-β-CD (10 mM) for 2 h. Then, the cells were incubated with the BH-PD/rBSA nanocomplexes for 2 h before measuring the fluorescence intensity through flow cytometry.

### 6.14 Cytosolic delivery of RNase A, glucose oxidase (GO_{X}), chymotrypsin, and Caspase 3

The cytosolic delivery of RNase A, GO_{X}, and Caspase 3 was tested on Hela cells (RNase A and chymotrypsin) and A549 cells (GO_{X} and Caspase 3). For RNase A and chymotrypsin, the cells were cultured in 96-well plates overnight. RNase A with BMMP and HA/HSA coating were prepared as described above. Then, protein complexes were added to each well. After 1 h incubation in complete media, the plate was irradiated by Xe Lamp (520 nm, 10 mW/cm², 5 min). After overnight incubation, the cytotoxicity of cells was evaluated by MTT assay. For GO_{X}, the cells were also cultured in 96-well plates overnight. GO_{X} with BMMP and HA/HSA coating were prepared as described above. Then, protein complexes were diluted with glucose-free media. The cell culture media were removed and incubated with glucose-free media containing protein complexes and followed with light irradiation. After 4h incubation, the media were replaced with fresh complete media. And the cells were further incubated for 24 h. The cytotoxicity of cells was evaluated by MTT assay. For Caspase 3, the cells were cultured in 12-well plates overnight. Before light irradiation, Caspase 3 BMMP nanoparticles (NPs) with HA/HSA coating were added to each well. After treatment, the cells were washed by PBS containing 20 U/mL heparin. Then, the level of intracellular Caspase 3 and cleaved Caspase 3 was analyzed by western blotting with a commercially available antibody (Anti-Caspase-3 antibody [E87] ab32351, Abcam). Naked RNase A, GO_{X}, Caspase 3, chymotrypsin, and dendrimers at equal concentrations were tested as controls.

### 6.15 Enzymatic activity evaluation of BH-PD/GO_{X}

The enzymatic activity of BH-PD/GO_{X} nanocomplexes was measured following a reported TMB protocol with modification [56]. In general, 10 mU/mL BH-PD/GO_{X} nanocomplexes or GO_{X} were incubated with 0.5 mg/mL glucose and 0.2 mg/mL heparin at 37° C. for 10 min. Then 15 mU/mL HRP and 0.05 mg/mL TMB were added. The absorption at 370 nm was recorded by a microplate reader at 10-s intervals for 10 min. To measure the photo-triggered protein release, nanocomplexes in PBS were irradiated with 420 nm light at 50 mW/cm² for 2 min before measurement. To measure the protein stability, nanocomplexes were preincubated at 37° C. for a certain period before measurement. High concentration of negatively charged heparin was used to compete with PD for protein binding, leading to the dissociation of nanocomplexes and release of proteins [28].

### 6.16 Cytosolic delivery of GO_{X}

Cytosolic delivery of RBITC labeled GO_{X} (rGO_{X}) was measured through flow cytometry and confocal microscopy, as the same methods for evaluation of rBSA delivery. Glucose-free medium was used during the treatment.

### 6.17 Cell viability evaluation of GO_{X} and caspase-3 nanocomplexes

To deliver GO_{X}, BH-PD/GO_{X} nanocomplex or free GO_{X} were diluted with glucose-free media and added to the 96-well plates with A549 cells at different concentrations. Light-treated groups were immediately irradiated with 420 nm light at 50 mW/cm² for 2 min. After 4 h incubation, the cells were washed with PBS to remove extracellular GO_{X} and incubated in complete medium containing glucose for another 20 h. Then cell viability was evaluated by a standard 3-(4,5-dimethylthiazol-2-yl)-2,5-diphenyltetrazolium bromide (MTT) assay with absorbance at 570 nm.

To evaluate toxicity of caspase-3, the PD/caspase-3 nanocomplexes or free caspase-3 were diluted with media without serum and added to the 96-well plates with Hela cells at different concentrations. Light-treated groups were immediately irradiated with 420 nm light at 50 mW/cm² for 2 min. After another 48 h incubation, cell viability was evaluated with MTT assay at 570 nm absorbance. Cell apoptosis and necrosis induced by caspase-3 were also evaluated with an Annexin V-FITC and PI Apoptosis Detection Kit (Biotech) following the manufacturer's protocol and analyzed with flow cytometry (NovoCyte Advanteon BVYG).

### 6.18 Tumor Spheroid Preparation and Tumor Penetration Ability Test

Each spheroid was made by resuspending 8,000 A549 cells in 15 μL media with 0.24% methylcellulose (Dieckmann) by the hanging drop method [61]. After formation, tumor spheroids were collected into 1.5 mL tubes, respectively. Then, free rHSA, PAMAM rHSA, BMMP rHSA NPs, and irradiated BMMP rHSA NPs were directly added into each tube. After 3 h incubation at 37° C., the distribution of rHSA within the 3D spheroids was examined using LSM900 and ZEN software.

### 6.19 Cytosolic Delivery of HRP and Staining

The cytosolic delivery of HRP was investigated by intracellular HRP enzymatic activity assay. Briefly, A549 cells were treated with HA/HSA coated BMP/HRP nanoparticles and irradiated after adding the nanoparticles. After 4 h incubation, the cells were washed five times with PBS containing heparin. Colorless TMB solution turned into a blue product in the presence of HRP and hydrogen peroxide. TMB was dissolved in ethanol and diluted in acetate buffer (pH=5). The subsequent assay was performed as previously reported³. The enzyme activity of HRP was determined by measurement of the absorbance of the blue product at 370 nm by a microplate reader at 30-s intervals for 20 min.

### 6.20 Cytosolic Delivery of Ovalbumin (OVA)

Raw264.7 cells were incubated in a 24-well plate overnight. HA/HSA coated OVA/BMP nanoparticles were added into the media followed by light irradiation (520 nm, 10 mW/cm², 5 min). After 4 h incubation, the cells were washed with PBS and continued to be incubated overnight. Then, the cells were incubated with mouse OVA₂₅₇₋₂₆₄(SIINFEKL) peptide bound to H-2Kb antibody. After that, AF647 anti-mouse rabbit secondary antibody was used for flow cytometry and quantification of presented OVA. For the macrophage/OT-I co-incubation experiment, OVA treated macrophages were incubated with OT-I cells overnight. CellTrace™ CFSE Cell Proliferation Kit was used to detect the activation of OT-I cells according to its provided protocol.

### 6.21 BODIPY-Me2-Modified PAMAM can Deliver Small Molecules

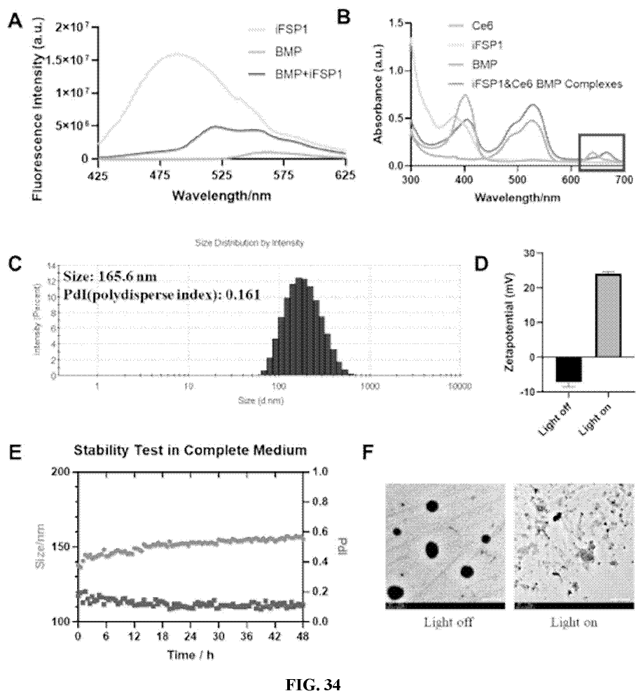

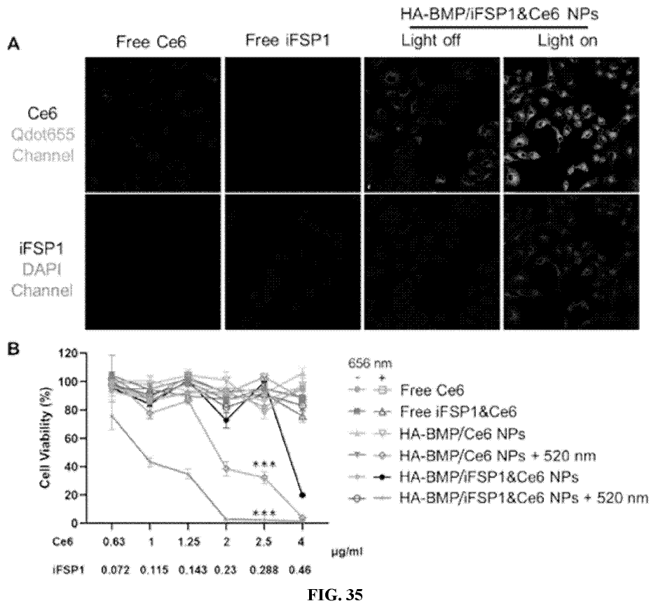

To determine whether the BODIPY-Me₂-modified PAMAM (BMP) could achieve photo-enhanced delivery of hydrophobic small molecules. Two synergistic small molecules, iFSP1 (inhibitor of ferroptosis 1) and Ce6 (Chlorin e6) with fluorescent property were selected as cargos. Fluorescence resonance energy transfer (FRET) is a distance-dependent physical process by which energy is transferred nonradiatively from an excited molecular fluorophore (the donor) to another fluorophore (the acceptor). Due to overlapping of the iFSP1 emission spectrum and the BODIPY excitation spectrum, FRET could be used to determine whether iFSP1 could interact with BMP to form complexes. As shown in FIG. 34A, fluorescence intensity of iFSP1 decreased significantly, indicating strong interaction between BODIPY and iFSP1. Similarly, UV-Vis spectra could reflect interactions between Ce6 and BMP. It was shown in FIG. 34B that the characteristic peak of Ce6 shifted towards long wavelength (in the red box), which indicates Ce6 and BMP could form complexes. After exploring the interactions between BMP and cargo molecules, we successfully prepared BMP/iFSP1&Ce6 nanoparticles (NPs). The NPs were coated with hyaluronic acid (HA) to endow them with negative charge. The NPs showed a size of 165.6 nm with a polydisperse index of 0.161 (FIG. 34C). The NPs were negatively charged while charge reversal happened after light irradiation, implying a potentially strong interaction between negatively charged cell membrane and the positively charged complexes. Without light irradiation, HA-BMP/iFSP1&Ce6 NPs could be stable in complete medium at 37° C. for 48 h (FIG. 34E). Under transmission electron microscope, we observed the NPs showed spherical morphology, while they dissociated into small complexes after light irradiation, which may also promote cellular uptake (FIG. 34F). After preparing and characterizing HA-BMP/iFSP1&Ce6 NPs, we used A549 cells to test whether the NPs could achieve photo-enhanced cellular uptake of cargo drugs and induce cytotoxicity. Through the confocal laser scanning microscopy (CLSM) (FIG. 35A), it was found that fluorescence of both Ce6 and iFSP1 in HA-BMP/iFSP1&Ce6 NPs-treated cells significantly increased after 520-nm light irradiation compared with other groups. Ce6 is a photosensitizer which could respond to 656-nm light to produce singlet oxygen for cancer cell growth inhibition. It was observed in FIG. 35B that the viability of the HA-BMP/iFSP1&Ce6 NPs-treated cells with both 520-nm and 656-nm light irradiation was significantly lower than the ones with other treatments, indicating low cytotoxicity of the carrier, photo-enhanced cellular uptake, and synergism of Ce6 and iFSP1.

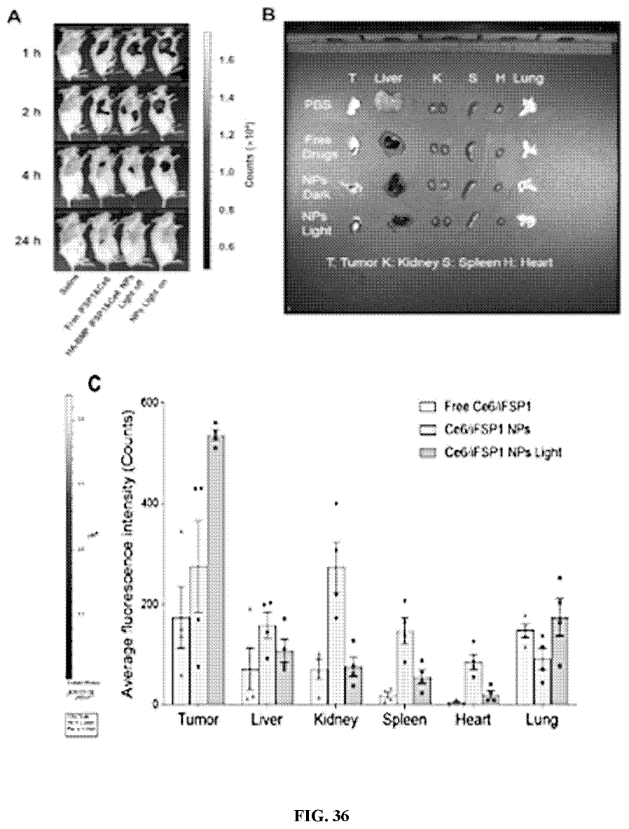

To explore whether this system could be used for in vivo targeting delivery, BALB/c mice were inoculated subcutaneously with 4T1 cells. Free drugs and HA-BMP/iFSP1&Ce6 NPs were injected intravenously. Light irradiation was immediately applied on the tumor sites after NP injection. As shown in FIG. 36A, Ce6 accumulated at the tumor sites detected by IVIS system. The fluorescence intensity in NPs-treated and light-irradiated mice showed stronger fluorescence intensity at the tumor sites, indicating more Ce6 accumulation. At 24 h post-injection and light irradiation, the mice were sacrificed. Major organs and tumor tissues were collected for biodistribution analysis. We found that without HA-BMP/iFSP1&Ce6 NPs treatment or light irradiation, Ce6 accumulated a lot in the liver. However, light irradiation on the tumors after intravenous injection of HA-BMP/iFSP1&Ce6 NPs induced Ce6 accumulation in the tumors (FIGS. 36B and 36C). These results indicated that HA-BMP/iFSP1&Ce6 NPs could achieve targeted delivery of cargo drugs with the help of 520-nm light irradiation in vitro and in vivo.

### 6.22 BODIPY-Me2-Modified PAMAM can Deliver Proteins and Small Molecules Together

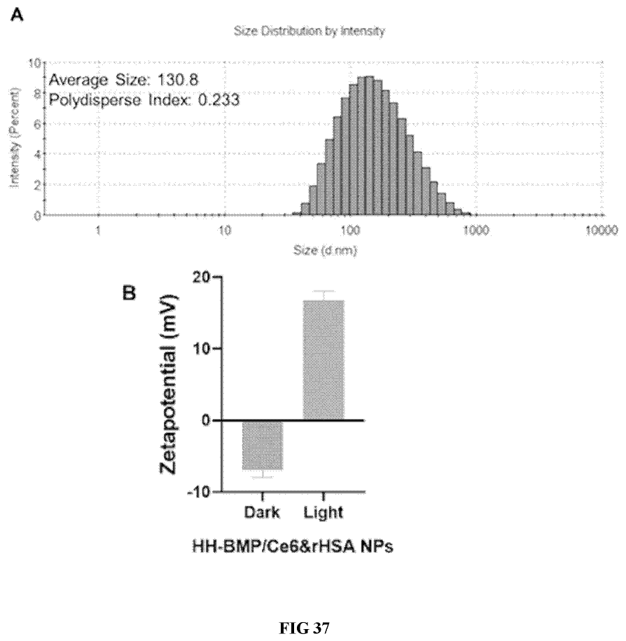

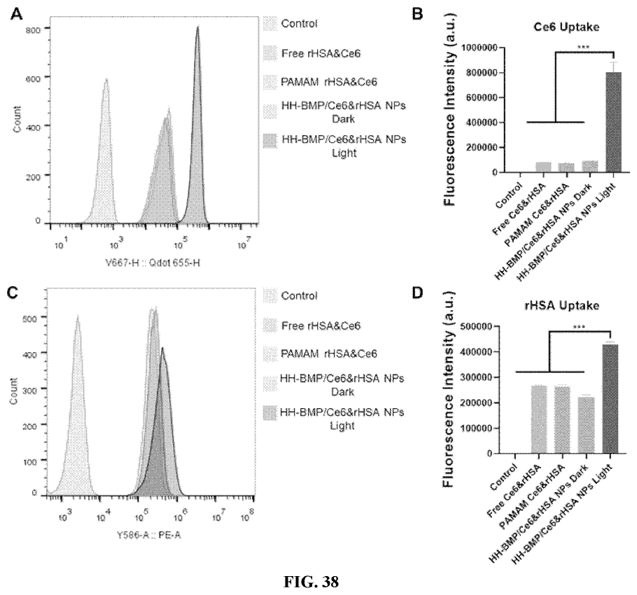

To determine whether the BODIPY-Me₂-modified PAMAM (BMP) could achieve photocontrolled co-delivery of hydrophobic small molecules and hydrophilic proteins, we selected Chlorin e6 (Ce6) and rhodamine B labeled HSA (rHSA) as model cargos for the verification. HA/HSA-coated BMP nanocomplexes encapsulating Ce6 and rHSA (HH-BMP/Ce6&rHSA NPs) could be easily prepared by the nanoprecipitation method. As shown in FIG. 37A, the size of the nanoparticles is 130.8 nm with the polydisperse index (PDI) of 0.233. Importantly, it was found that light irradiation on HH-BMP/Ce6&rHSA NPs induced charge reversal (FIG. 37B) as expected, which could promote the interaction between negatively charged cell membrane and the positively charged particles, thereby increasing cargo uptake. For further demonstration, we used flow cytometry to analyze photoenhanced cargo delivery in 4T1 breast cancer cells. As shown in FIGS. 38A and 38B, light irradiation on HH-BMP/Ce6&rHSA NPs increased the cellular uptake of Ce6 around 10 times. Similarly, the cellular uptake of rHSA was enhanced twice after light irradiation on HH-BMP/Ce6&rHSANPs (FIGS. 38C and 38D). These results indicate that the delivery system could achieve photo-enhanced co-delivery of hydrophobic Ce6 and hydrophilic rHSA. Importantly, the delivery efficiency of the nanocomplexes in the dark was similar to the groups of the mixture of rHSA & Ce6 and the mixture of PAMAM, rHSA & Ce6. This property could reduce the undesired cellular uptake of cargos in the absence of light irradiation, resulting in the reduced side effects of the system. Therefore, it is promising to apply this system for co-delivery of hydrophobic small molecules and hydrophilic proteins for combination therapy.

Exemplary products, systems and methods are set out in the following items:
1. A system for drug delivery comprising a modified poly(amidoamine)(“PAMAM”) comprising a photocleavable compound bound to one or more active agents to form a nanocomplex, wherein the photocleavable compound is 7-diethylamino-4-hydroxymethyl-coumarin (“DEACM”).
2. The system of item 1 further comprising a biodegradable hyaluronic acid (“HA”) and bovine serum albumin (“BSA”) layer.
3. The system of anyone of the preceding items wherein the one or more active agents is an enzyme.
4. The system of anyone of the preceding items wherein the enzyme is glucose oxidase and caspase-3.
5. The system of anyone of the preceding items wherein the one or more active agents is insulin or nimotuzumab.
6. The system of anyone of the preceding items wherein the nanocomplex has a diameter of about 20-200 nm, or about 100-170 nm.
7. A self-assembly system for drug delivery comprising a modified PAMAM conjugated to one or more coumarin derivatives.
8. The system of anyone of the preceding items, wherein the coumarin derivative can be selected from formula (I), formula (II), formula (III), formula (IV), formula (V), or formula (VI):

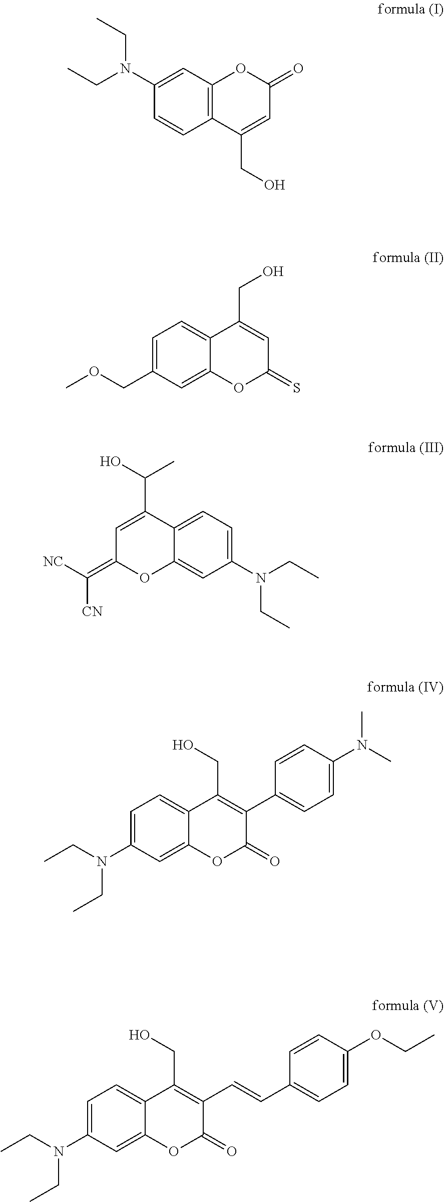

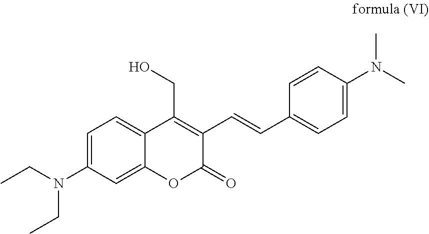 [SMILES: CCN(CC)c1ccc2c(CO)c(/C=C/c3ccc(N(C)C)cc3)c(=O)oc2c1]

9. A method of delivering a drug to a subject at a target site comprising: (i) providing a PAMAM-DEACM compound that is bound to one or more active agents to form a PAMAM-DEACM active agent nanocomplex; (ii) administering the nanocomplex to the subject; and (iii) irradiating the nanocomplex at the target site with a light source to release the one or more active agents.
10. The method of anyone of the preceding items wherein the light source has a wavelength of 350-700 nm.
11. The method of anyone of the preceding items wherein the light source has a wavelength of 420-495 nm.
12. The method of anyone of the preceding items wherein the light source has a wavelength of 620-750 nm.
13. The method of anyone of the preceding items wherein the nanocomplex releases one or more active agents within 2 minutes of light irradiation at 50 mW/cm² or other time periods and irradiances.
14. The method of anyone of the preceding items wherein the target site is intracellular.
15. The method of anyone of the preceding items wherein the target site is the eye or skin.
16. A method of treating cancer in a subject in need thereof comprising: (i) administering a PAMAM-DEACM compound that is bound to glucose oxidase and/or caspase-3 to form a PAMAM-DEACM active agent nanocomplex; and (ii) irradiating the nanocomplex at the target site with a light source to release the glucose oxidase and/or caspase-3.
17. The method of anyone of the preceding items wherein the light source has a wavelength of 420-495 nm.
18. A method for screening coumarin derivatives for delivery of one or more active agents comprising: (i) providing coumarin derivatives conjugating with PAMAM to form PAMAM-coumarins conjugates; (ii) assembling PAMAM-coumarins and the one or more active agents to form a PAMAM-coumarin-active agent nanocomplex; (iii) measuring dispersion of the PAMAM-coumarin-active agent nanocomplex using dynamic light scattering (“DLS”); (iv) quantifying delivery efficiency of the PAMAM-coumarin-active agent nanocomplex; and (v) selecting the PAMAM-coumarin-active agent nanocomplex with the highest delivery efficiency.
19. The method of anyone of the preceding items wherein the coumarin derivatives have a high hydrophobicity and not photocleavable.
20. The method of anyone of the preceding items wherein the PAMAM-coumarin conjugates are PAMAM-coumarin-NEt₂.
21. The method of anyone of the preceding items wherein the PAMAM-coumarin-active agent nanocomplex has more than 60 folds active agent delivery efficiency compared to a free active agent.
22. A self-assembly system for drug delivery comprising a modified PAMAM conjugated to one or more photocleavable compound, wherein the photocleavable compound is BODIPY.
23. The system of anyone of the preceding items, wherein the BODIPY can be selected from formula (VII), formula (VIII), formula (IX), formula (X), formula (XI), or formula (XII):

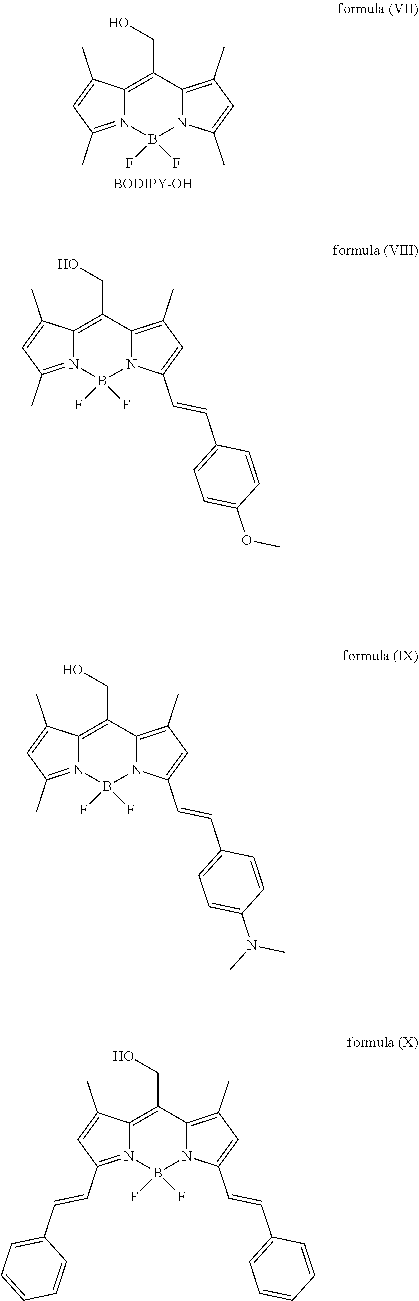

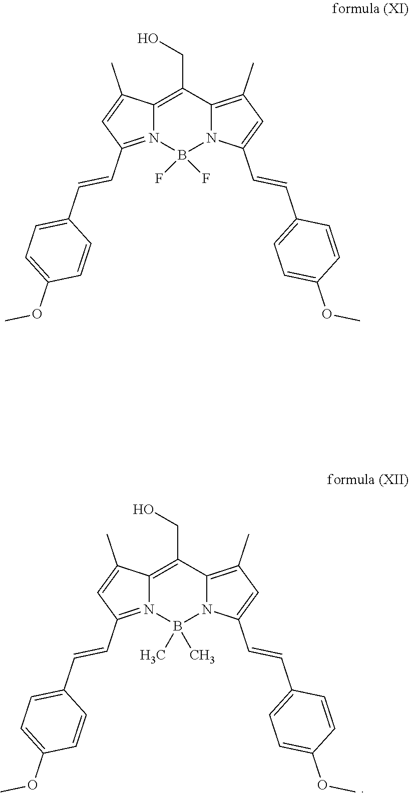

24. A system for drug delivery comprising a BODIPY-modified PAMAM compound (“BMP”) comprising a photocleavable compound BODIPY bound to one or more active agents to form a BODIPY-modified PAMAM active agent nanocomplex.
25. The system of anyone of the preceding items wherein the BMP comprises 2, 14, 26, 50 or 60 BODIPY conjugates forming BMP2, BMP14, BMP26, BMP50 or BMP60, respectively.
26. The system of anyone of the preceding items wherein the BODIPY-modified PAMAM is BODIPY-Me2-modified PAMAM (BMMP), BODIPY-F2-modified PAMAM (BFMP), BODIPY-Et2-modified-PAMAM (BEMP), BODIPY-Pr2-modified-PAMAM (BPMP) or a combination thereof.
27. The system of anyone of the preceding items wherein the BODIPY-modified PAMAM active agent nanocomplex is coated with hyaluronic acid (HA) and human serum albumin (HSA).
28. The system of anyone of the preceding items wherein the BODIPY-modified PAMAM active agent nanocomplex is HHcB60 rHSA.
29. The system of anyone of the preceding items wherein the BODIPY-modified PAMAM active agent nanocomplex has a diameter of 110-150 nm and a polydispersity index (PDI) of 0.090-0.115.
30. The system of anyone of the preceding items wherein the BODIPY-modified PAMAM active agent nanocomplex has a diameter of 122.2 nm and a polydispersity index (PDI) of 0.109.
31. The system of anyone of the preceding items wherein the BODIPY-modified PAMAM active agent nanocomplex has high stability against high ionic strength and binding competition of serum active agents.
32. The system of anyone of the preceding items wherein the BMP comprises 14 BODIPY or 60 BODIPY.
33. The system of anyone of the preceding items wherein the BMP is BMP14 or BMP60.
34. A method of delivering a drug to a subject at a target site comprising: (i) providing a BODIPY-modified PAMAM compound (BMP) that is bound to one or more active agents to form a BODIPY modified PAMAM active agent nanocomplex; (ii) administering the nanocomplex to the subject; and (iii) irradiating the nanocomplex at the target site with a light source to release the one or more active agents.
35. The method of anyone of the preceding items wherein the BMP comprises 2, 14, 26, 50 or 60 BODIPY conjugates forming BMP2, BMP14, BMP26, BMP50 or BMP60 respectively.
36. The method of anyone of the preceding items wherein the BODIPY-modified PAMAM is BODIPY-Me₂-modified PAMAM (BMMP), BODIPY-F₂-modified PAMAM (BFMP), BODIPY-Et₂-modified-PAMAM (BEMP), BODIPY-Pr₂-modified-PAMAM (BPMP) or a combination thereof.
37. The method of anyone of the preceding items wherein the BODIPY-modified PAMAM active agent nanocomplex is coated with hyaluronic acid (HA) and human serum albumin (HSA).
38. The method of anyone of the preceding items wherein the BODIPY-modified PAMAM active agent nanocomplex is HHcB60 rHSA.
39. The method of anyone of the preceding items wherein the BODIPY-modified PAMAM active agent nanocomplex has a diameter of 110-150 nm and a polydispersity index (PDI) of 0.090-0.115.
40. The method of anyone of the preceding items wherein the BODIPY-modified PAMAM active agent nanocomplex has a diameter of 122.2 nm and a polydispersity index (PDI) of 0.109.
41. The method of anyone of the preceding items wherein the BODIPY-modified PAMAM active agent nanocomplex has high stability against high ionic strength and binding competition of serum active agents.
42. The method of anyone of the preceding items wherein the BMP comprises 14 BODIPY or 60 BODIPY.
43. The method of anyone of the preceding items wherein the BMP is BMP14 or BMP60.
44. The method of anyone of the preceding items wherein the light source has a wavelength of 520 nm.
45. The method of anyone of the preceding items wherein the light source has a wavelength of 495-750 nm.
46. The method of anyone of the preceding items wherein the light source has a wavelength of 620-750 nm.
47. The method of anyone of the preceding items wherein the nanocomplex releases one or more active agents within 2 minutes of light irradiation at 50 mW/cm² or other time periods and irradiances.
48. The method of anyone of the preceding items wherein the target site is intracellular.
49. The method of anyone of the preceding items wherein the target site is the eye or skin.
50. A method of treating cancer in a subject in need thereof comprising: (i) administering a BODIPY-modified PAMAM compound that is bound to an active agent to form a BODIPY-modified PAMAM active agent nanocomplex; and (ii) irradiating the nanocomplex at the target site with a light source to release the active agent.
51. The method of anyone of the preceding items wherein the BMP comprises 2, 14, 26, 50 or 60 BODIPY conjugates forming BMP2, BMP14, BMP26, BMP50 or BMP60, respectively.
52. The method of anyone of the preceding items wherein the BODIPY-modified PAMAM is BODIPY-Me₂-modified PAMAM (BMMP), BODIPY-F₂-modified PAMAM (BFMP), BODIPY-Et₂-modified-PAMAM (BEMP), BODIPY-Pr₂-modified-PAMAM (BPMP), or a combination thereof.
53. The method of anyone of the preceding items wherein the BODIPY-modified PAMAM active agent nanocomplex is coated with hyaluronic acid (HA) and human serum albumin (HSA).
54. The method of anyone of the preceding items wherein the active agent is glucose oxidase, caspase-3, horseradish peroxidase, RNase A, or chymotrypsin.
55. The method of anyone of the preceding items wherein the active agent retains its activity.
56. The method of anyone of the preceding items wherein the BODIPY-modified PAMAM active agent nanocomplex is HHcB60 rHSA.
57. The method of anyone of the preceding items wherein the BODIPY-modified PAMAM active agent nanocomplex has a diameter of 110-150 nm and a polydispersity index (PDI) of 0.090-0.115.
58. The method of anyone of the preceding items wherein the BODIPY-modified PAMAM active agent nanocomplex has a diameter of 122.2 nm and a polydispersity index (PDI) of 0.109.
59. The method of anyone of the preceding items wherein the BODIPY-modified PAMAM active agent nanocomplex has high stability against high ionic strength and binding competition of serum active agents.
60. The method of anyone of the preceding items wherein the BMP comprises 14 BODIPY or 60 BODIPY.
61. The method of anyone of the preceding items wherein the BMP is BMP14 or BMP60.
62. The method of anyone of the preceding items wherein the light source has a wavelength of 520 nm.
63. The method of anyone of the preceding items wherein the light source has a wavelength of 495-550 nm.
64. The method of anyone of the preceding items wherein the light source has a wavelength of 620-750 nm.
65. The method of anyone of the preceding items wherein the nanocomplex releases one or more active agents within 2 minutes of light irradiation at 50 mW/cm2 or other time periods and irradiances.
66. The method of anyone of the preceding items wherein the target site is intracellular.
67. The method of anyone of the preceding items wherein the target site is the eye or skin.
68. The method of anyone of the preceding items wherein the target site is a tumor.
69. The method of anyone of the preceding items wherein the target site is an antigen-presenting cell.
70. The method of anyone of the preceding items wherein the antigen-presenting cell is a macrophage.
71. The method of anyone of the preceding items wherein the active agent is OVA₂₅₇₋₂₆₄ peptide (SIINFEKL).

The foregoing description of the specific embodiments will so fully reveal the general nature of the disclosure that others can, by applying knowledge within the skill of the relevant art(s) (including the contents of the documents cited and incorporated by reference herein), readily modify and/or adapt for various applications such specific embodiments, without undue experimentation, without departing from the general concept of the present disclosure. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

While various embodiments of the present disclosure have been described above, it should be understood that they have been presented by way of examples, and not limitation. It would be apparent to one skilled in the relevant art(s) that various changes in form and detail could be made therein without departing from the spirit and scope of the disclosure. Thus, the present disclosure should not be limited by any of the above-described exemplary embodiments but should be defined only in accordance with the following claims and their equivalents.

All references cited herein are incorporated herein by reference in their entirety and for all purposes to the same extent as if each individual publication or patent or patent application was specifically and individually indicated to be incorporated by reference in its entirety for all purposes.

### REFERENCES

[1] M. Ray, Y.-W. Lee, F. Scaletti, R. Yu, V. M. Rotello, Intracellular delivery of proteins by nanocarriers, Nanomedicine 12(8) (2017) 941-952.
[2] X. Liu, F. Wu, Y. Ji, L. Yin, Recent Advances in Anti-cancer Protein/Peptide Delivery, Bioconjug Chem 30(2) (2019) 305-324.
[3] S. Konermann, M. D. Brigham, A. E. Trevino, J. Joung, 0.0. Abudayyeh, C. Barcena, P. D. Hsu, N. Habib, J. S. Gootenberg, H. Nishimasu, O. Nureki, F. Zhang, Genome-scale transcriptional activation by an engineered CRISPR-Cas9 complex, Nature 517(7536) (2015) 583-8.
[4] T. Nochi, Y. Yuki, H. Takahashi, S. Sawada, M. Mejima, T. Kohda, N. Harada, I. G. Kong, A. Sato, N. Kataoka, D. Tokuhara, S. Kurokawa, Y. Takahashi, H. Tsukada, S. Kozaki, K. Akiyoshi, H. Kiyono, Nanogel antigenic protein-delivery system for adjuvant-free intranasal vaccines, Nat Mater 9(7) (2010) 572-8.
[5] R. Goswami, T. Jeon, H. Nagaraj, S. Zhai, V. M. Rotello, Accessing Intracellular Targets through Nanocarrier-Mediated Cytosolic Protein Delivery, Trends Pharmacol. Sci. 41(10) (2020) 743-754.
[6] L. Ren, J. Lv, H. Wang, Y. Cheng, A Coordinative Dendrimer Achieves Excellent Efficiency in Cytosolic Protein and Peptide Delivery, Angew. Chem. Int. Ed. Engl. 59(12) (2020) 4711-4719.
[7] K. Chen, S. Jiang, Y. Hong, Z. Li, Y.-L. Wu, C. Wu, Cationic polymeric nanoformulation: Recent advances in material design for CRISPR/Cas9 gene therapy, Progress in Natural Science: Materials International 29(6) (2019) 617-627.
[8] M. R. Villegas, A. Baeza, M. Vallet-Regi, Nanotechnological Strategies for Protein Delivery, Molecules 23(5) (2018).
[9] Z. Gu, A. Biswas, M. Zhao, Y. Tang, Tailoring nanocarriers for intracellular protein delivery, Chem. Soc. Rev. 40(7) (2011) 3638-55.
[10] F. M. Veronese, PEGylated protein drugs: basic science and clinical applications, Springer 2009.
[11] H. Murata, J. Futami, M. Kitazoe, T. Yonehara, H. Nakanishi, M. Kosaka, H. Tada, M. Sakaguchi, Y. Yagi, M. Seno, N. H. Huh, H. Yamada, Intracellular delivery of glutathione S-transferase-fused proteins into mammalian cells by polyethylenimine-glutathione conjugates, J. Biochem. 144(4) (2008) 447-55.
[12] Y. Jeong, B. Duncan, M. H. Park, C. Kim, V. M. Rotello, Reusable biocatalytic crosslinked microparticles self-assembled from enzyme-nanoparticle complexes, Chem Commun (Camb) 47(44) (2011) 12077-9.
[13] G. H. Gao, M. J. Park, Y. Li, G. H. Im, J. H. Kim, H. N. Kim, J. W. Lee, P. Jeon, O. Y. Bang, J. H. Lee, D. S. Lee, The use of pH-sensitive positively charged polymeric micelles for protein delivery, Biomaterials 33(35) (2012) 9157-64.
[14] P. Yao, Y. Zhang, H. Meng, H. Sun, Z. Zhong, Smart Polymersomes Dually Functionalized with cRGD and Fusogenic GALA Peptides Enable Specific and High-Efficiency Cytosolic Delivery of Apoptotic Proteins, Biomacromolecules 20(1) (2019) 184-191.
[15] B. P. Koppolu, S. G. Smith, S. Ravindranathan, S. Jayanthi, T. K. Suresh Kumar, D. A. Zaharoff, Controlling chitosan-based encapsulation for protein and vaccine delivery, Biomaterials 35(14) (2014) 4382-9.
[16] Y. Lee, T. Ishii, H. Cabral, H. J. Kim, J. H. Seo, N. Nishiyama, H. Oshima, K. Osada, K. Kataoka, Charge-conversional polyionic complex micelles-efficient nanocarriers for protein delivery into cytoplasm, Angew. Chem. Int. Ed. Engl. 48(29) (2009) 5309-12.
[17] M. Vila-Caballer, G. Codolo, F. Munari, A. Malfanti, M. Fassan, M. Rugge, A. Balasso, M. de Bernard, S. Salmaso, A pH-sensitive stearoyl-PEG-poly(methacryloyl sulfadimethoxine)-decorated liposome system for protein delivery: An application for bladder cancer treatment, J Control Release 238 (2016) 31-42.
[18] M. Wang, J. A. Zuris, F. Meng, H. Rees, S. Sun, P. Deng, Y. Han, X. Gao, D. Pouli, Q. Wu, I. Georgakoudi, D. R. Liu, Q. Xu, Efficient delivery of genome-editing proteins using bioreducible lipid nanoparticles, Proc Natl Acad Sci USA 113(11) (2016) 2868-73.
[19] H. Omar, J. G. Croissant, K. Alamoudi, S. Alsaiari, I. Alradwan, M. A. Majrashi, D. H. Anjum, P. Martins, R. Laamarti, J. Eppinger, B. Moosa, A. Almalik, N. M. Khashab, Biodegradable Magnetic Silica@Iron Oxide Nanovectors with Ultra-Large Mesopores for High Protein Loading, Magnetothermal Release, and Delivery, J Control Release 259 (2017) 187-194.
[20] M. Yu, Z. Gu, T. Ottewell, C. Yu, Silica-based nanoparticles for therapeutic protein delivery, Journal of Materials Chemistry B 5(18) (2017) 3241-3252.
[21] X. Qin, C. Yu, J. Wei, L. Li, C. Zhang, Q. Wu, J. Liu, S. Q. Yao, W. Huang, Rational Design of Nanocarriers for Intracellular Protein Delivery, Adv. Mater. 31(46) (2019) e1902791.
[22] J. Lv, Q. Fan, H. Wang, Y. Cheng, Polymers for cytosolic protein delivery, Biomaterials 218 (2019) 119358.
[23] M. Yu, J. Wu, J. Shi, O. C. Farokhzad, Nanotechnology for protein delivery: Overview and perspectives, J Control Release 240 (2016) 24-37.
[24] M. Dabkowska, K. Luczkowska, D. Roginska, A. Sobus, M. Wasilewska, Z. Ulanczyk, B. Machalinski, Novel design of (PEG-ylated)PAMAM-based nanoparticles for sustained delivery of BDNF to neurotoxin-injured differentiated neuroblastoma cells, J Nanobiotechnology 18(1) (2020) 120.
[25] J. Wu, N. Kamaly, J. Shi, L. Zhao, Z. Xiao, G. Hollett, R. John, S. Ray, X. Xu, X. Zhang, P. W. Kantoff, O. C. Farokhzad, Development of multinuclear polymeric nanoparticles as robust protein nanocarriers, Angew. Chem. Int. Ed. Engl. 53(34) (2014) 8975-9.
[26] K. Okuro, M. Sasaki, T. Aida, Boronic Acid-Appended Molecular Glues for ATP-Responsive Activity Modulation of Enzymes, J. Am. Chem. Soc. 138(17) (2016) 5527-30.
[27] H. Chang, J. Lv, X. Gao, X. Wang, H. Wang, H. Chen, X. He, L. Li, Y. Cheng, Rational Design of a Polymer with Robust Efficacy for Intracellular Protein and Peptide Delivery, Nano Lett. 17(3) (2017) 1678-1684.
[28] C. Liu, T. Wan, H. Wang, S. Zhang, Y. Ping, Y. Cheng, A boronic acid-rich dendrimer with robust and unprecedented efficiency for cytosolic protein delivery and CRISPR-Cas9 gene editing, Science advances 5(6) (2019) eaaw8922.
[29] R. Tang, C. S. Kim, D. J. Solfiell, S. Rana, R. Mout, E. M. Velizquez-Delgado, A. Chompoosor, Y. Jeong, B. Yan, Z.-J. Zhu, Direct delivery of functional proteins and enzymes to the cytosol using nanoparticle-stabilized nanocapsules, ACS nano 7(8) (2013) 6667-6673.
[30] R. Mout, M. Ray, G. Yesilbag Tonga, Y. W. Lee, T. Tay, K. Sasaki, V. M. Rotello, Direct Cytosolic Delivery of CRISPR/Cas9-Ribonucleoprotein for Efficient Gene Editing, ACS Nano 11(3) (2017) 2452-2458.
[31] Z. Zhang, W. Shen, J. Ling, Y. Yan, J. Hu, Y. Cheng, The fluorination effect of fluoroamphiphiles in cytosolic protein delivery, Nat Commun 9(1) (2018) 1377.
[32] B. Esteban-Femandez de Avila, D. E. Ramirez-Herrera, S. Campuzano, P. Angsantikul, L. Zhang, J. Wang, Nanomotor-Enabled pH-Responsive Intracellular Delivery of Caspase-3: Toward Rapid Cell Apoptosis, ACS Nano 11(6) (2017) 5367-5374.
[33] H. He, Y. Chen, Y. Li, Z. Song, Y. Zhong, R. Zhu, J. Cheng, L. Yin, Effective and Selective Anti-Cancer Protein Delivery via All-Functions-in-One Nanocarriers Coupled with Visible Light-Responsive, Reversible Protein Engineering, Adv. Funct. Mater. 28(14) (2018).
[34] N. B. Hentzen, R. Mogaki, S. Otake, K. Okuro, T. Aida, Intracellular Photoactivation of Caspase-3 by Molecular Glues for Spatiotemporal Apoptosis Induction, J. Am. Chem. Soc. 142(18) (2020) 8080-8084.
[35] S. Karthik, N. Puvvada, B. N. Kumar, S. Rajput, A. Pathak, M. Mandal, N. D. Singh, Photoresponsive coumarin-tethered multifunctional magnetic nanoparticles for release of anticancer drug, ACS Appl Mater Interfaces 5(11) (2013) 5232-8.
[36] Y. Liang, W. Gao, X. Peng, X. Deng, C. Sun, H. Wu, B. He, Near infrared light responsive hybrid nanoparticles for synergistic therapy, Biomaterials 100 (2016) 76-90.
[37] T. Ohtsuki, S. Kanzaki, S. Nishimura, Y. Kunihiro, M. Sisido, K. Watanabe, Phototriggered protein syntheses by using (7-diethylaminocoumarin-4-yl)methoxycarbonyl-caged aminoacyl tRNAs, Nat Commun 7 (2016) 12501.
[38] Q. Lin, C. Bao, G. Fan, S. Cheng, H. Liu, Z. Liu, L. Zhu, 7-Amino coumarin based fluorescent phototriggers coupled with nano/bio-conjugated bonds: Synthesis, labeling and photorelease, J. Mater. Chem. 22(14) (2012).
[39] W. Wang, Q. Liu, C. Zhan, A. Barhoumi, T. Yang, R. G. Wylie, P. A. Armstrong, D. S. Kohane, Efficient Triplet-Triplet Annihilation-Based Upconversion for Nanoparticle Phototargeting, Nano Lett. 15(10) (2015) 6332-8.
[40] Q. Liu, W. Wang, C. Zhan, T. Yang, D. S. Kohane, Enhanced Precision of Nanoparticle Phototargeting in Vivo at a Safe Irradiance, Nano Lett. 16(7) (2016) 4516-20.
[41] Y. Li, W. Lv, L. Wang, Y. Zhang, L. Yang, T. Wang, L. Zhu, Y. Wang, W. Wang, Photo-triggered nucleus targeting for cancer drug delivery, Nano Research (2021).
[42] C. Bao, L. Zhu, Q. Lin, H. Tian, Building biomedical materials using photochemical bond cleavage, Adv. Mater. 27(10) (2015) 1647-62.
[43] T. Bayraktutan, Y. Onganer, Biophysical influence of coumarin 35 on bovine serum albumin: Spectroscopic study, Spectrochim Acta A Mol Biomol Spectrosc 171 (2017) 90-96.
[44] K. Sharma, P. Yadav, B. Sharma, M. Pandey, S. K. Awasthi, Interaction of coumarin triazole analogs to serum albumins: Spectroscopic analysis and molecular docking studies, J Mol Recognit 33(6) (2020) e2834.
[45] N. Akbay, D. Topkaya, Y. Ergun, S. Alp, E. Gök, Fluorescence study on the interaction of bovine serum albumin with two coumarin derivatives, J. Anal. Chem. 65(4) (2010) 382-387.
[46] S. Paul, R. Ghanti, P. S. Sardar, A. Majhi, Synthesis of a novel coumarin derivative and its binding interaction with serum albumins, Chemistry of Heterocyclic Compounds 55(7) (2019) 607-611.
[47] S. Paul, P. Roy, P. Saha Sardar, A. Majhi, Design, Synthesis, and Biophysical Studies of Novel 1,2,3-Triazole-Based Quinoline and Coumarin Compounds, ACS Omega 4(4) (2019) 7213-7230.
[48] Y. Bibila Mayaya Bisseyou, N. Bouhmaida, B. Guillot, C. Lecomte, N. Lugan, N. Ghermani, C. Jelsch, Experimental and database-transferred electron-density analysis and evaluation of electrostatic forces in coumarin-102 dye, Acta Crystallogr. Sect. B: Struct. Sci. 68(6) (2012) 646-660.
[49] W. Xu, F.-Q. Luo, Q.-S. Tong, J.-X. Li, W.-M. Miao, Y. Zhang, C.-F. Xu, J.-Z. Du, J. Wang, An Intracellular pH-Actuated Polymer for Robust Cytosolic Protein Delivery, CCS Chemistry (2021) 431-442.
[50] F. Barbero, L. Russo, M. Vitali, J. Piella, I. Salvo, M. L. Borrajo, M. Busquets-Fite, R. Grandori, N. G. Bastus, E. Casals, V. Puntes, Formation of the Protein Corona: The Interface between Nanoparticles and the Immune System, Semin. Immunol. 34 (2017) 52-60.
[51] Y. Wang, R. Cai, C. Chen, The Nano-Bio Interactions of Nanomedicines: Understanding the Biochemical Driving Forces and Redox Reactions, Acc. Chem. Res. 52(6) (2019) 1507-1518.
[52] C. D. Spicer, C. Jumeaux, B. Gupta, M. M. Stevens, Peptide and protein nanoparticle conjugates: versatile platforms for biomedical applications, Chem. Soc. Rev. 47(10) (2018) 3574-3620.
[53] W. Zhang, Q. Cheng, S. Guo, D. Lin, P. Huang, J. Liu, T. Wei, L. Deng, Z. Liang, X. J. Liang, A. Dong, Gene transfection efficacy and biocompatibility of polycation/DNA complexes coated with enzyme degradable PEGylated hyaluronic acid, Biomaterials 34(27) (2013) 6495-503.
[54] R. Cai, C. Chen, The Crown and the Scepter: Roles of the Protein Corona in Nanomedicine, Adv. Mater. 31(45) (2019) e1805740.
[55] L. Palanikumar, S. Al-Hosani, M. Kalmouni, V. P. Nguyen, L. Ali, R. Pasricha, F. N. Barrera, M. Magzoub, pH-responsive high stability polymeric nanoparticles for targeted delivery of anticancer therapeutics, Commun Biol 3(1) (2020) 95.
[56] J. Li, Y. Li, Y. Wang, W. Ke, W. Chen, W. Wang, Z. Ge, Polymer Prodrug-Based Nanoreactors Activated by Tumor Acidity for Orchestrated Oxidation/Chemotherapy, Nano Lett. 17(11) (2017) 6983-6990.
[57] W. Tai, P. Zhao, X. Gao, Cytosolic delivery of proteins by cholesterol tagging, Science advances 6(25) (2020) eabb0310.
[58] S. Yang, Q. Tang, L. Chen, J. Chang, T. Jiang, J. Zhao, M. Wang, P. R. Chen, Cationic Lipid-based Intracellular Delivery of Bacterial Effectors for Rewiring Malignant Cell Signaling, Angew. Chem. Int. Ed. Engl. 59(41) (2020) 18087-18094.
[59] M. Wang, S. Sun, C. I. Neufeld, B. Perez-Ramirez, Q. Xu, Reactive oxygen species-responsive protein modification and its intracellular delivery for targeted cancer therapy, Angew. Chem. Int. Ed. Engl. 53(49) (2014) 13444-8.
[60] Leader, B.; Baca, Q. J.; Golan, D. E. Nat Rev Drug Discov 2008, 7, (1), 21-39.
[61] Antosova, Z.; Mackova, M.; Kral, V.; Macek, T. Trends Biotechnol 2009, 27, (11), 628-35.
[62] Kintzing, J. R.; Filsinger Interrante, M. V.; Cochran, J. R. Trends Pharmacol Sci 2016, 37, (12), 993-1008.
[63] Serna, N.; Sanchez-Garcia, L.; Unzueta, U.; Diaz, R.; Vazquez, E.; Mangues, R.; Villaverde, A. Trends Biotechnol 2018, 36, (3), 318-335.
[64] Sharma, G.; Sharma, A. R.; Bhattacharya, M.; Lee, S. S.; Chakraborty, C. Mol Ther 2021, 29, (2), 571-586.
[65] Golchin, A.; Shams, F.; Karami, F. AdvExpMed Biol 2020, 1247, 89-100.
[66] Suzuki, H. Hepatology Research 2002, 22, (1), 1-12.
[67] Krejsa, C.; Rogge, M.; Sadee, W. Nat Rev Drug Discov 2006, 5, (6), 507-21.
[68] Pavlou, A. K.; Reichert, J. M. Nat Biotechnol 2004, 22, (12), 1513-9.
[69] Le Saux, S.; Aubert—Pouëssel, A.; Ouchait, L.; Mohamed, K. E.; Martineau, P.; Guglielmi, L.; Devoisselle, J. M.; Legrand, P.; Chopineau, J.; Morille, M. Advanced Therapeutics 2021, 4, (6).
[70] Wu, D.; Qin, M.; Xu, D.; Wang, L.; Liu, C.; Ren, J.; Zhou, G.; Chen, C.; Yang, F.; Li, Y.; Zhao, Y.; Huang, R.; Pourtaheri, S.; Kang, C.; Kamata, M.; Chen, I. S. Y.; He, Z.; Wen, J.; Chen, W.; Lu, Y. Adv Mater 2019, 31, (18), e1807557.
[71] Ray, M.; Lee, Y. W.; Scaletti, F.; Yu, R.; Rotello, V. M. Nanomedicine (Lond) 2017, 12, (8), 941-952.
[72] D′Astolfo, D. S.; Pagliero, R. J.; Pras, A.; Karthaus, W. R.; Clevers, H.; Prasad, V.; Lebbink, R. J.; Rehmann, H.; Geijsen, N. Cell 2015, 161, (3), 674-690.
[73] Vaishya, R.; Khurana, V.; Patel, S.; Mitra, A. K. Expert Opin Drug Deliv 2015, 12, (3), 415-40.
[74] Fang, Y.; Vadlamudi, M.; Huang, Y.; Guo, X. Pharm Res 2019, 36, (6), 81.
[75] Mastrobattista, E.; Koning, G. A.; van Bloois, L.; Filipe, A. C.; Jiskoot, W.; Storm, G. J Biol Chem 2002, 277, (30), 27135-43.
[76] Aksoy, Y. A.; Yang, B.; Chen, W.; Hung, T.; Kuchel, R. P.; Zammit, N. W.; Grey, S. T.; Goldys, E. M.; Deng, W. ACS Applied Materials & Interfaces 2020, 12, (47), 52433-52444.
[77] Meka, A. K.; Abbaraju, P. L.; Song, H.; Xu, C.; Zhang, J.; Zhang, H.; Yu, M.; Yu, C. Small 2016, 12, (37), 5169-5177.
[78] Tang, R.; Jiang, Z.; Ray, M.; Hou, S.; Rotello, V. M. Nanoscale 2016, 8, (42), 18038-18041.
[79] Yu, C.; Tan, E.; Xu, Y.; Lv, J.; Cheng, Y. Bioconjug Chem 2019, 30, (2), 413-417.
[80] Liu, C.; Wan, T.; Wang, H.; Zhang, S.; Ping, Y.; Cheng, Y. Sci Adv 2019, 5, (6), eaaw8922.
[81] Yan, M.; Du, J.; Gu, Z.; Liang, M.; Hu, Y.; Zhang, W.; Priceman, S.; Wu, L.; Zhou, Z. H.; Liu, Z.; Segura, T.; Tang, Y.; Lu, Y. Nat Nanotechnol 2010, 5, (1), 48-53.
[82] Zhang, S.; Lv, J.; Gao, P.; Feng, Q.; Wang, H.; Cheng, Y. Nano Lett 2021.
[83] Backlund, C. M.; Hango, C. R.; Minter, L. M.; Tew, G. N. ACS Appl Bio Mater 2020, 3, (1), 180-185.
[84] Murugan, B.; Sagadevan, S.; Fatimah, I.; Oh, W.-C.; Motalib Hossain, M. A.; Johan, M. R. Nanotechnology Reviews 2021, 10, (1), 933-953.
[85] Salahpour Anarjan, F. Nano-Structures & Nano-Objects 2019, 19.
[86] Xu, W.; Luo, F.-Q.; Tong, Q.-S.; Li, J.-X.; Miao, W.-M.; Zhang, Y.; Xu, C.-F.; Du, J.-Z.; Wang, J. CCS Chemistry 2021, 431-442.
[87] Lv, J.; Wang, C.; Li, H.; Li, Z.; Fan, Q.; Zhang, Y.; Li, Y.; Wang, H.; Cheng, Y. Nano Lett 2020, 20, (12), 8600-8607.
[88] Zhao, M.; Biswas, A.; Hu, B.; Joo, K. I.; Wang, P.; Gu, Z.; Tang, Y. Biomaterials 2011, 32, (22), 5223-30.
[89] Qiao, Y.; Wan, J.; Zhou, L.; Ma, W.; Yang, Y.; Luo, W.; Yu, Z.; Wang, H. Wiley Interdiscip Rev Nanomed Nanobiotechnol 2019, 11, (1), el 527.
[90] Rwei, A. Y.; Wang, W.; Kohane, D. S. Nano Today 2015, 10, (4), 451-467.
[91] Li, Y.; Zhang, Y.; Wang, W. Nano Research 2018, 11, (10), 5424˜C5438.
[92] Hentzen, N. B.; Mogaki, R.; Otake, S.; Okuro, K.; Aida, T. J Am Chem Soc 2020, 142, (18), 8080-8084.
[93] Pan, Y.; Yang, J.; Luan, X.; Liu, X.; Li, X.; Yang, J.; Huang, T.; Sun, L.; Wang, Y.; Lin, Y.; Song, Y. Sci Adv 2019, 5, (4), eaav7199.
[94] Luo, L.; Guo, Y.; Yang, J.; Liu, Y.; Chu, S.; Kong, F.; Wang, Y.; Zou, Z. Chem Commun (Camb) 2011, 47, (40), 11243-5.
[95] He, H.; Chen, Y.; Li, Y.; Song, Z.; Zhong, Y.; Zhu, R.; Cheng, J.; Yin, L. Advanced Functional Materials 2018, 28, (14).
[96] Gu, Z.; Biswas, A.; Zhao, M.; Tang, Y. Chem Soc Rev 2011, 40, (7), 3638-55.
[97] Li, Y.; Zhou, Y.; Wang, T.; Long, K.; Zhang, Y.; Wang, W. Nanoscale 2021, 13, (42), 17784-17792.
[98] Peterson, J. A.; Wijesooriya, C.; Gehrmann, E. J.; Mahoney, K. M.; Goswami, P. P.; Albright, T. R.; Syed, A.; Dutton, A. S.; Smith, E. A.; Winter, A. H. J Am Chem Soc 2018, 140, (23), 7343-7346.
[99] Slanina, T.; Shrestha, P.; Palao, E.; Kand, D.; Peterson, J. A.; Dutton, A. S.; Rubinstein, N.; Weinstain, R.; Winter, A. H.; Klan, P. J Am Chem Soc 2017, 139, (42), 15168-15175.
[100] Lv, W.; Li, Y.; Li, F.; Lan, X.; Zhang, Y.; Du, L.; Zhao, Q.; Phillips, D. L.; Wang, W. Journal of the American Chemical Society 2019, 141, (44), 17482-17486.
[101] Marfin, Y. S.; Aleksakhina, E. L.; Merkushev, D. A.; Rumyantsev, E. V.; Tomilova, I. K. J Fluoresc 2016, 26, (1), 255-61.
[102] Jameson, L. P.; Smith, N. W.; Annunziata, O.; Dzyuba, S. V. Phys Chem Chem Phys 2016, 18, (21), 14182-5.
[103] Ksenofontov, A. A.; Bocharov, P. S.; Antina, E. V. Journal of Photochemistry and Photobiology A: Chemistry 2019, 368, 254-257.
[104] Lv, J.; He, B.; Yu, J.; Wang, Y.; Wang, C.; Zhang, S.; Wang, H.; Hu, J.; Zhang, Q.; Cheng, Y. Biomaterials 2018, 182, 167-175.
[105] Xu, J.; Lv, J.; Zhuang, Q.; Yang, Z.; Cao, Z.; Xu, L.; Pei, P.; Wang, C.; Wu, H.; Dong, Z.; Chao, Y.; Wang, C.; Yang, K.; Peng, R.; Cheng, Y.; Liu, Z. Nat Nanotechnol 2020, 15, (12), 1043-1052.
[106] Palanikumar, L.; Kim, J.; Oh, J. Y.; Choi, H.; Park, M. H.; Kim, C.; Ryu, J. H. ACS Biomater Sci Eng 2018, 4, (5), 1716-1722.
[107] Iversen, T.-G.; Skotland, T.; Sandvig, K. Nano Today 2011, 6, (2), 176-185.
[108] Rennick, J. J.; Johnston, A. P. R.; Parton, R. G. Nat Nanotechnol 2021, 16, (3), 266-276.
[109] Luo, H. C.; Li, N.; Yan, L.; Mai, K. J.; Sun, K.; Wang, W.; Lao, G. J.; Yang, C.; Zhang, L. M.; Ren, M. Int J Nanomedicine 2017, 12, 1085-1096.
[110] Lonn, P.; Kacsinta, A. D.; Cui, X. S.; Hamil, A. S.; Kaulich, M.; Gogoi, K.; Dowdy, S. F. Sci Rep 2016, 6, 32301.
[111] Freeman, E. C.; Weiland, L. M.; Meng, W. S. J Biomater Sci Polym Ed 2013, 24, (4), 398-416.
[112] Dong, S. X. M.; Caballero, R.; Alt, H.; Roy, D. L. F.; Cassol, E.; Kumar, A. RNA Biol 2020, 17, (6), 755-764.
[113] Olazabal, I. M.; Martin-Cofreces, N. B.; Mittelbrunn, M.; Martinez del Hoyo, G.; Alarcon, B.; Sanchez-Madrid, F. Mol Biol Cell 2008, 19, (2), 701-10.
[114] Xu, J.; Wang, H.; Xu, L.; Chao, Y.; Wang, C.; Han, X.; Dong, Z.; Chang, H.; Peng, R.; Cheng, Y.; Liu, Z. Biomaterials 2019, 207, 1-9.
[115] Sitkowska, K.; Feringa, B. L.; Szymanski, W. J Org Chem 2018, 83, (4), 1819-1827.
[116] Veelken, C.; Bakker, G.-J.; Drell, D.; Friedl, P. Methods 2017, 128, 139-149.
[117] Liu, C.; Wan, T.; Wang, H.; Zhang, S.; Ping, Y.; Cheng, Y. Sci Adv 2019, 5, (6), eaaw8922.

What is claimed is:

1. A system for drug delivery comprising a modified poly(amidoamine)(“PAMAM”) comprising a photocleavable compound bound to one or more active agents to form a nanocomplex, wherein the photocleavable compound is 7-diethylamino-4-hydroxymethyl-coumarin (“DEACM”).

2. The system of claim 1 further comprising a biodegradable hyaluronic acid (“HA”) and bovine serum albumin (“BSA”) layer.

3. The system of anyone of the preceding claims wherein the one or more active agents is an enzyme.

4. The system of anyone of the preceding claims wherein the enzyme is glucose oxidase and caspase-3.

5. The system of anyone of the preceding claims wherein the one or more active agents is insulin or nimotuzumab.

6. The system of anyone of the preceding claims wherein the nanocomplex has a diameter of about 20-200 nm, or about 100-170 nm.

7. A self-assembly system for drug delivery comprising a modified PAMAM conjugated to one or more coumarin derivatives.

8. The system of anyone of the preceding claims, wherein the coumarin derivative can be selected from formula (I), formula (II), formula (III), formula (IV), formula (V), or formula (VI):

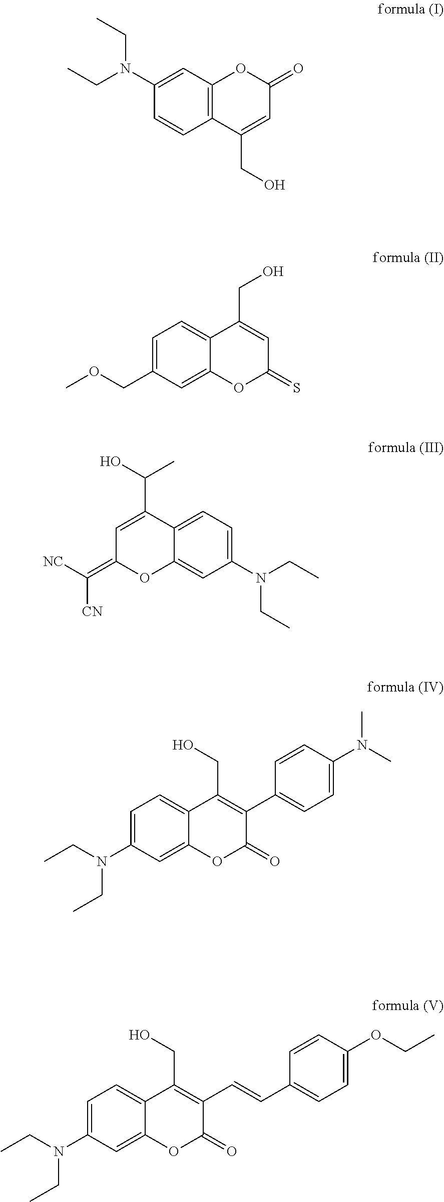

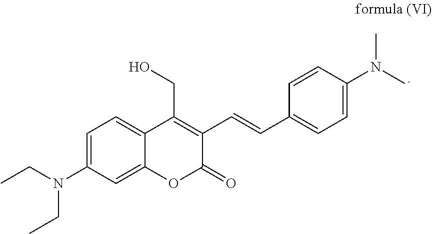 [SMILES: CCN(CC)c1ccc2c(CO)c(/C=C/c3ccc(N(C)C)cc3)c(=O)oc2c1]

9. A method of delivering a drug to a subject at a target site comprising: (i) providing a PAMAM-DEACM compound that is bound to one or more active agents to form a PAMAM-DEACM active agent nanocomplex; (ii) administering the nanocomplex to the subject; and (iii) irradiating the nanocomplex at the target site with a light source to release the one or more active agents.

10. The method of anyone of the preceding claims wherein the light source has a wavelength of 350-700 nm.

11. The method of anyone of the preceding claims wherein the light source has a wavelength of 420-495 nm.

12. The method of anyone of the preceding claims wherein the light source has a wavelength of 620-750 nm.

13. The method of anyone of the preceding claims wherein the nanocomplex releases one or more active agents within 2 minutes of light irradiation at 50 mW/cm² or other time periods and irradiances.

14. The method of anyone of the preceding claims wherein the target site is intracellular.

15. The method of anyone of the preceding claims wherein the target site is the eye or skin.

16. A method of treating cancer in a subject in need thereof comprising: (i) administering a PAMAM-DEACM compound that is bound to glucose oxidase and/or caspase-3 to form a PAMAM-DEACM active agent nanocomplex; and (ii) irradiating the nanocomplex at the target site with a light source to release the glucose oxidase and/or caspase-3.

17. The method of anyone of the preceding claims wherein the light source has a wavelength of 420-495 nm.

18. A method for screening coumarin derivatives for delivery of one or more active agents comprising: (i) providing coumarin derivatives conjugating with PAMAM to form PAMAM-coumarins conjugates; (ii) assembling PAMAM-coumarins and the one or more active agents to form a PAMAM-coumarin-active agent nanocomplex; (iii) measuring dispersion of the PAMAM-coumarin-active agent nanocomplex using dynamic light scattering (“DLS”); (iv) quantifying delivery efficiency of the PAMAM-coumarin-active agent nanocomplex; and (v) selecting the PAMAM-coumarin-active agent nanocomplex with the highest delivery efficiency.

19. The method of anyone of the preceding claims wherein the coumarin derivatives have a high hydrophobicity and not photocleavable.

20. The method of anyone of the preceding claims wherein the PAMAM-coumarin conjugates are PAMAM-coumarin-NEt₂.

21. The method of anyone of the preceding claims wherein the PAMAM-coumarin-active agent nanocomplex has more than 60 folds active agent delivery efficiency compared to a free active agent.

22. A self-assembly system for drug delivery comprising a modified PAMAM conjugated to one or more photocleavable compound, wherein the photocleavable compound is BODIPY.

23. The system of anyone of the preceding claims, wherein the BODIPY can be selected from formula (VII), formula (VIII), formula (IX), formula (X), formula (XI), or formula (XII):

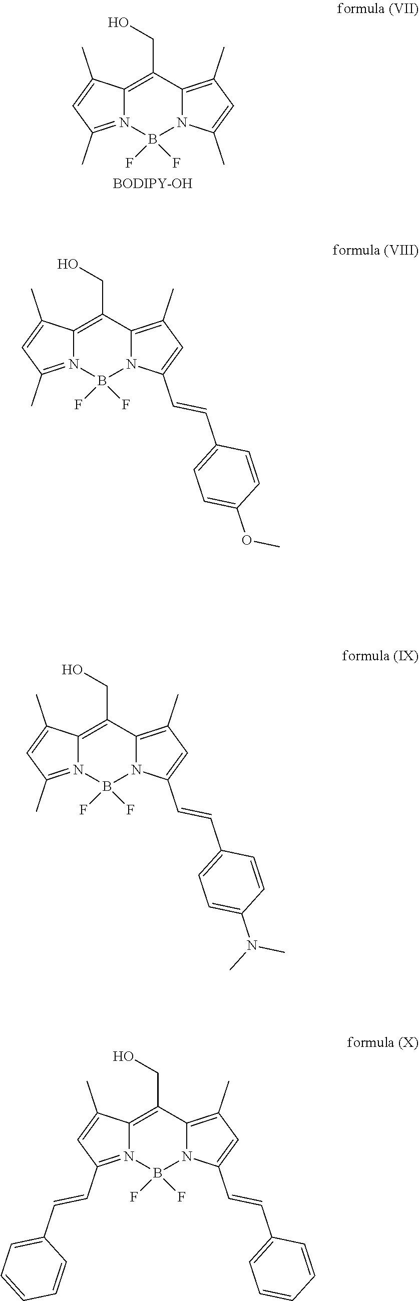

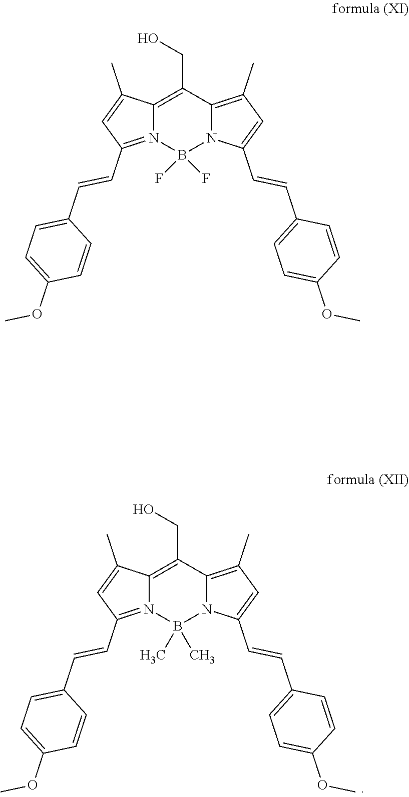

24. A system for drug delivery comprising a BODIPY-modified PAMAM compound (“BMP”) comprising a photocleavable compound BODIPY bound to one or more active agents to form a BODIPY-modified PAMAM active agent nanocomplex.

25. The system of anyone of the preceding claims wherein the BMP comprises 2, 14, 26, 50 or 60 BODIPY conjugates forming BMP2, BMP14, BMP26, BMP50 or BMP60, respectively.

26. The system of anyone of the preceding claims wherein the BODIPY-modified PAMAM is BODIPY-Me2-modified PAMAM (BMMP), BODIPY-F2-modified PAMAM (BFMP), BODIPY-Et2-modified-PAMAM (BEMP), BODIPY-Pr2-modified-PAMAM (BPMP) or a combination thereof.

27. The system of anyone of the preceding claims wherein the BODIPY-modified PAMAM active agent nanocomplex is coated with hyaluronic acid (HA) and human serum albumin (HSA).

28. The system of anyone of the preceding claims wherein the BODIPY-modified PAMAM active agent nanocomplex is HHcB60 rHSA.

29. The system of anyone of the preceding claims wherein the BODIPY-modified PAMAM active agent nanocomplex has a diameter of 110-150 nm and a polydispersity index (PDI) of 0.090-0.115.

30. The system of anyone of the preceding claims wherein the BODIPY-modified PAMAM active agent nanocomplex has a diameter of 122.2 nm and a polydispersity index (PDI) of 0.109.

31. The system of anyone of the preceding claims wherein the BODIPY-modified PAMAM active agent nanocomplex has high stability against high ionic strength and binding competition of serum active agents.

32. The system of anyone of the preceding claims wherein the BMP comprises 14 BODIPY or 60 BODIPY.

33. The system of anyone of the preceding claims wherein the BMP is BMP14 or BMP60.

34. A method of delivering a drug to a subject at a target site comprising: (i) providing a BODIPY-modified PAMAM compound (BMP) that is bound to one or more active agents to form a BODIPY modified PAMAM active agent nanocomplex; (ii) administering the nanocomplex to the subject; and (iii) irradiating the nanocomplex at the target site with a light source to release the one or more active agents.

35. The method of anyone of the preceding claims wherein the BMP comprises 2, 14, 26, 50 or 60 BODIPY conjugates forming BMP2, BMP14, BMP26, BMP50 or BMP60 respectively.

36. The method of anyone of the preceding claims wherein the BODIPY-modified PAMAM is BODIPY-Me₂-modified PAMAM (BMMP), BODIPY-F₂-modified PAMAM (BFMP), BODIPY-Et₂-modified-PAMAM (BEMP), BODIPY-Pr₂-modified-PAMAM (BPMP) or a combination thereof.

37. The method of anyone of the preceding claims wherein the BODIPY-modified PAMAM active agent nanocomplex is coated with hyaluronic acid (HA) and human serum albumin (HSA).

38. The method of anyone of the preceding claims wherein the BODIPY-modified PAMAM active agent nanocomplex is HHcB60 rHSA.

39. The method of anyone of the preceding claims wherein the BODIPY-modified PAMAM active agent nanocomplex has a diameter of 110-150 nm and a polydispersity index (PDI) of 0.090-0.115.

40. The method of anyone of the preceding claims wherein the BODIPY-modified PAMAM active agent nanocomplex has a diameter of 122.2 nm and a polydispersity index (PDI) of 0.109.

41. The method of anyone of the preceding claims wherein the BODIPY-modified PAMAM active agent nanocomplex has high stability against high ionic strength and binding competition of serum active agents.

42. The method of anyone of the preceding claims wherein the BMP comprises 14 BODIPY or 60 BODIPY.

43. The method of anyone of the preceding claims wherein the BMP is BMP14 or BMP60.

44. The method of anyone of the preceding claims wherein the light source has a wavelength of 520 nm.

45. The method of anyone of the preceding claims wherein the light source has a wavelength of 495-750 nm.

46. The method of anyone of the preceding claims wherein the light source has a wavelength of 620-750 nm.

47. The method of anyone of the preceding claims wherein the nanocomplex releases one or more active agents within 2 minutes of light irradiation at 50 mW/cm² or other time periods and irradiances.

48. The method of anyone of the preceding claims wherein the target site is intracellular.

49. The method of anyone of the preceding claims wherein the target site is the eye or skin.

50. A method of treating cancer in a subject in need thereof comprising: (i) administering a BODIPY-modified PAMAM compound that is bound to an active agent to form a BODIPY-modified PAMAM active agent nanocomplex; and (ii) irradiating the nanocomplex at the target site with a light source to release the active agent.

51. The method of anyone of the preceding claims wherein the BMP comprises 2, 14, 26, 50 or 60 BODIPY conjugates forming BMP2, BMP14, BMP26, BMP50 or BMP60, respectively.

52. The method of anyone of the preceding claims wherein the BODIPY-modified PAMAM is BODIPY-Me₂-modified PAMAM (BMMP), BODIPY-F₂-modified PAMAM (BFMP), BODIPY-Et₂-modified-PAMAM (BEMP), BODIPY-Pr₂-modified-PAMAM (BPMP), or a combination thereof.

53. The method of anyone of the preceding claims wherein the BODIPY-modified PAMAM active agent nanocomplex is coated with hyaluronic acid (HA) and human serum albumin (HSA).

54. The method of anyone of the preceding claims wherein the active agent is glucose oxidase, caspase-3, horseradish peroxidase, RNase A, or chymotrypsin.

55. The method of anyone of the preceding claims wherein the active agent retains its activity.

56. The method of anyone of the preceding claims wherein the BODIPY-modified PAMAM active agent nanocomplex is HHcB60 rHSA.

57. The method of anyone of the preceding claims wherein the BODIPY-modified PAMAM active agent nanocomplex has a diameter of 110-150 nm and a polydispersity index (PDI) of 0.090-0.115.

58. The method of anyone of the preceding claims wherein the BODIPY-modified PAMAM active agent nanocomplex has a diameter of 122.2 nm and a polydispersity index (PDI) of 0.109.

59. The method of anyone of the preceding claims wherein the BODIPY-modified PAMAM active agent nanocomplex has high stability against high ionic strength and binding competition of serum active agents.

60. The method of anyone of the preceding claims wherein the BMP comprises 14 BODIPY or 60 BODIPY.

61. The method of anyone of the preceding claims wherein the BMP is BMP14 or BMP60.

62. The method of anyone of the preceding claims wherein the light source has a wavelength of 520 nm.

63. The method of anyone of the preceding claims wherein the light source has a wavelength of 495-550 nm.

64. The method of anyone of the preceding claims wherein the light source has a wavelength of 620-750 nm.

65. The method of anyone of the preceding claims wherein the nanocomplex releases one or more active agents within 2 minutes of light irradiation at 50 mW/cm2 or other time periods and irradiances.

66. The method of anyone of the preceding claims wherein the target site is intracellular.

67. The method of anyone of the preceding claims wherein the target site is the eye or skin.

68. The method of anyone of the preceding claims wherein the target site is a tumor.

69. The method of anyone of the preceding claims wherein the target site is an antigen-presenting cell.

70. The method of anyone of the preceding claims wherein the antigen-presenting cell is a macrophage.

71. The method of anyone of the preceding claims wherein the active agent is OVA₂₅₇₋₂₆₄ peptide (SIINFEKL).
